SEQUENCE LISTING (64 sequences)

SEQ ID NO: 1 - DNA, 21 bp, synthetic construct
  source 1..21: mol_type = other DNA
aagtcatgacaacataggcgg

SEQ ID NO: 2 - DNA, 20 bp, synthetic construct
  source 1..20: mol_type = other DNA
ttcaatcctccttctgcggg

SEQ ID NO: 3 - DNA, 20 bp, synthetic construct
  source 1..20: mol_type = other DNA
tggaccgaggaacaacttgg

SEQ ID NO: 4 - DNA, 20 bp, synthetic construct
  source 1..20: mol_type = other DNA
tcgctcagtccacaaaactg

SEQ ID NO: 5 - DNA, 20 bp, synthetic construct
  source 1..20: mol_type = other DNA
gatttggaatggcatgcggc

SEQ ID NO: 6 - DNA, 20 bp, synthetic construct
  source 1..20: mol_type = other DNA
gcgcatttgatcccgtacaa

SEQ ID NO: 7 - DNA, 20 bp, synthetic construct
  source 1..20: mol_type = other DNA
caacatgcgcgtcattcagg

SEQ ID NO: 8 - DNA, 20 bp, synthetic construct
  source 1..20: mol_type = other DNA
actcgctctggtaatctccg

SEQ ID NO: 9 - DNA, 20 bp, synthetic construct
  source 1..20: mol_type = other DNA
gcgtcatccaggtgtggttt

SEQ ID NO: 10 - DNA, 20 bp, synthetic construct
  source 1..20: mol_type = other DNA
ggtggaccccaacatctcag

SEQ ID NO: 11 - DNA, 19 bp, synthetic construct
  source 1..19: mol_type = other DNA
ggccaagggtcactacacg

SEQ ID NO: 12 - DNA, 21 bp, synthetic construct
  source 1..21: mol_type = other DNA
gcagtcgcagttttcacactc

SEQ ID NO: 13 - DNA, 18 bp, synthetic construct
  source 1..18: mol_type = other DNA
ggccttccgtgttcctac

SEQ ID NO: 14 - DNA, 21 bp, synthetic construct
  source 1..21: mol_type = other DNA
tgtcatcatacttggcaggtt

SEQ ID NO: 15 - DNA, 18 bp, synthetic construct
  source 1..18: mol_type = other DNA
cagcttgcgcaaaagacc

SEQ ID NO: 16 - DNA, 20 bp, synthetic construct
  source 1..20: mol_type = other DNA
taaaaggttgcgcctgaact

SEQ ID NO: 17 - DNA, 19 bp, synthetic construct
  source 1..19: mol_type = other DNA
ccccaacactgaccaacag

SEQ ID NO: 18 - DNA, 18 bp, synthetic construct
  source 1..18: mol_type = other DNA
gtttgctccgccagctta

SEQ ID NO: 19 - DNA, 20 bp, synthetic construct
  source 1..20: mol_type = other DNA
atcgagtgtttccgctgtgt

SEQ ID NO: 20 - DNA, 19 bp, synthetic construct
  source 1..19: mol_type = other DNA
atgggttcggctgccattt

SEQ ID NO: 21 - DNA, 20 bp, synthetic construct
  source 1..20: mol_type = other DNA
cggagcaaagtcttccacct

SEQ ID NO: 22 - DNA, 20 bp, synthetic construct
  source 1..20: mol_type = other DNA
ggacgacaccgagttgagac

SEQ ID NO: 23 - DNA, 16 bp, synthetic construct
  source 1..16: mol_type = other DNA
gccgaaggccgcgatg

SEQ ID NO: 24 - DNA, 20 bp, synthetic construct
  source 1..20: mol_type = other DNA
tgcgtgattttcgccgtttc

SEQ ID NO: 25 - DNA, 20 bp, synthetic construct
  source 1..20: mol_type = other DNA
accctgatggacagccaatg

SEQ ID NO: 26 - DNA, 19 bp, synthetic construct
  source 1..19: mol_type = other DNA
gcatactttgcagccctgg

SEQ ID NO: 27 - DNA, 20 bp, synthetic construct
  source 1..20: mol_type = other DNA
caatcgaaagccagggggaa

SEQ ID NO: 28 - DNA, 20 bp, synthetic construct
  source 1..20: mol_type = other DNA
ctcggctgtcccatactcac

SEQ ID NO: 29 - DNA, 20 bp, synthetic construct
  source 1..20: mol_type = other DNA
gcacaggatgcgtttcagtg

SEQ ID NO: 30 - DNA, 20 bp, synthetic construct
  source 1..20: mol_type = other DNA
tgtctaacccatcgcctcca

SEQ ID NO: 31 - DNA, 21 bp, synthetic construct
  source 1..21: mol_type = other DNA
tgggttcaaagggaaggaagg

SEQ ID NO: 32 - DNA, 20 bp, synthetic construct
  source 1..20: mol_type = other DNA
gaggtgaagtcctgaagccc

SEQ ID NO: 33 - DNA, 15 bp, synthetic construct
  source 1..15: mol_type = other DNA
cggccgcggtcacct

SEQ ID NO: 34 - DNA, 23 bp, synthetic construct
  source 1..23: mol_type = other DNA
cagttcatactgagagcccatct

SEQ ID NO: 35 - DNA, 23 bp, synthetic construct
  source 1..23: mol_type = other DNA
acaggtgacaagaaaaatatcgc

SEQ ID NO: 36 - DNA, 20 bp, synthetic construct
  source 1..20: mol_type = other DNA
cagcgtccagatggttctgt

SEQ ID NO: 37 - DNA, 23 bp, synthetic construct
  source 1..23: mol_type = other DNA
atcgtagagggacggaaaattga

SEQ ID NO: 38 - DNA, 22 bp, synthetic construct
  source 1..22: mol_type = other DNA
gttcccaggcttcttattggtc

SEQ ID NO: 39 - DNA, 20 bp, synthetic construct
  source 1..20: mol_type = other DNA
atggacagtgttcggtctgg

SEQ ID NO: 40 - DNA, 21 bp, synthetic construct
  source 1..21: mol_type = other DNA
agcaccactctgaccaaagat

SEQ ID NO: 41 - DNA, 20 bp, synthetic construct
  source 1..20: mol_type = other DNA
caaaaaccgtgatgccgact

SEQ ID NO: 42 - DNA, 20 bp, synthetic construct
  source 1..20: mol_type = other DNA
cgccctcaggttttctctgt

SEQ ID NO: 43 - DNA, 20 bp, synthetic construct
  source 1..20: mol_type = other DNA
aaacccaagcaggtgacaac

SEQ ID NO: 44 - DNA, 20 bp, synthetic construct
  source 1..20: mol_type = other DNA
gcgtccttgagcaatttgtc

SEQ ID NO: 45 - DNA, 20 bp, synthetic construct
  source 1..20: mol_type = other DNA
tggccaaactgctgttgatt

SEQ ID NO: 46 - DNA, 20 bp, synthetic construct
  source 1..20: mol_type = other DNA
taggcttgtcccgtttccac

SEQ ID NO: 47 - DNA, 20 bp, synthetic construct
  source 1..20: mol_type = other DNA
acgttagggctgttccaagg

SEQ ID NO: 48 - DNA, 20 bp, synthetic construct
  source 1..20: mol_type = other DNA
ggcaagggggtaggatgaac

SEQ ID NO: 49 - DNA, 20 bp, synthetic construct
  source 1..20: mol_type = other DNA
actacgttgctactggcact

SEQ ID NO: 50 - DNA, 20 bp, synthetic construct
  source 1..20: mol_type = other DNA
gttcacgatgccccaccatc

SEQ ID NO: 51 - DNA, 20 bp, synthetic construct
  source 1..20: mol_type = other DNA
tgatgtagacgcaagggggt

SEQ ID NO: 52 - DNA, 23 bp, synthetic construct
  source 1..23: mol_type = other DNA
catgtaccaccatgaagagctga

SEQ ID NO: 53 - DNA, 20 bp, synthetic construct
  source 1..20: mol_type = other DNA
ctacccgatctggtggtcct

SEQ ID NO: 54 - DNA, 20 bp, synthetic construct
  source 1..20: mol_type = other DNA
acagagaatgggctgagtgc

SEQ ID NO: 55 - DNA, 22 bp, synthetic construct
  source 1..22: mol_type = other DNA
caggaaggccatcttgacacac

SEQ ID NO: 56 - DNA, 20 bp, synthetic construct
  source 1..20: mol_type = other DNA
gtctttacctcgcaggagcc

SEQ ID NO: 57 - DNA, 20 bp, synthetic construct
  source 1..20: mol_type = other DNA
ccggccacctttctctctta

SEQ ID NO: 58 - DNA, 18 bp, synthetic construct
  source 1..18: mol_type = other DNA
cggagctcccacgctgat

SEQ ID NO: 59 - DNA, 20 bp, synthetic construct
  source 1..20: mol_type = other DNA
acggtttaagccccaagagc

SEQ ID NO: 60 - DNA, 18 bp, synthetic construct
  source 1..18: mol_type = other DNA
gagtcagtcagcgcctcc

SEQ ID NO: 61 - DNA, 20 bp, synthetic construct
  source 1..20: mol_type = other DNA
gctgcgctttgataaggtcc

SEQ ID NO: 62 - DNA, 20 bp, synthetic construct
  source 1..20: mol_type = other DNA
cgcgaacggctgcttatttt

SEQ ID NO: 63 - DNA, 18 bp, synthetic construct
  source 1..18: mol_type = other DNA
ggacgggagcacacttcg

SEQ ID NO: 64 - DNA, 18 bp, synthetic construct
  source 1..18: mol_type = other DNA
gccttcaggccgtcctct

(19) United States
(12) Patent Application Publication
(10) Pub. No.: US 2026/0275280 A1
(43) Pub. Date: Sep. 17, 2026

(54) DIRECTED DIFFERENTIATION PROTOCOLS TO DERIVE BONE FIDE SPINAL SENSORY INTERNEURONS

(71) Applicant: THE REGENTS OF THE UNIVERSITY OF CALIFORNIA, OAKLAND, CA (US)

(72) Inventors: Samantha J. Butler, LOS ANGELES, CA (US); Sandeep Gupta, LOS ANGELES, CA (US)

(73) Assignee: THE REGENTS OF THE UNIVERSITY OF CALIFORNIA, OAKLAND, CA (US)

(21) Appl. No.: 18/868,444

(22) PCT Filed: May 25, 2023
(86) PCT No.: PCT/US23/67492

§ 371 Date: Nov. 22, 2024

Related U.S. Application Data
(60) Provisional application No. 63/365,302, filed on May 25, 2022.

(51) Int. Cl. C12N 5/0793; A61K 35/30

(52) U.S. Cl. CPC C12N 5/0619; A61K 35/30; C12N 2500/38; C12N 2501/115; C12N 2501/155; C12N 2501/415; C12N 2503/02; C12N 2506/02; C12N 2513/00

(57) ABSTRACT

Methods for deriving dorsal interneurons (dIs) of all types, dI1-dI6, and for producing dIs in large quantities without changing their identity, are described. A method for producing dIs in vitro comprises contacting neuromesodermal progenitor cells (NMPs) with specific factors following two variations on a protocol. The methods described herein provide a disease model for sensory processing disorders, such as fibromyalgia, and autism. Cells can be obtained from an individual subject using a skin biopsy to develop the stem cells that can then be used for study and testing that is tailored to the individual subject.

This application claims benefit of U.S. provisional patent application No. 63/365,302, filed May 25, 2022, the entire contents of which are incorporated by reference into this application.

### ACKNOWLEDGEMENT OF GOVERNMENT SUPPORT

This invention was made with government support under NS085097, awarded by the National Institutes of Health. The government has certain rights in the invention.

### REFERENCE TO A SEQUENCE LISTING

The content of the XML file of the sequence listing named “UCLA287_seq”, which is 57 kb in size, created on May 25, 2023, and electronically submitted herewith the application, is incorporated herein by reference in its entirety.

### BACKGROUND

Spinal cord injuries (SCIs) can result in the loss of both coordinated movement and somatosensation (lonta et al., 2016; Lenggenhager et al., 2012), and the ability to perceive the environment. Without somatosensation, patients can no longer communicate through touch, sense danger through pain, or control the functions of the gut and bladder (Furusawa et al., 2011). While stem cell-based cellular replacement therapies have shown promise for SCI (Antonic et al., 2013; Kong et al., 2021), the focus so far has been on the recovery of coordinated motor function (Faravelli et al., 2014; Trawczynski et al., 2019). Less attention has been paid to regaining sensory functions, despite their critical roles ensuring physical and emotional well-being, and providing modulatory feedback to the motor system through proprioceptive pathways. Somatosensory information is received peripherally and then relayed centrally by different populations of dorsal interneurons (dIs) in the spinal cord. Each dI population is specialized for distinct somatosensory modalities, and forms microcircuits that span different layers of the dorsal horn (Gupta and Butler, 2021; Lai et al., 2016). For example, dI1s (proprioceptors) (Lai et al., 2011), dI2s and dI3s (both mechanosensors) (Bui et al., 2013; Haimson et al., 2020) and dI6 (gait) (Perry et al., 2019) are located in the deep layers of the dorsal horn, while dI4s and dI5s regulate pain, heat, and itch in the superficial layers of the dorsal horn (Koch et al., 2018). To replace damaged spinal tissue, it will be critical to derive a complete complement of dorsal sensory interneurons that molecularly and functionally phenocopy endogenous dIs.

There thus remains a need for methods of obtaining dI of each type, dI1-dI6, and in sufficient quantities for therapeutic recovery of somatosensory functions.

### SUMMARY

The methods described herein provide novel methods for deriving dorsal interneurons (dIs) of all types, dI1-dI6, as well as methods for producing dIs in large quantities without changing their identity, and methods for using same. For example, described herein is a method for producing dorsal spinal interneurons (dI) in vitro. In some embodiments, the method comprises contacting a population of neuromesodermal progenitor cells (NMPs) with retinoic acid (RA) for a sufficient time to neuralize the NMPs into dorsal neural progenitor cells (NPCs); and culturing the dorsal NPCs with retinoic acid (RA) for an additional day, and optionally, further contacting the dorsal NPCs with bone morphogenic protein 4 (BMP4) for the additional day. The contacting with RA is performed in the absence of BMP4. Typically, a sufficient time to neuralize the NMPs is about one day of contact with RA in the absence of BMP4. NMPs can be derived from embryonic stem cells (ESCs) by contacting the ESCs with basic fibroblast growth factor (bFGF) and an activator of the Wnt/beta-catenin pathway, such as CHIR, typically for about 3 days. In some embodiments, the ESCs are contacted with bGFG for two days, followed by one day of contact with both bFGF and CHIR. The timing for neuralization and differentiation can be adjusted as described herein for use with human cells.

In some embodiments, the method of producing dIs comprises contacting a population of dorsal neural progenitor cells (NPCs) with an activator of the Wnt/beta-catenin pathway; and culturing the dorsal NPCs of (a) under conditions permitting proliferation, wherein the dorsal NPCs maintain their dI fate potential. In some embodiments, the activator of the Wnt/beta-catenin pathway is CHIR (C₂₂H₁₈Cl₂N₈; CAS No. 252917-06-9). In some embodiments, the dorsal NPCs are obtained by contacting a population of NMPs with retinoic acid (RA) for about two to about 20 days. In some embodiments, the contacting with RA continues for up to ten days. The contact with the activator of the Wnt/beta-catenin pathway is made with cells that are NPCs, rather than ESCs, to maintain the dI fate potential. Activation of the Wnt signaling with ESCs diverts stem cells towards the mesodermal fate when the ESCs have not yet been treated with retinoic acid (RA). In some embodiments, the contacting with RA is for about two days. In some embodiments, the contacting with RA is sufficient to direct the cells to a NPC state by day 4. In some embodiments, the contacting further comprises contacting with bone morphogenic protein 4 (BMP4) for the second of the two days. In some embodiments, the contacting with BMP4 continues for up to 10 days (e.g., the latter portion of the contacting with RA for, e.g., 20 days). In some embodiments, the contacting with BMP4 is for about 24 hours.

In some embodiments, the method further comprises subsequently ceasing the contacting with an activator of the Wnt/beta-catenin pathway, followed by contacting the dorsal NPCs with retinoic acid (RA) for about two days, optionally for up to 20 days. In some embodiments, the contacting with an activator of the Wnt/beta-catenin pathway occurs for up to 10 days. In some embodiments, the NPCs are further contacted with bone morphogenic protein 4 (BMP4) for the second of the two days, and, optionally, for up to 10 days (e.g., the second half of a 20 day protocol). In some embodiments, the dorsal NPCs comprise a plurality of each of dI1, dI2, dI3, dI4, dI5, and dI6 phenotypes. In some embodiments, the dorsal NPCs contacted with RA express Pax3 and Pax7, and the dorsal NPCs contacted with RA and BMP4 express Pax3 and Olig3. In some embodiments, the dorsal NPCs express one or more dI markers listed in Table 4. In some embodiments, the activator of the Wnt/beta-catenin pathway (e.g., CHIR) is withdrawn. Ceasing the contact with the activator of the Wnt/beta-catenin pathway induces differentiation with mixed dI identities. Treatment of the NPCs post-withdrawal with RA and with RA+BMP4 is used to direct the differentiation to the desired dI types.

In some embodiments, the population of dIs produced by the method described herein comprises at least 5% dI1, dI2, dI3, dI4, dI5, and/or dI6, wherein at least 5% of the cells in the population are of the indicated type of dI. In some embodiments, the population of dIs produced by the method described herein comprises at least 10% of dI1, dI2, dI3, dI4, dI5, and/or dI6. In some embodiments, the population of dIs produced by the method described herein comprises at least 5% of each of dI1, dI2, dI3, dI4, dI5, and dI6. In some embodiments, the population of dIs produced by the method described herein is enriched for one or more types of dIs 1-6. In some embodiments, the method described herein produces dI2 and/or dI5 interneurons.

In some embodiments, the culturing NPCs is three-dimensional culturing. Three-dimensional culturing is used to obtain embryoid bodies (EBs), which have been shown to form functional sensory circuits. These EBs provide an ex vivo model for disorders affecting sensation. In some embodiments, the NMPs are obtained from a population of induced pluripotent stem cells (iPSCs). The iPSCs can be obtained from a subject used to direct patient-specific EBs containing either healthy or mutant dIs. These new classes of protocols also provide an unlimited source of bonafide human dIs for cellular replacement therapies to reestablish sensory connections in injured patients.

Also described herein is a method of screening neuroactive agents. In some embodiments, the method comprises contacting a population of dorsal spinal interneurons (dIs) produced by the above method with a candidate neuroactive agent; and measuring a change in physicochemical properties of the dis relative to a reference population of dIs, wherein a neuroactive agent is identified when a change relative to the reference population of dIs is measured. In some embodiments, the candidate neuroactive agent is an analgesic, and the population of dIs is enriched for dI4 and/or dI5. In some embodiments, the population of dIs has been enriched for dI4 and dI5 by contacting the dorsal NPCs with retinoic acid (RA) for about two days, and subsequently culturing the dorsal NPCs in the absence of growth factor. In some embodiments, the candidate neuroactive agent is a psychoactive compound. In some embodiments, the neuroactive agent is cocaine or amphetamine. In some embodiments, the dI are murine or human dI. In some embodiments, the screening is performed in vitro or ex vivo.

The types of dIs and their associated functions are as follows: dI1—proprioception; dI2—proprioception/gait stabilization; dI3—touch/motor control (grip strength); dI4—pain/itch; dI5—touch/pain/itch; and dI6—motor control. Thus each population of dIs is tuned to a different sensory modality (or modalities) and each one represents a different potential drug screening target depending on the objective (i.e. looking to suppress pain, itch, or to alter balance). One can screen for a variety of agents using any one or a combination of dIs, selected based on the desired objective, e.g., in screening for compounds that specifically activate or suppress neural activity in the different dIs.

Also provided is a method of transplanting dorsal NPCs to the spinal cord of a subject in need thereof. In some embodiments, the method comprises administering dorsal NPCs produced by one of the methods described herein to the subject. In some embodiments, the dorsal NPCs are administered by injection to the spinal cord of the subject. The dIs or dorsal NPCs produced by the methods described herein provide a means of cellular therapy to replace diseased or damaged spinal tissues, and thereby restore sensory modalities.

Restoration of some or all of the missing sensory modalities can be achieved by selecting particular dIs (or dPs) to be transplanted.

### DESCRIPTION OF THE DRAWINGS

FIG. 1. Distinct temporal combinations of RA and BMP4 direct different mESC identities. (A) Schematic patterning signals in the early spinal cord. Dorsal neural progenitor cells (NPCs) arise in response to two growth factors, retinoic acid (RA, lateral) and the bone morphogenetic protein (BMPs, upper portion) family, which are secreted from adjacent tissue, i.e. the somites and roof plate respectively. Shh refers to sonic hedgehog (lower portion). (B) A previously described protocol for generating ventral spinal progenitors through a neuromesodermal (NMP) intermediate state (Gouti et al., 2014) was modified to direct mESCs towards dorsal spinal cord identities. Three dorsalizing conditions were assessed: in protocol 1, NMPs were solely treated with RA for 48 hours, starting from day 3. In protocol 2, NMPs were treated with both RA and BMP4, for day 3 and 4. Finally, in protocol 3, NMPs were exposed to 48 hours of RA from day 3, with a 24-hour pulse of BMP4 at day 4. (C-E) By day 5, immunohistochemical analyses show that antibodies directed against phosphorylated (p) Smad1/5/8 (pSmad1/5/8) decorate nuclei in cells grown under protocol 2 and 3, but not in protocol 1, confirming that the BMP signaling pathway is active. (F-K) By day 9, cells in protocol 1 and 3, but not protocol 2, form rosette-like structures (arrowheads, F, H). The rosettes express pan-neural markers, including Sox2 (I, K), which is present in NPCs, and Tuj1 (β-tubulin III, arrowheads, I, K) which is present in all neurites. The rosettes also co-express Pax3 (F, H) and Pax6, suggesting the presence of dorsal NPCs. In contrast, cells grown under protocol 2 do not express NPC markers (G, J). (L) Timeline for bulk-RNA seq sample acquisition (n=3). (M) A principal component (PC) analysis of all timepoints revealed that protocol 1 (R-branch) and 3 (B-branch) direct mESCs-derived NMPs to follow similar trajectories. In contrast, protocol 2 directs NMPs towards the distinct transcriptional trajectory (C-branch). (N) A Pearson correlation analysis of the transcriptome of differentiated cells at day 9, confirmed that protocol 1 (R9) and 3 (B9) produce transcriptionally similar cells, which are distinct from the cells generated in protocol 2 (C9). (O) A weighted gene co-expression analysis identified that Eigengene modules with decreasing expression, i.e., highest on day 0 and lowest on day 9, belong to GO categories related to rRNA biogenesis, which is implicated in the loss of pluripotency (Watanabe-Susaki et al., 2014; Woolnough et al., 2016). Conversely, neural-specific Eigengene modules are sequentially upregulated in the R and B branches, respectively, while a cardiac-specific gene module is sequentially upregulated in the C-branch. See also FIG. 8(E), for details of the other gene modules. Scale bar: C-K, 100 μm.

FIG. 2. Single cell sequencing identifies relevant spinal function-specific modules in in vitro-derived-dIs. (A) Timeline of protocol 1 and 3. On day 9 of the differentiation procedure, cells were processed for single-cell (sc) RNA sequencing (n=1). (B) Schematic transverse section of the spinal cord, showing the position of the dorsal progenitor (dP) domains, and post-mitotic dorsal interneurons (dIs). The six classes of dIs mediate distinct functionalities and can be identified by specific combinations of transcription factors. (C, D) Uniform manifold approximation and projection (UMAP) plots of single cells sequenced at day 9 under RA (C) and RA+BMP4 conditions (D). Feature plots show the distribution of Sox2+NPCs and Tubb3 (Tuj1)+differentiated neurons in the cell clusters. (E) Reclustering of the Tubb3+ neuronal clusters shown in C, using the unsupervised clustering algorithm on Seurat, identified 14 clusters (3394 cells). Feature plots show the distribution of the Pax2+Lhx1+dI4/dI6, Lmx1b+dI5 and Dmrt3+dI6 populations. Based on this dI-specific gene expression analysis, 5.2% of the Tuj1+ neurons are dI1, 5% are dI2, 33% are dI4, 40.6% are of dI5s and 7.6% are dI6s. ~8.5% of neuronal cells consist of neurofilament (NF) expressing cells that do not express any dI specific markers (see Table 1). Concurring with the in vivo functionality (B), the dI4/dI6 clusters also express Gad2, a marker of inhibitory neurons, while the dI5 clusters express SIc17a6 (vGlut2), present in excitatory neurons. (F) Sub-clustering of the Tuj1+ neuronal clusters shown in D, identified 13 clusters (3967 cells). Feature plots show gene expression specific to Lhx2+BarhI1+dI1, Foxd3+dI2 and IsI1+dI3 populations. Based on the dI-specific gene expression analysis, 45.5% of the Tuj1+ neurons are dI1, ~29% are dI2, ~8.1% are dI3, ~7.8% are dI4. 9.5% of the Tuj1+cells are of unknown neural identity (see Table 1, cluster 3). Again, concurring with the known in vivo functionality (B), the dI1, dI2 and dI3 clusters all express SIc17a2 and Grin2b, markers of excitatory neurons. (G) Gene ontology (GO) analysis of the genes upregulated (Log fold change >0.2) in the indicated dI clusters identified in E and F. Gene lists were subjected to the DAVID functional annotation tool to identify the significantly enriched biological processes and pathway terms. These analyses reveal that relevant sensory/motor functions are enriched in the clusters of in vitro derived dIs.

FIG. 3. Stem cell-derived sensory interneurons resemble their endogenous counterparts. (A) UMAP plot of the in vitro differentiation sc-Seq dataset integrated with an in vivo dataset of embryonic spinal cord (E9.5-E13.5) (Delile et al., 2019) using reciprocal PCA methodology in Seurat V4. The combined dataset was then processed and embedded into a three-dimensional UMAP space. Only UMAP1 and UMAP3 are shown here in the two-dimensional plots. Cell type labels in the in vivo spinal cord dataset were then simplified and projected onto the in vitro dataset. (B) UMAP plot colored by dataset, highlighting overlap of the in vitro cells with the in vivo cells. (C) Clustering of the integrated dataset yields 24 shared clusters, with 6 clusters being unique to the in vivo spinal cord dataset. (D) Dot plot showing the expression levels in the integrated dataset of the genes that mark different dI types in the spinal cord. Only clusters shared between the in vivo and in vitro datasets are shown. (E) Integration of the in vivo spinal cord and in vitro datasets with reference datasets (lung, kidney, and trachea) from the Tabula Muris Consortium show the in vitro data overlapping only with spinal cord in vivo dataset. Datasets chosen from Tabula Muis were ones isolated by microfluidic means (as opposed to FACS) to minimize technical factors in the analysis. (F) UMAP plots of the neuronal portion of the integrated datasets. The in vivo spinal cord dataset was colored by the published annotations (Delile et al., 2019) to reveal the locations of the different dI populations. Each dI population was then overlaid onto the in vitro datasets (black) from either protocol 1 (RA) or 3 (RA+BMP4). The most substantial overlap was seen for RA+BMP4 with dI1 and dI2; and RA with dI4, dI5 and dI6. No overlap was observed for motor neurons (pink) with the two in vitro datasets. (G) dI-specific cells were extracted from both the in vitro protocols and in vivo spinal cord dataset, and then plotted in the integrated UMAP space with in vitro cells on top of in vivo cells.

FIG. 4. Wnts are upregulated as an immediate response to BMP4 signaling in mESCs-derived NPCs. (A) Timelines for sample collection for bulk RNA-Seq analysis of protocol 3 (B) to investigate the transcriptomic changes induced by 6 hrs (day 4.25), i.e. immediate transcriptional response, and 24 hrs (day 5) of RA+BMP4 treatment, compared to day 4 (n=3). (B) Venn diagram showing the overlap of significantly upregulated genes (Log fold change >2, FDR <0.01) after 6 hrs and 24 hrs of BMP4+RA treatment. Upregulated genes were identified by two pairwise comparisons (i) day 4 vs day 4.25 (smaller circle) and (ii) day 4 vs day 5 (larger circle); 78 genes were identified as common to both comparisons. (C) GO analysis of the 78 common genes were performed using the DAVID functional annotation tool. The bar plot shows that the Wnt signaling pathway is enriched in all three GO categories: biological processes, molecular processes, and pathway categories. (D) Heat map showing the expression values (FPKM) of 19 Wnt ligands at day 4 (dP state 1), day 4.25 (immediate transcriptional response to BMP4) and day 5 (dP state 2). The expression values of n=3 biological replicates are shown at each timepoint. (E) Volcano plots showing upregulated genes at 6 hrs and 24 hrs of RA+BMP4 treatment. Multiple Wnt ligands and Wnt pathway genes are highly upregulated in both conditions. (F) RT-qPCR validation of selected Wnt genes. mESCs were differentiated up to day 5 under RA±BMP4 conditions and RA+BMP4 along with 24 hrs of Noggin treatment. The expression of the Wnt ligands was significantly upregulated on BMP4 addition, but reduced if noggin, a BMP signaling inhibitor, was added along with the BMP4. N=Ct values of 3 independent differentiations are plotted and data are presented as the mean±SEM. Significance is determined by two-way ANOVA (Tukey's multiple comparison test), *=p<0.05, *p<0 0.005, *** p<0.0005.

FIG. 5. Wnt/β-catenin signaling is required for BMP4-mediated neuronal diversity. (A) Timelines of directed differentiation protocols, to assess the requirement for Wnt signaling in the induction of BMP4 dependent dI fates. IWR1e, a small molecule inhibitor of canonical β-catenin signaling, was added for 48 hours, at day 4. Samples were collected at day 9 for immunohistochemical (IHC) and RT-qPCR analyses. n=3 independent differentiations. (B-Q) Protocol 1 (RA alone) induces Pax2+dI4/6s (E, Q) and Lmx1b+dI5s (E), while protocol 3 (RA+BMP4) induces the Lhx2+dI1s (F, N), Foxd3+dI2s (G, O) and IsI1+dI3s (H, P). The addition of IWR1e to protocol 3 results in the dramatic reduction in the dI1 and dI2 populations (J, K, N, O), a modest increase in dI3s (L, P) and concomitant increases in dI4, dI5 and dI6s (M, Q). Thus, Wnt signaling is required for BMP4 to direct the dorsal most fates during dI differentiation. For qPCR analysis, n=3 independent differentiations. Significance tests: one-way ANOVA, *=p<0.05, **p<0 0.005, *** p<0.0005. Scale bar 100 μm.

FIG. 6. Wnt/β-catenin signaling mediates proliferation, rather than patterning, of mESCs derived spinal NPCs. (A) Timeline of the NMP-based directed differentiation protocol, to assess whether Wnt signaling modulates the BMP-mediated dI fates though patterning activities. Multiple Wnts (Wnt1, Wnt2, Wnt3a, Wnt4, Wnt9b), or CHIR, a general Wnt agonist, were added either individually, or together, for 24 hrs at day 5. Samples were collected at day 9 for either immunohistochemical (IHC) or RT-qPCR analyses, n=2 independent differentiations. (B) RT-qPCR analyses demonstrated that the addition of either Wnts (100 ng/ml) or CHIR (5 μM) has no significant effect (one way ANOVA) on identity of the BMP4-mediated dI fates. Cell fate were assessed by the expression of Lhx2 (dI1), Foxd3 (dI2) and IsI2 (dI3); expression levels were normalized to the day 0 and RA condition. (C) Timeline of the embryoid body (EB) directed differentiation procedure, to assess the effect of activating Wnt signaling on the proliferation of dorsal NPCs. EBs were treated with RA±BMP4, as for protocol 1 and 3, and then treated with either DMSO or CHIR99021 for 4 days, starting at day 5. Samples were collected at day 9 for immunohistochemical (IHC) and RT-qPCR analyses. n=2 independent differentiations. (D-I) The EBs were assessed for the distribution of two mitotic markers: EdU incorporation (D-G; cells in S-phase) and antibodies against phosphorylated histoneH3 (D-G; pH3, M-phase). Quantification of the number of EdU+(H) and pH3+(1) cells (n=15-25 EBs) demonstrated that CHIR treatment increases cell proliferation by ~3 fold in RA±BMP4 patterned EBs, compared to DMSO treatment, resulting in increased numbers of Sox2+NPCs (D-G). Significance was determined using one-way ANOVA (Kruskal-Wallis test). (J-P) The identities of CHIR treated NPCs were assessed using antibodies against Pax3 (J-M; all dPs), Olig3 (J-M; dP1-dP3) and Pax7 (J; dP4-dP6). Both IHC (J-M) and RT-qPCR analyses (N-P) demonstrated that CHIR treatment increases the numbers of NPC cells but does not significantly (unpaired t-test) alter their dorsal-ventral identities. For qPCR analysis, n=3 independent differentiations. Significance values: *=p<0.05, **p<0 0.005, *** p<0.0005. Scale bar=200 μm.

FIG. 7. Activating canonical Wnt signaling expands mESC-derived NPC populations. (A) Schematic for the expansion timeline for mESC-derived spinal cord progenitors. (B−I) RA−(B−D) and RA+BMP4-treated (F-H) EBs were passaged in the presence of DMSO (B, F) or CHIR (C, D, G, H). Bright field images (B, C, F, G) show that passaging with CHIR results in a >6-fold increase in the number of EBs after each passage, compared to DMSO-treated EBs, which rapidly decline after the 1st passage (E, I; one-way ANOVA Kruskal-Wallis test, n=2 independent differentiations). All CHIR-treated EBs maintain a Sox2+NPC identity after passage 1 and 2 (D, H), but the expansion capacity of RA-patterned EBs was less robust than that of RA+BMP4-patterned EBs (E, 1). (J) Schematic of the timeline and experimental approaches taken to assay the differentiation potential of CHIR-treated EBs. The left timeline identifies the conditions needed to induce neural differentiation in CHIR-expanded EBs following their dissociation. The right timeline determines the extent to which dI patterning can be restored in CHIR-treated EBs, with a 2-day pulse of RA±BMP4. (K-Q) Immunohistochemical analyses of passage 6 EBs (48 days) revealed that CHIR treatment sustains Sox2+NPCs at day 10 of differentiation (K, N) while removal of CHIR induces Tuj1+neural differentiation in both RA−(L) and RA+BMP4-treated (O) EBs. The addition of DAPT had a modest effect increasing the number of Tuj1+neurites (M, P). RT-qPCR analyses confirmed that CHIR depletion resulted in increased expression of Tubb3 and Neu, two markers of neural differentiation (Q). However, there was no significant change in the number of Sox2+ cells. (R-X) The patterning profile of the expanded EBs was assessed. Expanded EBs retained some capacity to differentiate into Lmx1b+dI5s (R, X; RA control) or IsI1+dI3s (U, RA+BMP4 control), but lost the ability to generate dI1s, dI2s and d4s (R, T, X). However, a 2-day pulse from day 3-5 of RA±BMP4 restored dI1/2 differentiation (V, X) and resulted in~5-fold increase in the number of Pax2+-dI4/6 neurons (S, X). Patterning potential was assessed for passage (p) 1 and p6 (X), and did not appear to significantly change much over time, other than for the dI2s, where differentiation potential modestly improves over time. N=6-10 EBs were counted for each condition, significance determined by Mann-Whitney test. Significance values: *=p<0.05, **p<0 0.005, ** p<0.0005. Scale bar: B, C, F, G=2 mm, Q−V=100 μm; J-0=2000 μm.

FIG. 8 (related to FIG. 1). Transcriptomic analysis of in vitro differentiation under conditions that direct either dorsal sensory interneurons or cardiac mesoderm identity. (A) Expression analysis for Sox2 and T at day 2 and day 3 of the protocol. After a 48 hr exposure to bFGF alone, cells upregulate Sox2 but not T by day 2. Providing another 24 hr pulse of both bFGF and CHIR induce the expression of Sox2 and T, the signature of neuromesodermal progenitor (NMP) identity by day 3 of the protocol. (B, C) Principal component (PC) 1 and 2 separates the undifferentiated and differentiated samples in all three tested conditions (R, B, and C). PC3 and 4 separates the neural and cardiac mesodermal conditions. (D) The cellular component category of the GO analysis of the C-branch is enriched for muscle-related terms, supporting a cardiac mesoderm identity. (E) Summary of all eigengene modules identified in the weighted gene co-expression analysis (WGCNA) for all three conditions. For each eigengene module, the top GO category is displayed. (F) The day 9 cells in the C-branch (C9; protocol 2) upregulate endocardium specific genes, while B9 cells (protocol 3) upregulate dorsal spinal cord-specific genes. Scale bar=100 μm.

FIG. 9 (related to FIG. 2). Characterization of the neuronal populations derived under RAt BMP4 conditions by single-cell sequencing and immunohistochemistry. (A, B) The UMAP and feature plots identify clusters of stressed cells, pluripotent cells, and mesodermal/neural crest (NC) cells in day 9 samples of both protocols 1 (A, RA) and 3 (B, RA+BMP4). Stressed cells are identified by low RNA content (<2000) in the nFeature plots.

The feature plots show the expression of both pluripotent markers—Sox2, Nanog, and Pou5f1 (Oct4) and mesodermaVNC markers—Twist1, Runx2, and Foxc2. (C-D) The protocol 1 feature plots contain mostly progenitors and interneurons specific to the intermediate spinal cord (dP4-dP6, dI4-dI6). In contrast, the protocol 3 feature plots are enriched for the expression of the dorsal-most spinal identities (dI1-dI3, dP1-dP2). (E, F) Six classes of dIs can be identified in transverse sections of E11.5 thoracic spinal cord in mouse embryos, using the antibodies directed against Lhx2 (upper edges of image in E), Foxd3 (lower clusters and lateral regions of positive cells in E). Pax2 (medial region of positive cells in F). Lmx1b (brightest positive cells in F) and IsI1 (lateral regions of positive cells in F). (G, H) Both immunohistochemical (IHC) staining and quantitative RT-PCR analyses at day 9, validate that the dI1-dI3 populations are derived in protocol 3, while the dI4-dI6 populations are induced in protocol1. For the qRT-PCR assays, significance was determined using an unpaired t-test (n=3), for the IHC significance was determined using the Mann-Whitney test (n=~10 images from 2 differentiations). Scale bar=100 μm.

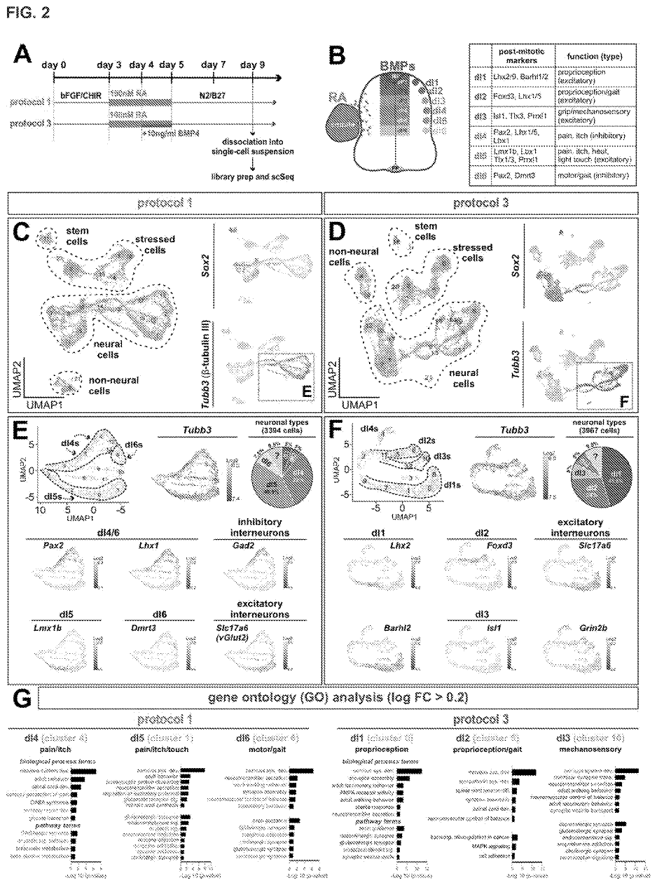

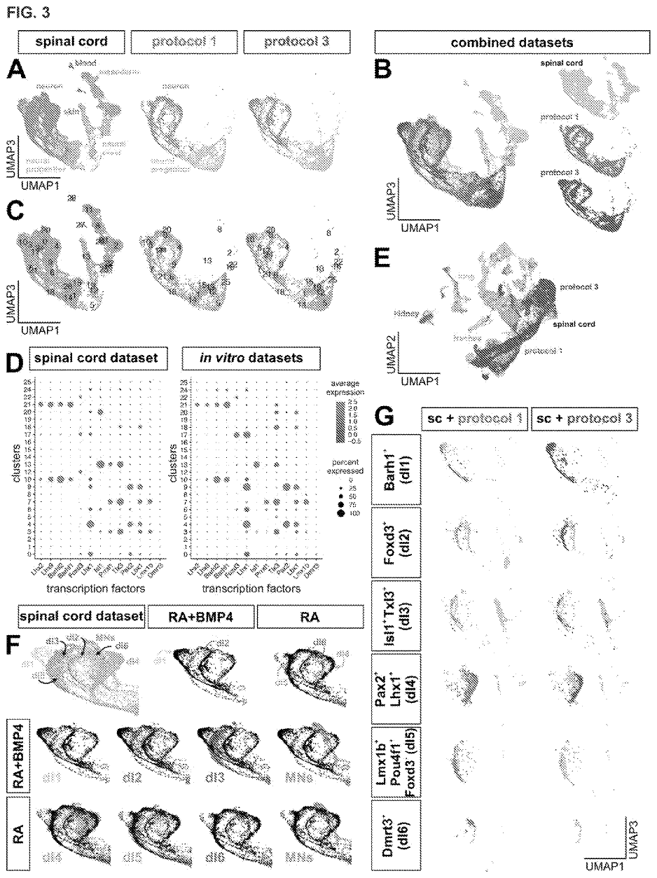

FIG. 10 (related to FIGS. 2 and 3). Identification of novel markers in distinct dI populations. (A) Combined UMAP plot of Tubb3+ neurons derived from protocol 1 and protocol 3, reveals six expression domains for dI1-dI6s (dI1: BarhI2, dI2: FoxD3, dI3: IsI1, dI4: Pax2, dI5: Lmx1b, dI6:Dmrt3). (B) Novel dI-specific markers were identified by differential expression analysis of the combined Tubb3+ clusters on Seurat. These markers include ion channels (Kctd4, Kcnk9, Grial), cell-surface proteins (Cd44, Cacna2d3), transcription factors (Skor1, Hmx2, Tox2), receptors (Ntrkl), adhesion molecules (Cntn3, Cntnap5a, Chl1). (C) UMAP plot depicting Tubb3+ neurons extracted from the scRNA-Seq spinal cord. Neuronal identities were assigned according to the published annotations (Delile et al., 2019). (D) Feature plots comparing the expression of various neuropeptides in in vivo- and ESC-derived dIs. Neuropeptides are expressed in the mature dIs and regulate the intensity of sensory signals in the adult spinal cord. The similarities between the neuropeptide expression profiles suggests that ESC-derived dIs follow a similar maturation trajectory to the embryonic spinal cord.

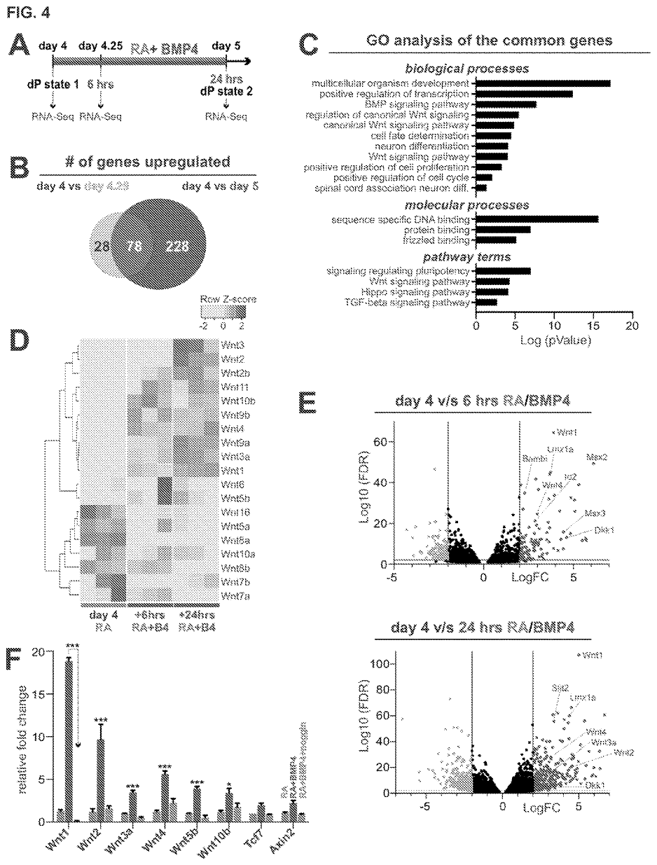

FIG. 11 (related to FIG. 4). Global transcriptional changes induced by RA, which drive mESCs towards dI4-6 fates. (A, B) Timelines of directed differentiation protocols 1 and 3 (A), and the sample collection for bulk RNA-Seq analysis of protocol (B) to investigate the transcriptomic changes induced by 6 hrs (day 3.25), i.e. immediate transcriptional response, and 24 hrs (day 4) of RA treatment, compared to day 3, (n=3). (C, D) Volcano plots showing the differentially expressed genes (Log fold change>2, FDR<0.01) at 6 hrs and 24 hrs of RA incubation as compared to the NMP state (day 3). (E) Heat map showing the expression (FPKM values) of genes differentially expressed day 3 (NMP state), day 3.25 (immediate transcriptional response to RA), and day 4 (dP state 1). In total, the expression levels of 979 genes are altered, with 389 genes upregulated, and 590 genes downregulated. (F, G) Bar plots showing the top twelve GO categories of the differentially expressed genes either 6 hrs (F) or 24 hrs (G) after treatment with RA, compared to the NMP state (day 3). GO terms were obtained using the Metascape analysis tool. (H, 1) Protein-protein interaction (PPI) network plots for the most significantly upregulated genes after 6 hrs (H) or 24 hrs (1) of RA treatment. By 6 hrs of RA treatment, a nascent PPI network forms with 3 nodes, including Meis1 and Meis2 (H). However, by 24 hrs in RA (1), the PPI network expands substantially to include multiple nodes with MCODE modules that are relevant to both spinal cord development and sensory neuron fate specification. In particular, Meis1 and Meis2 are now present in a PPI network that contains genes such as Pax6, Lmx1b, Lbx1, Dbx1 and Msx1 that are expressed in the intermediate spinal NPCs and thought to regulate dI4-dI6 development. Other significantly enriched PPI modules include sensory organ morphogenesis, which contains genes such as Irx1, Irx3 and Irx5, that are also expressed in the intermediate spinal cord. (J) Heat map showing the expression (FPKM values) of 911 genes upregulated by a 24 hr treatment of 100 nM RA on day 4, compared to the day 4 control condition (no RA treatment). These genes are not upregulated, if AGN193109, a pan-RAR inhibitor, is added in 10 molar excess with the RA. The RA-induced genes include Meis1, Meis2, Pax6, Lmx1b, Lbx1 and Dbx1 i.e the genes that establish the patteming PPI networks in panel H and I.

FIG. 12 (related to FIG. 4.: RA induced gene expression changes in NMPs. (A) Timeline of Pax3 and T (brachyury) expression after RA treatment starting at day 3. Pax3 expression increases with RA exposure, while T expression rapidly decreases. (B) Immunohistochemical analyses confirms that levels of Pax3 increase at day 4 compared to day 3, i.e. after 24 hrs of RA exposure. (C) The metascape algorithm identifies13 functional MCODE modules in the PPI network which was upregulated by the 24 hr pulse of RA exposure at day 4. Note that the modules related to sensory organ development and pattern specification process contain genes that are known to be expressed in the developing intermediate spinal cord. (D) qRT-PCR validation of selected genes in the pattern specification and sensory organ development modules. Both Meis1 and Meis2, but not Meis3, are induced by the addition of RA; both genes form the core of the pattern specification module (FIG. 3(I)). Significance was determined using one way ANOVA (n=2). (E-F) RA and BMP4 act sequentially in dI specification: 6 hr pulse of of RA at day 3 is sufficient to direct NMPs towards a Pax2+dI4/dI6 fate by day 9 (E). In contrast, the Lhx2+dI1fate requires a 24 hr pulse of RA, followed by the addition of BMP4 at day 4. Scale bar=50 μm.

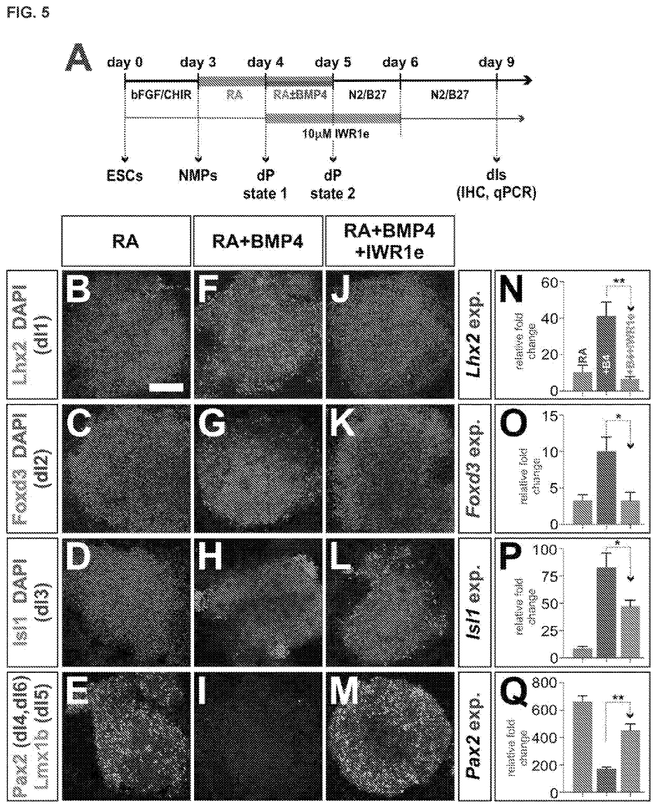

FIG. 13 (related to FIGS. 4 and 5). BMP4 induced gene expression changes and the effects of Wnt inhibition on dI identity. (A) Heatmap showing the expression (FPKM) of all significantly upregulated genes after 6 hrs and 24 hrs of RA+BMP4 exposure, as compared to the RA-alone condition at day 4. (B) The expression (FPKM) of 78 genes upregulated at both 6 and 24 hrs after RA+BMP4 exposure, as compared to day 4 RA-alone. Many Wnt ligands are included in this gene set. (C) Heatmap showing the dynamic expression of Wnt receptors after 6 and 24 hrs of RA+BMP4 exposure, compared to day 4 RA-alone. (D-E) Inhibiting Wnt/β-catenin signaling, through either IWR1e or XAV939, most strongly suppresses the differentiation of the BMP4-dependent dI1/dI2s, with a more modest effect on dI3s (D). Concomitantly, there is an increase in the expression of dI4-dI6 specific genes, suggesting that BMP4 requires Wnt signaling to suppress dI4-6 identity. (F) Both immunohistochemical and qRT-PCR analyses demonstrate that either IWR1e or XAV939 treatment prevents both the suppression of Pax7+dPs and induction of Olig3+dPs, observed in the RA+BMP4 condition. Statistical significance was determined using two-way (D, E) or one-way (F) ANOVA (N=2 independent experiments). Scale bar=100 μm.

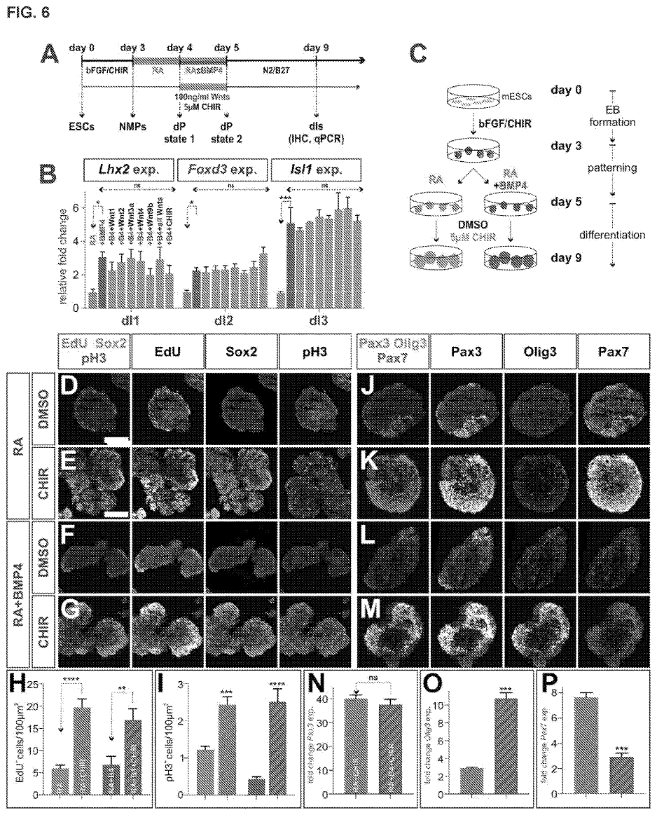

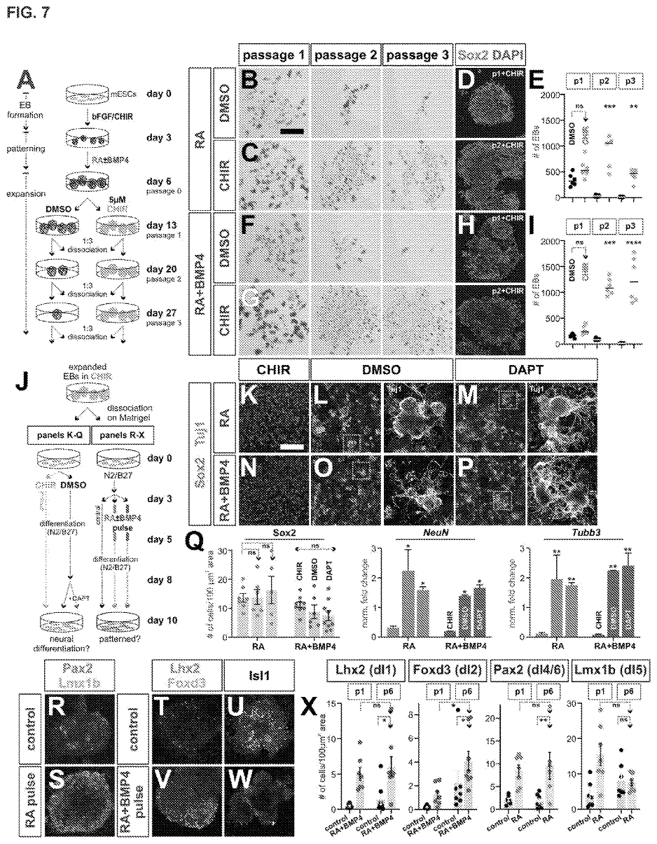

FIG. 14 (related to FIGS. 6 and 7). Assessing the sufficiency of Wnt ligands for dI fates and the effect of passaging on EB size and dI differentiation. (A) RT-qPCR analysis demonstrate that neither canonical Wnts (Wnt1, Wnt2, Wnt3a, Wnt4, all at 100 ng/ml) nor a non-canonical Wnt ligand (Wnt9b) induce the BMP4 dependent dI fates when added at day 4 for 24 hrs. Combining all Wnt ligands together or activating Wnt signaling using 5 μM CHIR also do not induce BMP4-dependent dI fates. Cell identity was determined by the expression of Lhx2 (dI1), Foxd3 (dI2), IsI1 (dI3) at day 9 of the differentiation procedure. Ct values were normalized to RA-alone to calculate the fold change at day 9. (B-C) Quantification of EB size over three consecutive passages (p), cultured in either DMSO or CHIR. CHIR-induced proliferation causes a decrease in EB size, but higher EB numbers (FIG. 7B-7G). The effect of passaging is similar in both RA (B) and RA+BMP4 patterned (C) EBs. (D, E). The proportion of Sox2+ cells does not change over time. Rather, the Sox2 negativity at center of the EBs is a result of these cells dying, probably due to lack of nutrient support. The images in (D) are taken from RA±CHIR EBs. (F) Schematic of the developing spinal cord showing that Pax3 is expressed in dP1-dP6, while Pax7high and Olig3 mark the intermediate (dP4-dP6) and the dorsal most (dP1-dP3) progenitor domains respectively. (G, H) By p6, RA-patterned EBs retain Pax3+ Pax7+ intermediate spinal cord progenitor identity. In contrast, the dorsal identity of RA+BMP4-patterned EBs has eroded, such that they also have an intermediate spinal identity. (G) By p6, RA-patterned EBs still produce Lmx1b+dI5s efficiently, but not Pax2+dI4/dI6s. RA+BMP4-patterned EBs lose the ability to generate dI1/dI2s and also generating Lmx1b+dI5s. Significance was determined using one-way ANOVA (n=2) (A) the Kruskal-Wallis test (B, C) (n=no. of EBs shown on the plots), two-way ANOVA (E), the Kruskal-Wallis test (n=2 differentiations) (H, J). Scale bar=100 μm.

FIG. 15. Schematic of dI differentiation from hPSCs through NMP intermediate.

FIG. 16. Formation of embryoid bodies from NMP monolayer. (A) By day 6 in N2/B27 (-VitA) media, supplemented with FGF and Wnt agonist CHIR99021, the hPSCs have been converted into NMPs and have formed a tight monolayer. (B) Embryoid bodies are formed by the EZ passage tool which is used to cut the NMP monolayer on day 6, resulting in square-shaped cell clusters. (C) Embryoid bodies are formed by day 7 after these clusters are transferred to an ultra-low attachment plate in N2B27 media (+VitA). Scale bar: 500 μm.

FIG. 17. Immunostaining of D36 embryoid bodies to detect dI4-6 in protocol 2.1 and dI1-3 neurons in protocol 2.2. By day 36, EBs can be fixed, sectioned, and immunostained for dI specific markers. (A) The EBs derived from Protocol 2.1 will contain pain, itch and heat relaying Pax2+ Tuj1+dI4-dI6 neurons (33%) and Pax2−ve, Lmx1b+ve dI5s (30%). (B) In contrast, the EBs derived from Protocol 2.2 contain a mixture of proprioceptive Lhx2+dI1s (40%), Foxd3+dI2 (20%) and mechanosensory IsI1+Tuj1+dI3s (10%). Scale bar: 100 μm.

FIG. 18. Functional assay of sensory EBs using calcium sensors. (A) Strategy to tag control and patient's iPSC-derived sensory EBs with adenovirus containing calcium sensor GCaMPf6. EBs are imaged on 2 photon microscope after 2 weeks of infection, after stimulation by 100 uM Kainic acid to induce spontaneous activity. (B) Example of neuronal activity in sensory EBs identified by monitoring calcium-GFP currents in day 35 EBs (left). Calcium traces show spontaneous firing of neurons (right), indicating functional dis in EBs.

### DETAILED DESCRIPTION

The methods described herein are based on the surprising discovery that it is possible to create a full complement of spinal sensory interneurons from mouse embryonic stem cells (ESCs). These ESC-derived and endogenous dis are transcriptionally indistinguishable. The same protocol, with adjustments for timing, can be used with human cells. The methods described herein provide a disease model for sensory processing disorders, such as fibromyalgia, and autism. Cells can be obtained from an individual subject using a skin biopsy to develop the stem cells that can then be used for study and testing that is tailored to the individual subject.

Provided are new methods for producing a complete atlas of in vitro-derived dIs. For the first time, all known classes of dIs can be derived from stem cells. Moreover, these in vitro-derived dIs resemble their in vivo counterparts to a remarkable degree. This provides a suitable model system for further mechanistic and translational studies. This atlas can be used to screen and develop novel analgesics or study the mechanistic action of drugs such as cocaine and amphetamine on spinal neural circuitry at the cellular level.

The current understanding of dorsal spinal cord development remains surprisingly incomplete, despite its central role in mediating sensory information. While some progress has been made, this problem remains unresolved because the number of signaling factors in the dorsal spinal cord have made genetic approaches in vivo especially challenging. The in vitro recapitulation of the dorsal spinal cord now permits new insights into unresolved developmental mechanisms. First, we reveal the hierarchical nature of fate specification decisions mediated by RA and BMP signaling. The concomitant application of RA and BMP4 to neuromesodermal progenitors (NMPs) leads to the specification of cardiac mesodermal lineages. Only the sequential application of RA, followed by BMP4, leads to the dorsal most spinal fates. The data described herein further show that RA induces a default dorsal progenitor state (dP state 1), which resolves into the dI4-dI6 fates. The addition of BMP4 to dP state 1 then induces a second multipotential fate (dP state 2), which resolves into the dI1-dI3 fates.

Second, this study resolves the controversy surrounding the role of canonical Wnt signaling in spinal cord development. We unambiguously show that Wnts act as mitogens, not morphogens. In a further important finding: we use this mechanistic insight to design a novel method for deriving dIs in large quantities without changing their identity. This insight will be key to generating enough dIs for drug screening and clinical purposes, and is likely to be relevant for expanding other populations of spinal neurons, such as motor neurons and ventral interneurons.

We have used our single cell atlas to identify and characterize additional dI markers.

First, we have identified novel markers for specific dI classes, which include receptors, ion channels and adhesion molecules. These markers are detailed in Table 4, and FIG. 10A, 10B. We also assessed the distribution of neuropeptides, including neuropeptide Y, neuromedin S, prepronociceptin and gastrin releasing peptide in our in vitro derived dIs as a marker of their maturation. We find that some neuropeptides show specificity to particular dI types, and that the distribution of neuropeptides are conserved between the in vitro and in vivo data sets (FIG. 10C, 10D). Together, this data further supports the hypothesis that the in vitro derived neurons can mature into diverse sensory subtypes, in an equivalent manner to endogenous dIs.

### Definitions

All scientific and technical terms used in this application have meanings commonly used in the art unless otherwise specified. As used in this application, the following words or phrases have the meanings specified.

As used herein, “neuromesodermal progenitor” (NMP) refers to a cell that is bipotential for both neural and mesodermal fates, and characterized by co-expression of both mesodermal (Brachyury (T)) and neural progenitor cell (NPC, Sox2) markers.

As used herein, “neural progenitor cell” (NPC) refers to a cell that is immunopositive for nestin, capable of continuous growth in suspension cultures and, upon exposure to appropriate conditions, can differentiate into neurons or glial cells.

As used herein, “dorsal neural progenitor cells (NPCs)”, “dorsal NPCs”, or “dNPCs” refers to Pax3+Sox2+ cells. Dorsal NPCs can be further identified as Type 1 and Type 2. Type 1 dNPCs are induced by contact with retinoic acid, and express the markers Pax3, Pax7, Sox1, and Sox2. Type 2 dNPCs are induced by treatment with retinoic acid and BMP4, and express the markers Pax3, Olig3, and Sox2.

NPCs can be generated in vitro by differentiating embryonic stem cells or induced pluripotent stem cells (iPSC). iPSCs are derived from adult cells, most often from fibroblasts or blood cells, and programmed into an embryonic-like pluripotent state.

As used herein, to “maintain a dorsal spinal intemeuron fate potential” or “maintain dI fate potential” refers to the ability of the referenced cells to respond to a pulse of RA or RA+BMP4 (per the differentiation protocols described herein) by adopting a dI1-dI6 phenotype.

As used herein, a “dI1-dI6 phenotype” refers to dIs that express one or more characteristics that distinguish dorsal spinal interneurons from other cell types. Within the six subtypes of dIs, dI1-dI6, the cell may express a phenotype specific to a corresponding dI, e.g., dI1, dI2, dI3, dI4, dI5, or dI6. For example, expression of the following genes is indicative of the corresponding specific dI1-dI6 phenotypes: dI1=BarhI1; dI2=Foxd3; dI3=TIx3 and Is1l; dI4=Pax2 and Lhx1; dI5=Lmx1b and Pou4f1; and dI6=Dmrt3. Additional examples of markers of specific dI1-dI6 phenotypes are listed in Table 4.

As used herein, a “control” or “reference” sample means a sample that is representative of normal measures of the respective marker, such as would be obtained from normal, healthy control subjects, or a baseline amount of marker to be used for comparison. Typically, a baseline will be a measurement taken from the same subject or patient. The sample can be an actual sample used for testing, or a reference level or range, based on known normal measurements of the corresponding marker.

As used herein, a “significant difference” means a difference that can be detected in a manner that is considered reliable by one skilled in the art, such as a statistically significant difference, or a difference that is of sufficient magnitude that, under the circumstances, can be detected with a reasonable level of reliability. In one example, an increase or decrease of 10% relative to a reference sample is a significant difference. In other examples, an increase or decrease of 20%, 30%, 40%, or 50% relative to the reference sample is considered a significant difference. In yet another example, an increase of two-fold relative to a reference sample is considered significant.

As used herein, the term “subject” includes any human or non-human animal. The term “non-human animal” includes all vertebrates, e.g., mammals and non-mammals, such as non-human primates, horses, sheep, dogs, cows, pigs, chickens, and other veterinary subjects. In a typical embodiment, the subject is a human.

As used herein, “a” or “an” means at least one, unless clearly indicated otherwise.

### Methods & Models

The methods described herein provide novel methods for deriving dorsal interneurons (dIs) of all types, dI1-dI6, as well as methods for producing dIs in large quantities without changing their identity, and methods for using same. In some embodiments, the method for producing dorsal spinal interneurons (dI) in vitro comprises contacting a population of neuromesodermal progenitor cells (NMPs) with retinoic acid (RA) for a sufficient time to neuralize the NMPs into dorsal neural progenitor cells (NPCs); and culturing the dorsal NPCs with retinoic acid (RA) for an additional day, and optionally, further contacting the dorsal NPCs with bone morphogenic protein 4 (BMP4) for the additional day. The contacting with RA is performed in the absence of BMP4. Typically, a sufficient time to neuralize the NMPs is about one day of contact with RA in the absence of BMP4. NMPs can be derived from embryonic stem cells (ESCs) by contacting the ESCs with basic fibroblast growth factor (bFGF) and an activator of the Wnt/beta-catenin pathway, such as CHIR, typically for about 3 days. In some embodiments, the ESCs are contacted with bGFG for two days, followed by one day of contact with both bFGF and CHIR.

In some embodiments, the method of producing dIs comprises contacting a population of dorsal neural progenitor cells (NPCs) with an activator of the Wnt/beta-catenin pathway; and culturing the dorsal NPCs of (a) under conditions permitting proliferation, wherein the dorsal NPCs maintain their dI fate potential. In some embodiments, the activator of the Wnt/beta-catenin pathway is CHIR (C₂₂H₁₈Cl₂N₈; CAS No. 252917-06-9). In some embodiments, the dorsal NPCs are obtained by contacting a population of NMPs with retinoic acid (RA) for about two to about 20 days. In some embodiments, the contacting with RA continues for up to ten days. The contact with the activator of the Wnt/beta-catenin pathway is made with cells that are NPCs, rather than ESCs, to maintain the dI fate potential. Activation of the Wnt signaling with ESCs diverts stem cells towards the mesodermal fate when the ESCs have not yet been treated with retinoic acid (RA). In some embodiments, the contacting with RA is for about two days. In some embodiments, the contacting with RA is sufficient to direct the NMP cells to a NPC state. For human cells, contacting with RA for 4-6 days is typically sufficient to induce an NPC state. In some embodiments, the contacting further comprises contacting with bone morphogenic protein 4 (BMP4) for the second of the two days, or the latter 2-3 days with human cells. In some embodiments, the contacting with BMP4 continues for up to 10 days (e.g., the latter portion of the contacting with RA for, e.g., 20 days). In some embodiments, the contacting with BMP4 is for about 24 hours. In some embodiments, the contacting with BMP4 is for about 2-3 days.

In some embodiments, the method further comprises subsequently ceasing the contacting with an activator of the Wnt/beta-catenin pathway, followed by contacting the dorsal NPCs with retinoic acid (RA) for about two days, optionally for up to 20 days. In some embodiments, the contacting with an activator of the Wnt/beta-catenin pathway occurs for up to 10 days. In some embodiments, the NPCs are further contacted with bone morphogenic protein 4 (BMP4) for the second of the two days, and, optionally, for up to 10 days (e.g., the second half of a 20 day protocol). In some embodiments, the dorsal NPCs comprise a plurality of each of dI1, dI2, dI3, dI4, dI5, and dI6 phenotypes. In some embodiments, the dorsal NPCs contacted with RA express Pax3 and Pax7, and the dorsal NPCs contacted with RA and BMP4 express Pax3 and Olig3. In some embodiments, the dorsal NPCs express one or more dI markers listed in Table 4. In some embodiments, the activator of the Wnt/beta-catenin pathway (e.g., CHIR) is withdrawn. Ceasing the contact with the activator of the Wnt/beta-catenin pathway induces differentiation with mixed dI identities. Treatment of the NPCs post-CHIR withdrawal with RA and with RA+BMP4 is used to direct the differentiation to the desired dI types from NPCs.

In some embodiments, the population of dis produced by the method described herein comprises at least 5% dI1, dI2, dI3, dI4, dI5, and/or dI6, wherein at least 5% of the cells in the population are of the indicated type of dI. In some embodiments, the population of dis produced by the method described herein comprises at least 10% of dI1, dI2, dI3, dI4, dI5, and/or dI6. In some embodiments, the population of dIs produced by the method described herein comprises at least 5% of each of dI1, dI2, dI3, dI4, dI5, and dI6. In some embodiments, the population of dIs produced by the method described herein is enriched for one or more types of dIs 1-6. In some embodiments, the method described herein produces dI2 and/or dI5 interneurons.

In some embodiments, the culturing of NMP is three-dimensional culturing. Three-dimensional culturing is used to obtain embryoid bodies (EBs), which have been shown to form functional sensory circuits. These EBs provide an ex vivo model for disorders affecting sensation. In some embodiments, the NMPs are obtained from a population of induced pluripotent stem cells (iPSCs). The iPSCs can be obtained from a subject used to direct patient-specific EBs containing either healthy or mutant dIs. These new classes of protocols also provide an unlimited source of bonafide human dIs for cellular replacement therapies to reestablish sensory connections in injured patients.

Also described herein is a method of screening neuroactive agents. In some embodiments, the method comprises contacting a population of dorsal spinal interneurons (dIs) produced by the above method with a candidate neuroactive agent; and measuring a change in physicochemical properties of the dIs relative to a reference population of dIs, wherein a neuroactive agent is identified when a change relative to the reference population of dIs is measured. In some embodiments, the candidate neuroactive agent is an analgesic, and the population of dIs is enriched for dI4 and/or dI5. In some embodiments, the population of dIs has been enriched for dI4 and dI5 by contacting the dorsal NPCs with retinoic acid (RA) for about two days, and subsequently culturing the dorsal NPCs in the absence of growth factor. In some embodiments, the candidate neuroactive agent is a psychoactive compound. In some embodiments, the neuroactive agent is cocaine or amphetamine. In some embodiments, the dI are murine or human dI. In some embodiments, the screening is performed in vitro or ex vivo.

The types of dIs and their associated functions are as follows: dI1—proprioception; dI2—proprioception/gait stabilization; dI3—touch/motor control (grip strength); dI4-pain/itch; dI5—touch/pain/itch; and dI6—motor control. Thus each population of dIs is tuned to a different sensory modality (or modalities) and each one represents a different potential drug screening target depending on the objective (i.e. looking to suppress pain, itch, or to alter balance). One can screen for a variety of agents using any one or a combination of dis, selected based on the desired objective, e.g., in screening for compounds that specifically activate or suppress neural activity in the different dIs.

Also provided is a method of transplanting dorsal NPCs to the spinal cord of a subject in need thereof. In some embodiments, the method comprises administering dorsal NPCs produced by one of the methods described herein to the subject. In some embodiments, the dorsal NPCs are administered by injection to the spinal cord of the subject. The dIs or dorsal NPCs produced by the methods described herein provide a means of cellular therapy to replace diseased or damaged spinal tissues, and thereby restore sensory modalities.

Restoration of some or all of the missing sensory modalities can be achieved by selecting particular dIs (or dPs) to be transplanted.

### EXAMPLES

The following examples are presented to illustrate the present invention and to assist one of ordinary skill in making and using the same. The examples are not intended in any way to otherwise limit the scope of the invention.

### Example 1: In Vitro Atlas of Dorsal Spinal Interneurons Reveals Wnt Signaling as a Critical Regulator of Progenitor Expansion

Restoring sensation after injury or disease requires a reproducible method for generating large quantities of bona fide somatosensory interneurons. Towards this goal, this Example assesses the mechanisms by which dorsal spinal interneurons (dI1-dI6) can be derived from mouse embryonic stem cells (mESCs). Using two developmentally relevant growth factors, retinoic acid (RA) and bone morphogenetic protein (BMP) 4, we have recapitulated the complete in vivo program of dI differentiation through a neuromesodermal intermediate. Transcriptional profiling reveals that mESC-derived dIs strikingly resemble endogenous dis, with the correct molecular and functional signatures. We further demonstrate that RA specifies dI4-dI6 fates through a default multipotential state, while the addition of BMP4 induces dI1-dI3 fates and activates Wnt signaling to enhance progenitor proliferation.

Constitutively activating Wnt signaling permits the dramatic expansion of neural progenitor cultures. These cultures retain the capacity to differentiate into diverse populations of dIs, thereby providing a method of increasing neuronal yield.

Spinal cord injuries (SCIs) can result in the loss of both coordinated movement and somatosensation (lonta et al., 2016; Lenggenhager et al., 2012), and the ability to perceive the environment. Without somatosensation, patients can no longer communicate through touch, sense danger through pain, or control the functions of the gut and bladder (Furusawa et al., 2011). While stem cell-based cellular replacement therapies have shown promise for SCI (Antonic et al., 2013: Kong et al., 2021), the focus so far has been on the recovery of coordinated motor function (Faravelli et al., 2014; Trawczynski et al, 2019). Less attention has been paid to regaining sensory functions, despite their critical roles ensuring physical and emotional well-being, and providing modulatory feedback to the motor system through proprioceptive pathways. Somatosensory information is received peripherally and then relayed centrally by different populations of dorsal interneurons (dIs) in the spinal cord. Each dI population is specialized for distinct somatosensory modalities, and forms microcircuits that span different layers of the dorsal horn (Gupta and Butler, 2021; Lai et al, 2016). For example, dI1s (proprioceptors) (Lai et al., 2011), dI2s and dI3s (both mechanosensor) (Bui et al., 2013; Haimson et al., 2020) and dI6 (gait) (Perry et al., 2019) are located in the deep layers of the dorsal horn, while dI4s and dI5s regulate pain, heat, and itch in the superficial layers of the dorsal horn (Koch et al., 2018). To replace damaged spinal tissue, it will be critical to derive a complete complement of dorsal sensory interneurons that molecularly and functionally phenocopy endogenous dIs.

Towards this goal, we have been working to first, unravel the developmental mechanisms by which sensory dorsal interneurons (dIs) arise in the spinal cord, and second, develop directed differentiation protocols to derive dIs from stem cells (Andrews et al., 2017; Gupta et al., 2018; Gupta et al., 2021). Distinct classes of dis arise during embryonic development, when growth factors, including retinoic acid (RA) and the bone morphogenetic proteins (BMPs), pattern six progenitor domains (dP1-dP6), which subsequently differentiate into the dI1-dI6s (Andrews et al., 2019: Gupta and Butler, 2021). The addition of RA and BMP4 to three dimensional embryoid body (EB) protocols (Wichterle et al, 2002), can induce some dI fates in both mouse and human stem cell cultures (Andrews et alt, 2017; Duval et al., 2019; Gupta et al., 2018). However, it has remained unclear how the addition of RA and BMP4 specifically induces dI fates, and whether the dIs generated from these protocols mirror their endogenous counterparts BMPs pattern multiple organ systems throughout development, including the non-neuronal cardiac mesoderm (Kattman et al., 2011; Ladd et al., 1998) as well as osteogenic tissues (Kawaguchi et al., 2005). The BMPs also have reiterative activities specifying dorsal spinal identity, both patterning dPs and controlling their proliferation to ensure that the precise number of specific dis are generated (Andrews et al., 2017; IIIe et al., 2007). While the Smad second messenger complex has a role mediating dI patterning (Hazen et al., 2012; Le Dreau et al., 2012) the downstream signals regulating proliferation remain unresolved.

Here, we define new RA±BMP4 protocols that generate mouse embryonic stem cell (mESC)-derived dIs via a neuromesodermal progenitor (NMP) intermediate (Gouti et al., 2014) that faithfully mimic the normal developmental programs of the neural tube (Delile et al., 2019: Rodrigo Albors et al., 2018; Sagner et al., 2018). This approach has successfully generated a complete atlas of in vitro-derived dIs. Transcriptional profiling of these dis demonstrated that they possess cell fate and sensory function-specific gene signatures that strikingly resemble endogenous dis, indicating that our protocols generate bona fide dIs. Using these protocols as a novel model for dorsal spinal cord development, we identified the hierarchy of fate specification decisions mediated by RA and BMP signaling. RA/BMP signaling directs a series of nested decisions, first between the dorsal spinal cord and cardiac mesodermal fates, and then between two multipotential dP states that direct either the dorsal most (dI1-dI3) or intermediate (dI4-dI6) identities. Through these analyses, we identify Wnt/β-catenin signaling as an immediate downstream response to BMP signaling which maintains neural progenitors in a mitotic state. Remarkably, further elevating Wnt signaling, using the CHIR99021 (CHIR) agonist, can dramatically extend the proliferative capacity of dPs, while preserving their ability to differentiate into specific sensory neurons on demand.

Taken together, this study provides a mechanistic understanding of the developmental trajectories needed to generate the full complement of different dI identities. These insights were used to develop a protocol to expand the propagation of multipotential dPs, thereby paving the way for the production of in vitro-derived dIs needed for both drug screening and cellular transplantation therapies.

RESOURCE TABLE:

| Reagents | Source | Identifier/Catalogue |
|---|---|---|
| | | number |
| Antibodies | | |
| Goat polyclonal anti-Lhx2 | Santa Cruz | Sc-19344 |
| Goat polyclonal anti-Isl1 | R&D Systems | AF1837 |
| Rabbit polyclonal anti-Pax2 | Invitrogen | 71-6000 |
| Mouse monoclonal anti-Lhx1/5 | Developmental Studies Hybridoma bank | 4F2 |
| Goat polyclonal anti-Pax3 | R&D Systems | AF2457 |
| Mouse monoclonal anti-Pax7 | Developmental Studies Hybridoma bank | AB 528428 |
| Rabbit monoclonal anti- | Cell Signaling Technology | D5810 |
| phosphoSMAD1/5 | | |
| Rabbit polyclonal anti-phospho | Cell signaling Technology | 9701 |
| Histone H3 | | |
| Mouse monoclonal Tubulin III | Covance | MMS-435P |
| beta (clone-) | | |
| Goat polyclonal anti-Sox2 | Santa Cruz | SC-17320 |
| Rabbit polyclonal anti-Pax6 | MBL life science | PD-022 |
| Goat polyclonal anti-T | R&D Systems | AF-2085 |
| (Brachyury) | | |
| Guinea pig polyclonal anti- | gift from Thomas Muller; (Muller et al., | |
| FoxD3 sera | 2005) | |
| Guinea pig polyclonal anti- | gift from Thomas Muller; (Muller et al., | |
| Lmx1b sera | 2005) | |
| Guinea pig polyclonal anti- | gift from Thomas Muller; (Muller et al., | |
| Olig3 sera | 2005) | |
| Chemicals, reagents and | | |
| peptides | | |
| CHIR99021 | Tocris Bioscience | 4423 |
| IWR1e | Millipore | 681669 |
| XAV939 | Stem Cell Technologies | 72674 |
| Matrigel membrane matrix | Fisher Scientific, Corning | CB-40234 |
| DAPT | Sigma Aldrich | 565784 |
| Human recombinant BMP4 | Thermo Fisher Scientific | PHC9534 |
| Retinoic acid | Sigma Aldrich | R2625 |
| Human recombinant Wnt1 | Thermo Fisher Scientific | PHC1804 |
| Human recombinant Wnt2 | Millipore | SRP-6560 |
| Human recombinant Wnt3a | R&D Systems | 5036-VVN-010 |
| Mouse recombinant Wnt4 | R&D Systems | 475WN005 |
| Human recombinant Wnt5b | R&D Systems | 7347-WN |
| Mouse recombinant Wnt9b | R&D Systems | 3669-WN-025 |
| Human basic FGF (bFGF) | Thermo Fisher Scientific | PHG0023 |
| DMEM:F12 | Hyclone | SH30023 |
| Neurobasal medium | Thermo Fisher Scientific | 21103049 |
| B27 supplement | Fisher Scientific | 17-504-044 |
| N2 supplement | Thermo Fisher Scientific | 17502048 |
| Mouse LIF | Millipore | ESG1107 |
| Trypsin Inhibitor 1X solution | Sigma-Aldrich | T7659 |
| Paraformaldehyde 16% | Sigma-Aldrich | 28906 |
| Phosphate buffer saline (PBS) | Hyclone | SH30028.02 |
| 1X | | |
| Prolong Gold | Thermo Fisher Scientific | P36930 |
| Critical Commercial Assay | | |
| RNeasy plus mini kit | Qiagen | 74106 |
| Superscript IV First-Strand | Thermo Fisher Scientific | 18091050 |
| synthesis kit | | |
| Dead cell removal kit | Miltenyi Biotec Inc. | 130-090-101 |
| EdU labelling assay | Invitrogen | C10338 |
| Qia shredder | Qiagen | 79654 |
| Softwares | | |
| Corel DRAW version 20 | Corel DRAW | www.coreldraw.com |
| Zeiss Blue lite | Zeiss | https://www.zeiss.co |
| | | m/microscopy/us/pro |
| | | ducts/microscope- |
| | | software/zen-lite.html |
| Graphpad Prism 9 | Graphpad | https://www.graphpad |
| | | .com/scientific- |
| | | software/prism/ |
| Image J | National Institute of Health | https://imagej.nih.gov |
| R-Package V.3.2.5 | The R project for Statistical computing | https://www.r- |
| | | project.org/ |
| Cell Ranger v 3.0.2 | 10x Genomics | https://support. 10xge |
| | | nomics.com/single- |
| | | cell-gene- |
| | | expression/software/ |
| Seurat v. 3.1.1 | Satija Lab | https://satijalab.org/s |
| | | eurat/ |
| EnrichR | Ma'ayan Lab | https://maayanlab.clo |
| | | ud/Enrichr/ |
| Experimental Model: Cell | | |
| lines | | |
| MM13 mouse embryonic stem | Tom Jessell Lab, Columbia University | |
| cell line | (Wichterle et al., 2002) | |
| Deposited data | | |
| Bulk and single-cell RNA-Seq | Gene Expression Omnibus (GEO) | GSE185891 |

### ESC Maintenance and Two-Dimensional (2D) Differentiation:

The mouse ESC line, MM13, was routinely maintained in ES cell media (DMEM+20% FBS) with 100u/ml of mouse leukocyte inducing factor (LIF). Cells were maintained on mitotically inactive irradiated mouse embryonic fibroblasts (MEFs) and were passaged at least twice before starting the differentiation. To prepare cells for the differentiation, ESC colonies were dissociated with 0.25% trypsin and plated on gelatin coated plates at 1:10 dilution to reduce MEF transfer. Cells cultured on gelatin coated plates and allowed to proliferate for 1-2 days. To initiate differentiation, cells were plated on 0.1% gelatin coated 24-well CellBIND dishes (Corning) at 20,000 cells/ml density in 0.5 ml/well N2/B27 medium that contains 10 ng/ml basic fibroblast growth factor (bFGF). The N2/B27 medium contains 1:1 portions of Advanced Dulbecco's modified Eagle Media (DMEM) F12 (Hyclone) and Neurobasal media (Thermo Fisher Scientific) supplemented with 0.5×N2, 1×B27 supplement (with Vitamin A), 2 mM L-glutamine, 0.1 mM 2-mercaptoethanol (ME), and 1% BSA. On day 1, small colonies of cells can be observed attached to the bottom of the wells. On day 2, cells were supplemented with 10 ng/ml bFGF and 5 μM CHIR99021 for 24 hours to induce a neuromesodermal identity (Gouti et al., 2014). On day 3, media was changed as follows: for protocol 1, cells were exposed to 100 nM RA for 48 hrs. For protocol 2, 10 ng/ml human recombinant BMP4 (Thermo Fisher Scientific) was added concomitantly with 100 nM RA (RA+BMP4) for 48 hrs. For protocol 3, cells were exposed to 100 nM RA for 24 hrs, followed by a pulse of RA+BMP4 for another 24 hours. To induce terminal differentiation in all three conditions, the medium containing growth factors was replaced with basic N2/B27 medium at day 5, and cell allowed to differentiate to day 9. At the end of the differentiation, the cultures were fixed for immunohistochemical analysis or lysed to obtain RNA for RNA-Seq or quantitative reverse transcriptase PCR analysis.

Bulk-RNA sequencing and data processing: Total cell lysate was obtained in buffer RLT (Qiagen) at different time points from three independent differentiations (biological replicates) conducted parallelly. RNA extraction was performed using RNeasy mini kit (Qiagen) and the quality was determined using Agilent Technologies 2100 Bioanalyzer and only samples with a RIN score >8.0 were sequenced. Stranded libraries were constructed using Universal plus mRNA sequencing kit (NuGEN) and sequenced onto 2 lanes of NovaseqS1 to generate 30 million reads/sample. Obtained reads were aligned to the latest mouse reference genome (mm10) using the STAR spliced read aligner. Pearson correlation and principal component analysis (PCA) was performed to determine the similarities among the samples and replicates. Differentially expressed genes were identified using DESeq and EdgeR package on R platform. The candidate genes were selected using cutoff false discovery rate (FDR <0.05) and log fold change (>2). Differentially expressed gene lists were further subjected to Enrichr, Metascape (Zhou et al., 2019) and DAVID functional annotation tools to identify enriched gene ontology (GO) categories.

WGCNA analysis: We used WGCNA package (Langfelder and Horvath, 2008) to identify the expression dynamics of gene modules associated with differentiations for protocol 1, 2, and 3. WGCNA was performed separately on RNA-Seq samples from protocol 1 (R-branch), 2 (C-branch), and 3 (B-branch) with day 0 and day 3 as common samples. First, differentially expressed genes were extracted for each group to determine the most variable genes. The Pearson correlation matrices were calculated for all RNA pairs in each group and were transformed into adjacency matrices using the power function. A dynamic tree-cut algorithm was used to identify gene co-expression modules where modules were defines as branches cut off from the tree and labeled in unique colors. The module eigengene (ME) represents the first principal component for each module. Gene ontologies associated with the modules were determined by supplying top 100 genes to the Enrichr platform.

Protein-protein interaction (PPI) network analysis: We used the metascape algorithm (www.metascape.org) to identify PPI networks induced by 6 hrs and 24 hrs of RA exposure. First, significant differentially expressed (DE) genes were extracted by pair-wise comparison with the day3 dataset (day3 v/s 6 hrs RA and day 3 v/s 24 hr RA) using log fold change>2 and false discovery rate (FDR)<0.01 as cut off for the upregulated genes. The entire gene list obtained from the DE analysis was then submitted to Metascape platform with Mus musculus as both input and analysis species. Both gene ontologies (GO) and PPI network were then retrieved for each gene list using the default Metascape parameters applied under the express analysis tool (minimum network size=3, maximum network size 500).

### Single-Cell RNA Seauencina and Analysis:

Preparation of single cell suspension: Day 9 cultures from both protocol1 (RA) and protocol3 (RA+BMP4) were dissociated with 0.25% cold trypsin for 5 mins. Trypsin activity was stopped with the addition of trypsin inhibitor (Sigma-Aldrich) in 1:1 ratio. Cells were then pelleted by centrifugation at 1000RPM and resuspended in 1× PBS. The ratio of viable cells was determined using Trypan blue staining. Dead cells were removed using MACS dead cell removal kit (Miltenyi Biotec Inc.) by following the manufacturer's protocol. Eluted live cells were then suspended in 1× PBS containing 0.04% BSA solution (400 μg/ml) for the library preparation.

Library preparation, and sequencing: ~10,000 live cells/conditions were used to construct single-cell specific cDNA libraries using protocol described in 10× Genomics chromium single cell 3′ reagent kit (v3.1 Chemistry). Briefly, cells were partitioned into nanoliter-scale Gel-Beads-in-emulsion (GEM) using the 10× Chromium controller. Each GEM contains a unique barcode which is shared among the cDNA generated from a single cell. Cells were then lysed, and cDNA synthesis and feature barcoding were performed in the GEMs. The sequencing libraries were recovered using Magnetic separation and the quantity and quality of cDNA were assessed by Agilent 2100 expert High Sensitivity DNA Assay. cDNA samples were sequenced on 1 lane of NovaSeq 6000 S2 flow cell and reads were mapped to mouse mm10 genome using Cell Ranger v.3.0.2 to generate fasq files.

Filtering genes and cells: Seurat package v3.1.1 was used for the analysis. For each condition, genes expressing in less than 3 cells were removed and cells with >250 features were retained. PCA was used for dimension reduction (npcs=50) with top 3000 most variable genes. Raw count data were normalized using regularized negative binomial regression with SCT transformation. Cell clustering is done using Shared Nearest Neighbor (SNN) Graph method and cluster specific markers were identified by Wilcox Rank Sum test.

Each cell cluster was annotated by a combination of the following methods 1) the expression of known canonical cell-type markers 2) Gene Set Enrichment Analysis (GSEA) of cluster specific markers determined by FindMarkers function in Seurat.

Reclustering of neuronal clusters: From the entire single-cell dataset, neuronal cells were first identified by the expression of pan-neuronal markers-Tubb3 and Elavl1/2. Roughly 30% cells express these markers in both protocol 1 and protocol 3 and were thus annotated as the mature neurons. The neuronal populations were then extracted using the Seurat package in R, by clustering the cells expressing Tubb3 >2 (median normalized log expression levels) using the Shared Nearest Neighbor (SNN) Graph method. This manipulation resulted in 12 clusters in protocol 1 (RA) and 13 clusters in protocol3 (RA+BMP4). Clusters were visualized with Uniform Manifold Approximation Projection (UMAP) method and the unique cluster-specific markers were identified using FindMarker function in Seurat.

GO analysis for neuronal clusters: We used DAVID functional annotation tool (Huang et al., 2009b) (https://david.ncifcrf.gov/tools.jsp) for identifying enriched biological processes and pathways associated with different neuronal clusters. For this, cluster-specific gene lists were sorted according to log fold change (FC) values and all genes with log FC=>0.2 were selected and submitted to the DAVID platform with default settings and Mus musculus as the input species. Ontological terms were then surveyed to determine if terms reflect the known functions of the dIs that the given cluster belongs to. Selected GO terms were arranged in the decreasing order of −Iog10 (p-value) and represented as bar plots.

Integration of single-cell datasets from different origins: The in vivo spinal cord single-cell dataset (Delile et at., 2019) was downloaded from ebi.ac.uk/arrayexpress/experiments/E-MTAB-7320/and loaded into R. Gene names were standardized to match our dataset using the biomaRt function (Durinck et al., 2009). This data was then loaded into Seurat (version 4.0) and processed using the SCTransform pipeline (Hafemeister and Satija, 2019). In vivo cell type labels were simplified and transferred to the in vitro datasets. The in vivo dataset was then integrated with the in vitro protocol datasets using Seurat's reciprocal PCA integration pipeline, and projected into a three-dimensional UMAP space. Tabula Muis reference datasets of kidney (2781 cells), lung (5449 cells) and trachea (11269 cells) were downloaded from figshare.com/articles/dataset/Robject_files_for_tissues_processed_by_Seurat/5821263, loaded into R, and then processed through Seurat, as above. The three Tabula Muis datasets were then integrated with the in vitro and in vivo spinal cord datasets using Seurat's reciprocal PCA integration pipeline and embedded into a three-dimensional UMAP space. Code for this analysis has been made available (See Gupta, S. et al., 2022, Cell Reports, Volume 40, Issue 3, Article number 111119.)

dI cell type extraction based on the marker gene expression: Cutoffs for gene expression levels when extracting cells were initially determined through the cluster-specific violin plots from the integrated in vivo and in vitro spinal cord datasets for the SCT assay. Cutoffs were then refined by plotting the extracted cells on a UMAP plot adjusting the values to exclude non-specific cells with background levels of the genes. The cutoffs values for each gene are as follows: dI1=BarhI1>1 (in vivo-417 cells, in vitro-1120 cells), dI2=Foxd3 >1 (in vivo-552 cells, in vitro-395 cells), dI3=IsI1>1 TIx3>0.5 (in vivo-1669 cells, in vitro-89 cells), dI4=Pax2>1 and Lhx1>1 (in vivo-1943 cells, in vitro-810 cells), dI5=Lmx1b >1 and Pou4f1>1 (in vivo-433 cells, in vitro-216 cells), dI6=Dmrt3>1 (in vivo-73 cells, in vitro-60 cells). To extract Tubb3+ cells in the in vivo and in vitro combined dataset, expression by cluster was plotted, and the clusters expressing high levels of Tubb3 were extracted as a whole. Code has been made available (See Gupta, S. et al., 2022, Cell Reports, Volume 40, Issue 3, Article number 111119.)

Embryoid body (EB) culture. and expansion: To initiate EB formation, dissociated embryonic stem cells were seeded in low attachment plates (Corning) at 10⁵ cells/ml density in N2/B27 medium supplemented with 10 ng/ml bFGF. To induce posterior spinal cord and dorsal spinal patterning, RA and RA+BMP4 were added using the same timeline as described for the 2D differentiation, and the media was changed at every other day after day 5.

For expansion cultures. EBs were dissociated in single cell suspension at day 6 by treating with 0.25% Trypsin, followed by trituration to break up the larger cell clumps. Cells were pelleted and passaged in N2/B27 medium containing either DMSO or 5 μM CHIR at 1:3 dilution. Media was changed at every third day during the expansion culture.

Embryoid body (EB) differentiation: To induce differentiation, EBs were dissociated into smaller clumps by a 5-7-minute treatment with cold 0.25% Trypsin (Gibco), followed by trituration. Trypsin was neutralized with 1:1 addition of Trypsin inhibitor (Sigma-Aldrich) and cells were pelleted by centrifugation at 1000RPM. Cell pellets were resuspended in N2/B27 medium (without growth factors) and plated onto a Matrigel precoated plate. In our experience, the single cell suspension of 100-150 EBs provided enough cells to seed one 24-well plate. Spontaneous neural differentiation is induced when dissociated EBs are cultured in N2/B27 medium for 10 days. At the end of 10 days, multiple neural processes can be visualized by phase contrast microscopy and cultures were either fixed with 4% PFA for immunohistochemistry or lysed to extract RNA in RLT buffer (Qiagen).

Immunohistochemistry: For 2d differentiations, adherent cultures were first washed with 1×PBS and fixed with fresh cold 4% PFA for 10 minutes in the well. Following fixation, cultures were washed twice with 1×PBS to remove any remaining PFA. Cells were blocked with 1×PBS with 1% heat inactivated horse serum for 1 hour and primary antibodies are added in the blocking solution for an overnight incubation at 4C. Following washes, species appropriate secondary antibodies (Jackson Immuno Research Laboratories) were added in 1× PBT (PBS+0.1% Triton 20) for 1 hr. The cultures were then washed with 1× PBT to remove traces of secondary antibodies and counterstained with DAPI to stain nuclei. Plates were then imaged on Zeiss LSM800 inverted confocal microscope at 10× magnification.

For EBs, EBs were collected and fixed with fresh 4% PFA for 20 minutes at 4° C. EBs were then equilibrated with 30% sucrose for overnight at 4C and embedded into the OCT compound for cryo-sectioning. 12 um sections were collected onto positive charged slides (VVVR), incubated with the antibody blocking solution for 1 hr, and then stained as above.

Sections were counterstained with DAPI, mounted in Prolong Gold and imaged using a Zeiss LSM800 inverted confocal microscope.

Quantitative (a) RT-PCR analysis: RNA was isolated with RNeasy Mini Kit (Qiagen, cat no. 74104), and converted to cDNA using Superscript IV First Strand Synthesis kit (Thermo Fisher Scientific). qRT-PCR was performed using SYBR Green PCR Master Mix (Applied Biosystems) on Roche 480 Lightcycler (Roche). The relative fold expression was calculated using 2-AACt method of comparing the expression of the target gene with that of the housekeeping gene glyceraldehyde 3-phosphate dehydrogenase (Gapdh). All experiments were repeated at least 2-3 times (technical replicates) from at least two independent differentiations (biological replicates), and data is represented as mean±SEM.

The primer sequences for all target genes are listed in the Table 5.

Image quantification: To count the number of nuclei, images were converted to 8 bit image in ImageJ, their threshold intensity was adjusted to capture only the florescent nuclei, and the number of nuclei counted using the analyze particle tool in ImageJ. For each image, the area under the DAPI staining was determined using ImageJ and target cells were represented as % of cells in 100 μm² DAPI⁺ area.

Statistics: Data are represented as meant SEM (standard error of the mean). Tests for statistical significance were performed using Prism software (version 9). Values of p<0.05 were considered significant in all cases.

### Results

Establishment of 2d-protocols to direct mESCs towards dorsal spinal interneuron fates.

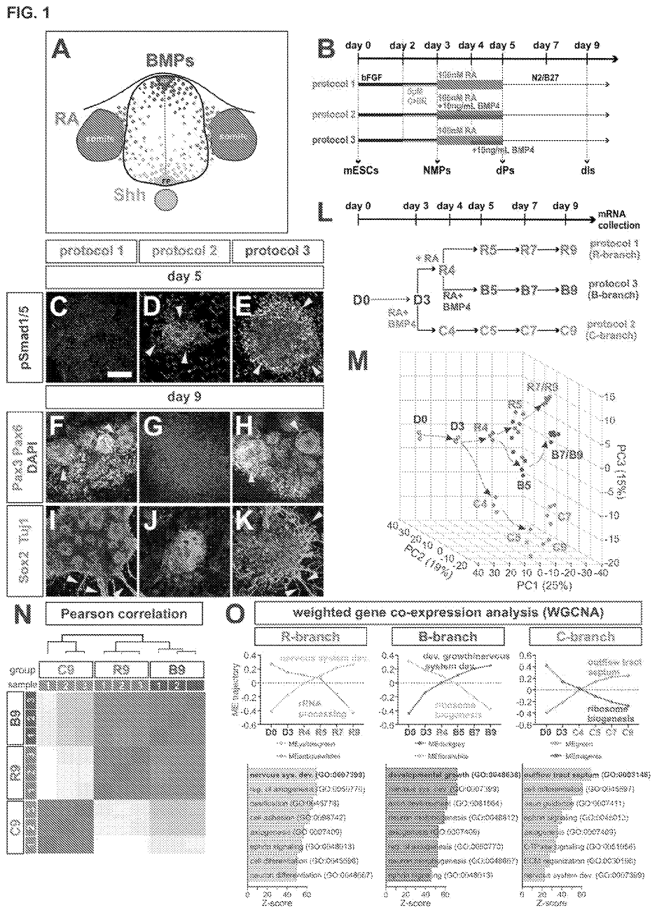

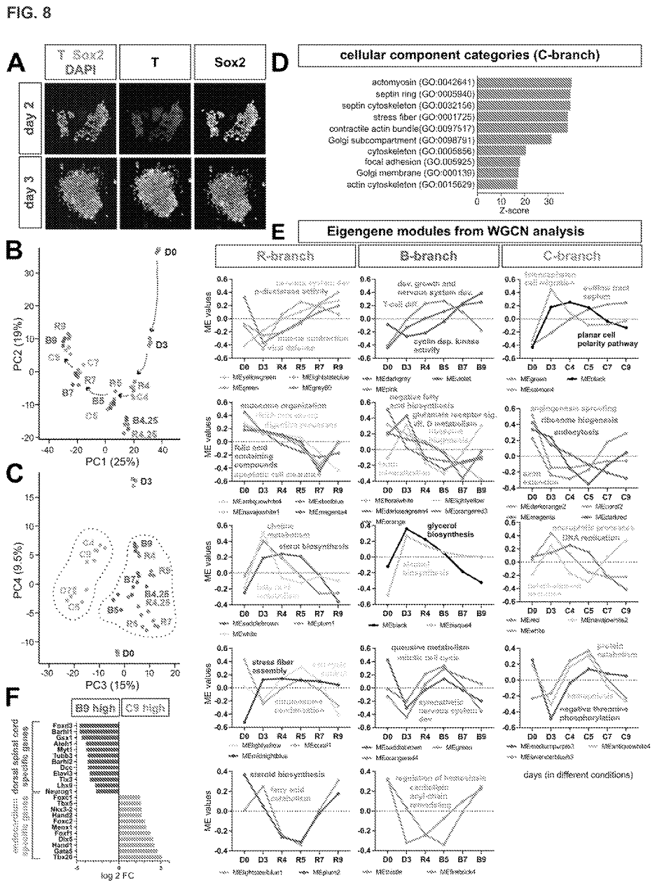

To direct dI fates through an NMP intermediate, we adapted a mESC directed differentiation protocol previously used to derive ventral spinal neurons (Gouti et al., 2014; Sagner et al., 2018). In this protocol, treatment with basic fibroblast growth factor (bFGF) and the Wnt agonist CHIR (FIG. 1B), results in ~90% of mESCs acquiring a bipotential NMP identity by day 3 in culture (FIG. 8A) distinguished by the co-expression of both mesodermal (Brachyury (T)) and neural progenitor cell (NPC, Sox2) markers (FIG. 8A).

Using the developmentally relevant growth factors (FIG. 1A) previously shown to direct ESCs towards dorsal spinal fates (Andrews et al., 2017; Duval et al., 2019; Gupta et al., 2018; Wilson et al., 2004), we then treated day 3 NMPs with 100 nM RA for 48 hours either alone (protocol 1, FIG. 1B) or in combination with 10 ng/ml BMP4 starting at either day 3 (protocol 2) or day 4 (protocol 3). Treatment with RA alone did not activate BMP signaling (distinguished by the absence of phosphorylated (p) Smad1/5/8 staining; FIG. 1C) and resulted in dorsalized neural cultures, with Pax3⁺ Pax6⁺ Sox2⁺ neural rosettes extending Tuj1⁺ neurites (arrowheads, FIGS. 1F, 1I). When BMP4 was added in combination with RA, BMP signaling was now activated (arrowheads, FIGS. 1D, E).

However, the timing of BMP4 addition critically affected NMP identity. When BMP4 was added concomitantly with RA at day 3, we did not observe the generation of Pax3⁺ (FIG. 1G) or Sox2⁺ (FIG. 1J) cells. The aggregates exhibited Tuj1 staining, but had no apparent neuronal morphology (FIG. 1J). In contrast, when RA was added at day 3, followed by BMP4 at day 4, we again observed Pax3⁺ Pax6⁺ Sox2⁺ neural rosettes, which robustly extended Tuj1⁺ neurites (arrowheads, FIGS. 1H, 1K).

Unbiased transcriptomic analyses identify the developmental trajectories leading to either spinal cord or cardiac fates.

To assess the transcriptional changes that resulted from the timed addition of RA±BMP4, we conducted bulk RNA-Seq analyses at different timepoints along the timelines of the three protocols (FIG. 1L). Principal component (PC) analyses (PCA) (Son et al., 2018) revealed that PC1 and PC2 contributes 44% of variation in the data, and separates the least (D0, D3) from the most (D7, D9) differentiated samples (FIG. 8B), while PC3 contributes 15% variance and separates the samples collected from different protocols (FIG. 8C). Combining PC1, PC2 and PC3 into a three-dimensional PCA plot reveals three differentiation trajectories (FIG. 1M), connected by bifurcation points at day 3 and day 4. The C-branch (RA+BMP4, protocol 2) follows a distinct trajectory from the R-branch (RA, protocol 1) after bifurcating at day 3, while the B-branch (RA+BMP4, protocol 3) bifurcates from the R-branch at day 4 (FIG. 1M). The R- and B-branches follow similar trajectories and terminate in adjacent PCA spaces, distinct from the C-branch, suggesting that their differentiation program is largely equivalent. Supporting this conclusion, comparing their global transcription profile using a Pearson correlation demonstrated that the most similarity was between the R- and B-branches, which were divergent from the C-branch (FIG. 1N).

We next examined whether the three protocols direct distinct differentiation outcomes, by assessing which groups of genes are most upregulated over time using a weighted gene co-expression and network analysis (WGCNA) (Langfelder and Horvath, 2008). Distinct eigengene modules (Farhadian et al., 2021; Panahi et al., 2020), i.e., a collection of genes that exhibit similar expression trends, were identified for each branch (summarized in FIG. 8E). Of these, the yellow-green, dark-grey and green modules show increasing expression from day 0 to 9 for protocols 1, 3, and 2 respectively (FIG. 10). The genes in these modules were then subjected to a functional enrichment analysis using Enrichr (Chen et al., 2013). Both the yellow-green and dark-grey gene modules were highly enriched for gene ontology (GO) terms related to nervous system development and neural differentiation (z-score>30) (FIG. 1O), supporting the hypothesis that protocols 1 and 3 promote neural identities. In contrast, the top GO term in the green module is “outflow tract septum” (FIG. 1O), a structure that partitions the outflow tracts in the heart to regulate the blood flow (Webb et al., 2003). Other cardiac-related terms, such as actomyosin, contractile actin filament, and stress fibers terms are represented in the cellular component categories (FIG. 8D). Supporting the conclusion that protocol 2 is promoting cardiac fates, multiple cardiac-specific genes are upregulated by day 9 in cells derived from protocol 2 (FIG. 8F).

Taken together, these analyses reveal the timeline over which RA and BMPs act sequentially to direct NMPs towards the neural lineages, rather than mesodermal fates.

ScRNA-Seq of mESC-derived neurons identifies the full complement of spinal dorsal interneurons.

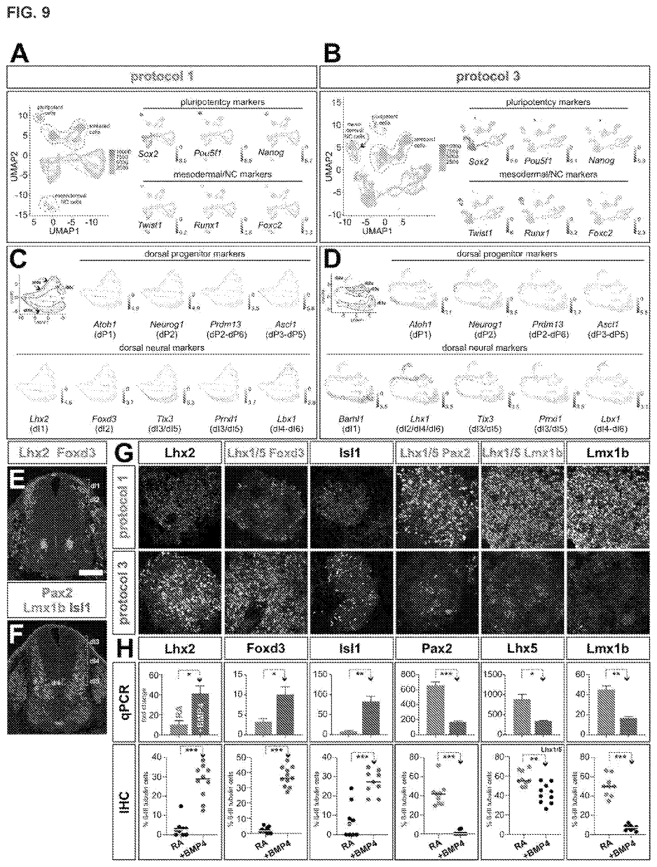

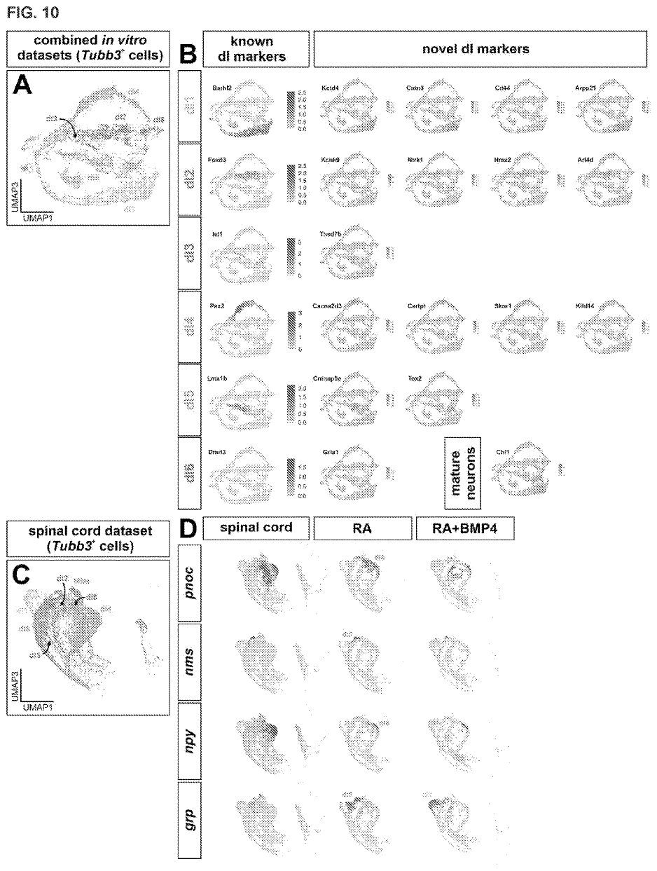

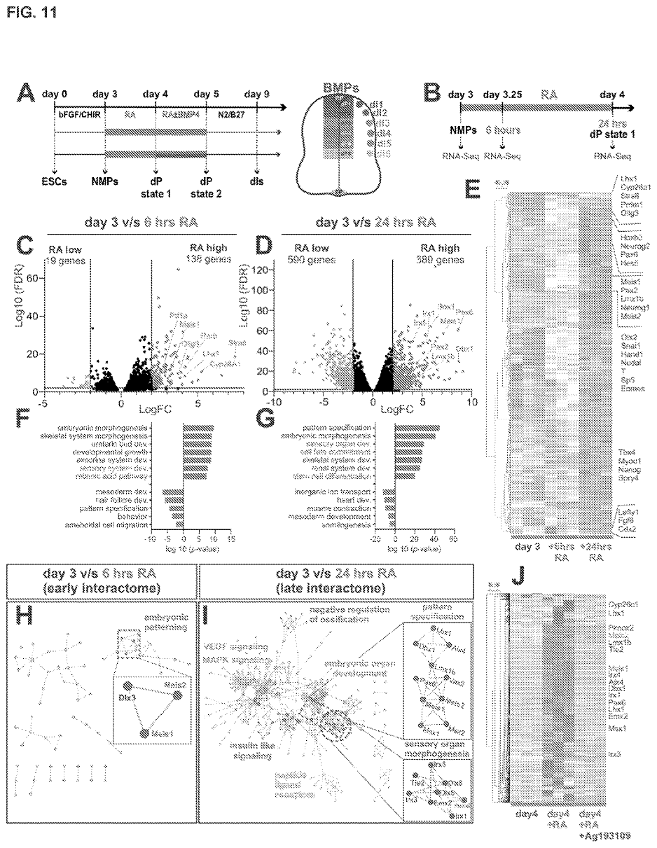

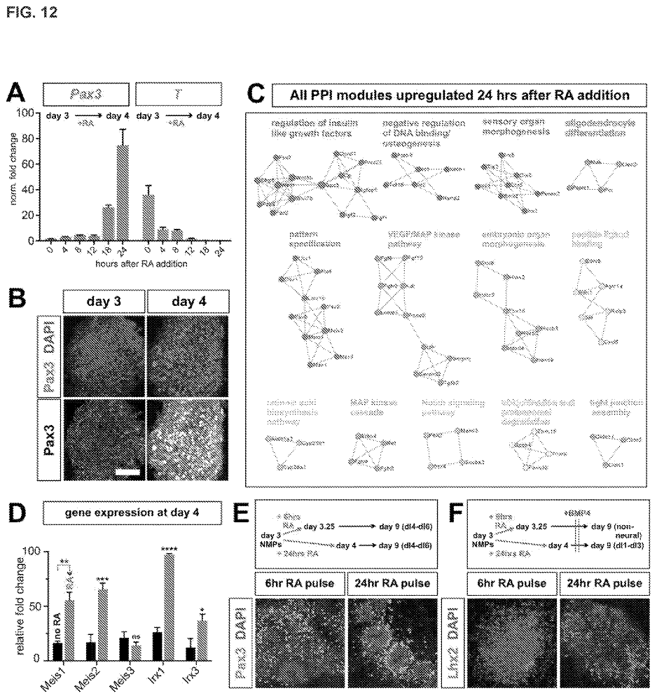

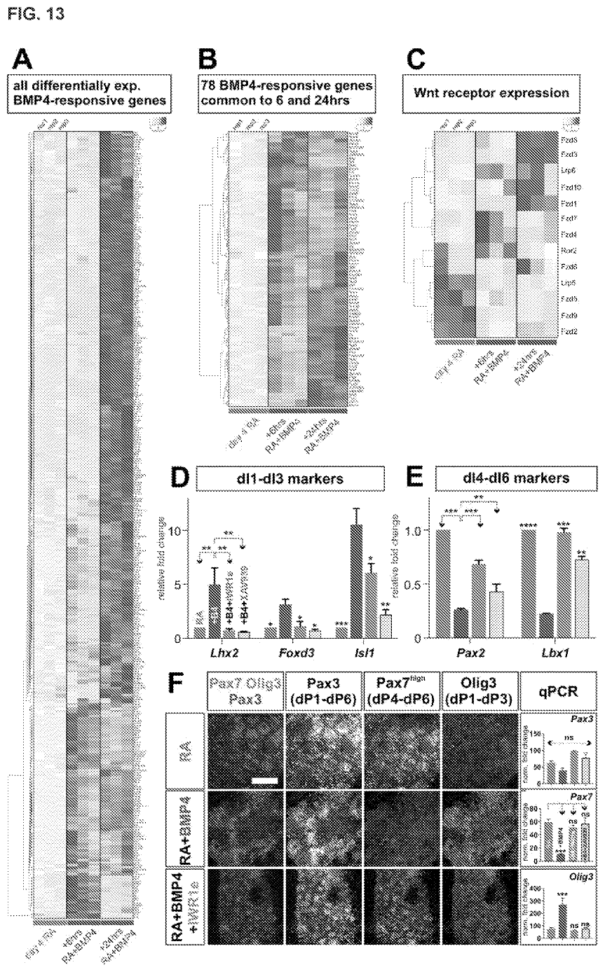

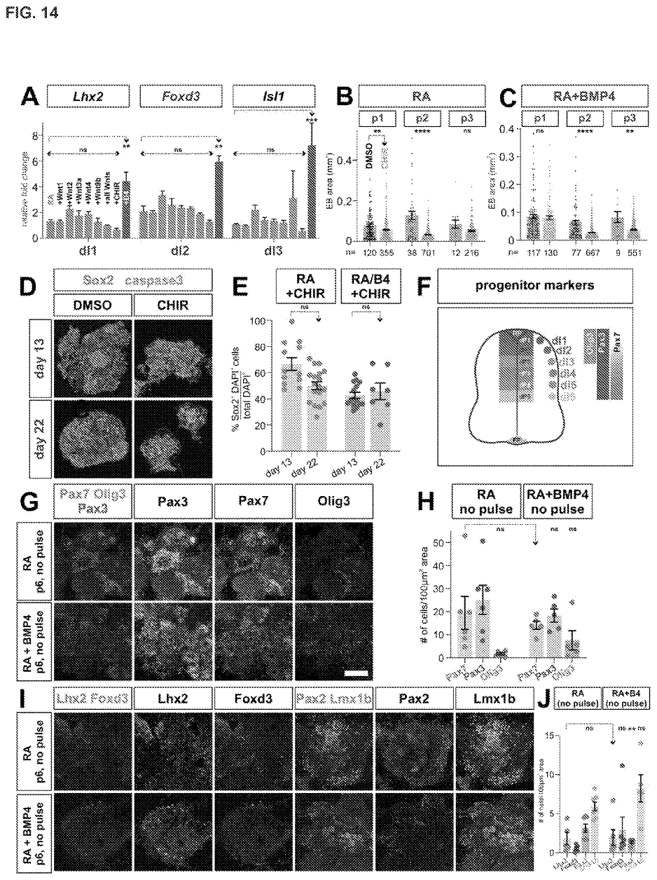

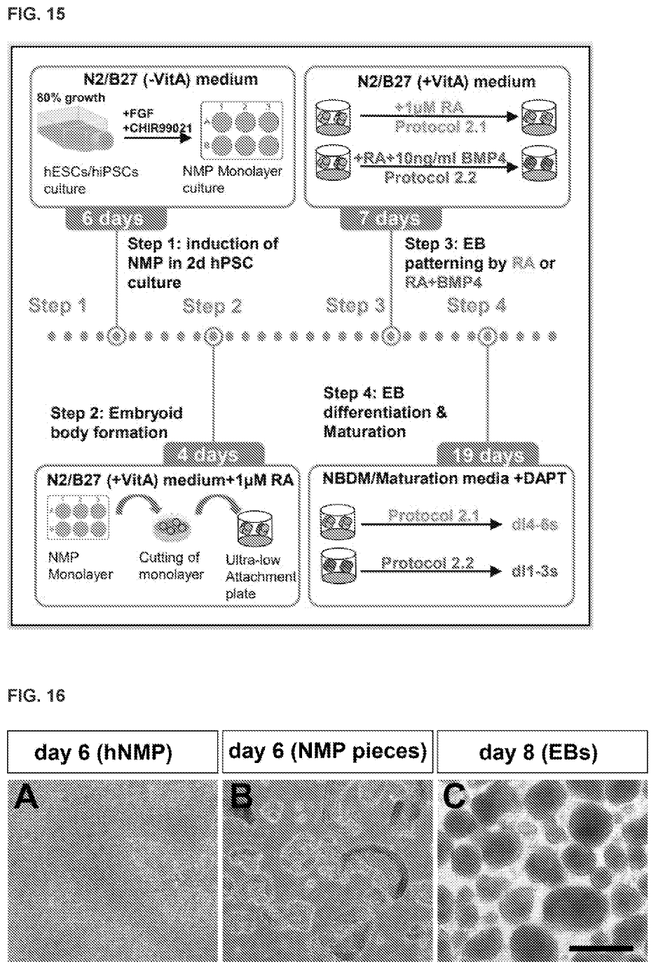

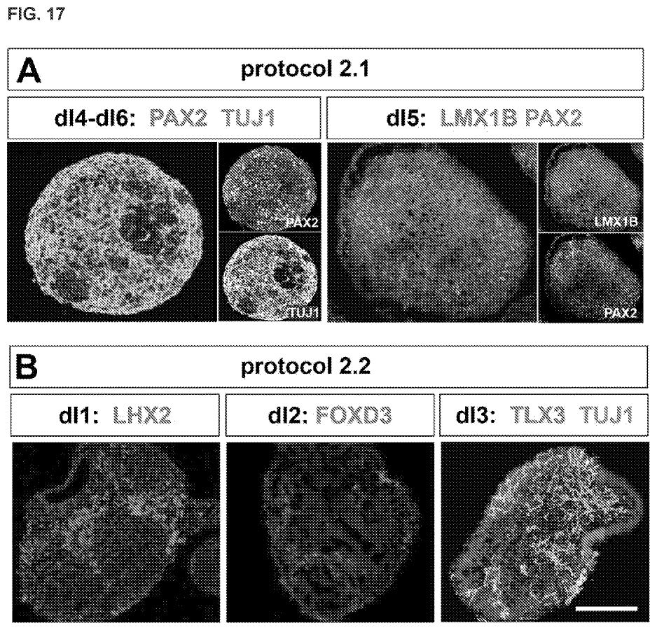

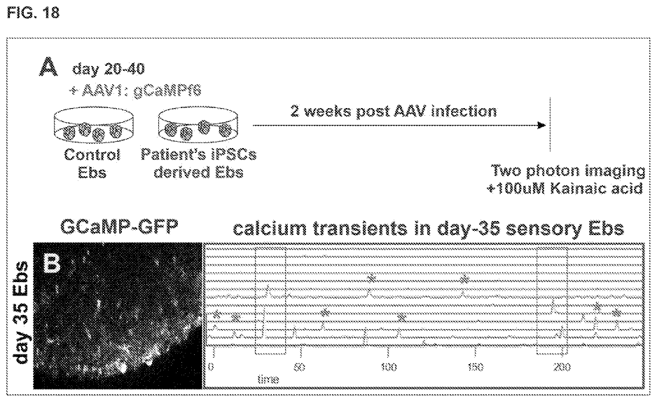

To assess whether protocols 1 and 3 direct distinct classes of dIs, we obtained single-cell transcriptomes from 9,704 cells using protocol 1 and 13,121 cells using protocol 3, taken at day 9 of the differentiation procedure (FIG. 2A). We performed Louvain clustering followed by Uniform Manifold Approximation and Projection (UMAP) plots, to visualize groups of transcriptionally distinct cell types (Becht et al., 2018). This pipeline identified 21 dusters of cells in both protocol 1 (FIG. 2C) and protocol 3 (FIG. 2D). In both conditions, ~30% of cells identify as being stressed; they cluster separately and display <2000 RNA counts (FIGS. 2C, 2D, 9A, 9B). ~2-4% cells are pluripotent stem cells, marked by Sox2, Pou5f1 (Oct4) and Nanog expression, while 5-8% cells are non-neural, either of mesodermal or cardiac neural crest identity, expressing Twist1, Runx2, and Hand2 (Han et al., 2021; Soldatov et al., 2019; Vincentz et al., 2013) (FIGS. 2C, 2D, 9A, 9B). The remaining ~60% cells have a neural identity, either Sox2⁺ NPCs or Tubb3⁺ (class Ill s-tubulin, Tuj1) differentiated neurons (feature plots, FIGS. 2C, 2D).

To distinguish the neuronal subtypes, we subclustered the Tubb3 cells, resulting in 14 (protocol 1) and 13 (protocol 2) transcriptionally distinct clusters (FIGS. 2E, 2F). We then used a well-characterized panel of transcription factors (FIG. 2B) to assign dI identities to these clusters (Gupta and Butler, 2021; Lai et al., 2016). This analysis revealed that protocol 1 primarily generates the dI4, dI5 and dI6s that mediate pain, itch, and heat perception, while protocol 3 most notably generates the dI1, dI2 and dI3s that regulate proprioception, gait, and mechanosensation. Specifically, in protocol 1 the majority of subclustered Tubb3⁺ cells divide into two major groups of closely aligned clusters (FIGS. 2B, 2E, 9C): one group (clusters 7, 2, 0, and 1) expresses Lmx1b, Prrxl1, and TIx1/3 which define dI5 identity (Lai et al., 2016). The second group (clusters 6, 12, 13, 4, 5, 3) co-express Pax2 and Lhx1 which denotes dI4/dI6 identity. Of these clusters, only cluster 6 also expresses Dmrt3, an established marker of dI6s (Andersson et al., 2012). The neurotransmitter profile supports these fate designations: phenocopying endogenous neurons (FIG. 2B) the dI4/dI6 clusters express Gad2, a GABA synthesizing enzyme (Pillai et al., 2007), while the dI5 clusters express SLC17a6 (vGlut2), a glutamate transporter specific to excitatory neurons (FIG. 2E) (Cheng et al., 2004). Of the remaining four clusters, cluster 8 and 9 map to dI1 and dI2 identities (FIG. 9C), while cluster 10 is enriched for neural and neuroendocrine specific genes, and cluster 11 is enriched for ribosomal genes (Table 1).

In protocol 3, the majority of subclustered Tubb3 cells divide into three groups of closely aligned clusters (FIG. 2B, 2F, 9D): clusters 7, 1, 0 and 8 express Lhx2/9 and Banh/12, which denote dI1 identity, clusters 11, 5 and 6 express the dI2 markers Foxd3, Lhx1 and Lhx5, while cluster 12, and 10 express Is/1 and TIx3, which designate them as dI3s. As in vivo, all three classes of in vitro-derived neurons are excitatory, expressing either SIc17a6 or Grn2b (FIG. 2F). Of the remaining four clusters, clusters 2 and 9 express Atoh1 and Neurog1 respectively, which mark the dP1 and dP2 state, and suggests these cells are immature dI1 and dI2s (FIG. 9D). Cluster 4 expresses dI4-specific markers (FIG. 9D) and cluster 3 express multiple ribosomal and mitochondrial genes indicative of dying neurons (Ilicic et al., 2016) (Table 1).

In summary, protocol 1 (RA) generates ~33% dI4s, 40.5% dI5s, 8% dI6s, 5% dI1s, 5% dI2s, as well as 8.5% unknown neural cell types (pie chart, FIG. 2E). In contrast, protocol 3 (RA+BMP4) generates ~45.5% dI1s, 29% dI2s, 8% dI3s, 8% dI4s, as well as 9.5% unknown cell types (pie chart, FIG. 2F). Both immunohistochemical and qRT-PCR analyses support the findings obtained from the scRNA-seq data (FIG. 9E-9H).

### GO Analyses of ESC-Derived dIs Identifies Sensory Modality-Specific Ontology Modules

We next assessed whether the functional identities of these in vitro derived neurons could be predicted from the transcriptomic data. In vivo, the dIs relay diverse somatosensory modalities either locally within the spinal cord (Fernandes et al., 2016; Todd, 2017), or by long-range afferent connections (Wercberger and Basbaum, 2019). Generally, the most dorsal populations regulate proprioception (dI1/dI2) (Sakai et al., 2012; Yuengert et al., 2015) and touch-induced mechanosensation (dI3s) (Bui et al., 2013) while the more intermediate classes form sensory relay circuits for pain, itch, heat, and touch (dI4/dI5) (Koch et al., 2018; Todd, 2010). The dI6s regulate coordinated movement by forming inhibitory synapses with spinal motor neurons, while themselves receiving cholinergic and glutamatergic inputs (Perry et al., 2019).

To identify functionally relevant gene ontologies, we selected the cluster for each dI population that most specifically expressed the appropriate differentiation markers (>0.2 log fold change), and subjected them to the DAVID pipeline (Huang et al., 2009a; b), which identifies enriched biological processes and signaling pathways related to diseases or drugs (FIG. 2G). Remarkably, each class of ESC-derived dIs displayed the relevant modality-specific signatures. For the dorsal-most neurons, we analyzed cluster 0 (dI1), cluster 5 (dI2), and cluster 10 (dI3) and specifically found enriched GO categories related to balance and walking behaviors, as well as excitatory synaptic signaling. Both dI1 and dI2 clusters additionally include proprioception-related terms (FIG. 2G, Table 2). For the more intermediate neurons, we analyzed cluster 4 (dI4), cluster 1 (dI5), and cluster 6 (dI6) The dI4/dI5 clusters include GO terms associated with pain and itch perception, specifically implicating both oxytocin, a neuropeptide that modulates pain processing (Boll et al., 2018) and -alanine, an amino acid that induces itch (Liu et al., 2012). Also mirroring their endogenous functionality, the dI4 cluster contains terms related to inhibitory synaptic signaling, while the d1S cluster is enriched for excitatory synaptic terms (FIG. 2G, Table 2). In contrast, the dI6 cluster contains multiple terms associated with coordinated movement, and synaptic signaling pathways, accurately reflecting their functional identity. Finally, we also found enriched terms in the dI clusters related to different drug addiction pathways, including amphetamine and endocannabinoids for dI1/dI2s, and amphetamine, morphine and cocaine for dI4/dI5s (FIG. 2G, 10A, Table 2). Such signatures suggest the dIs as novel cellular targets for the ability of psychoactive drugs to modulate pain and itch perception (Lipman and Yosipovitch, 2021) or proprioception (Downey et al., 2017). Other novel dI-subtype markers identified in this analysis include receptors, ion channels and adhesion molecules (FIG. 1OA, 10B; Table 4).

ESC-Derived dIs are Transcriptionally Indistinguishable from Endogenous Spinal Interneurons

To assess whether the ESC-derived dIs are also transcriptionally similar to their in vivo counterparts, we used reciprocal PCA based data integration and label transfer (Hao et al., 2021) to compare a single-cell dataset taken from embryonic stage (E) 9.5-E13.5 mouse spinal cords (Delile et al., 2019) with the in vitro-derived cell types in protocols 1 and 3.

Projecting the combined in vivo and in vitro datasets into UMAP plots revealed a remarkable degree of overlap (FIG. 3A, 3B, 3C). This overlap was only observed for the neural progenitor and neural identities; the blood, mesoderm, neural crest and skin lineages present in the in vivo dataset were notably absent from the in vitro datasets (FIG. 3A).

While cells from protocol 1 and 3 mapped directly on top of the spinal cord cells (FIG. 3B), these three datasets were distinct from trachea, lung, and kidney cells (Consortium et al., 2018) (FIG. 3E).

To compare the distribution of cell types in the in vivo and in vitro datasets, we performed unsupervised clustering on an integrated dataset, which yielded a total of 29 clusters (FIG. 3C); 24 clusters (0-10,12-25) were common to all datasets, while 6 clusters were unique to the spinal cord dataset. Using a dot plot analysis to assess the distribution of dI subtype markers within these 24 clusters, we found striking similarity between the in vivo and in vitro-derived datasets (FIG. 3D). While there are modest differences, the overall concordance between both the number of cells expressing a given dI marker, and the strength of marker expression, suggests a high degree of similarity between the in vivo and in vitro cell clusters. This concordance continues as the neurons mature, demonstrated by the expression of neuropeptides, which modulate the intensity of sensory inputs in the adult spinal cord (Lai et al., 2016; Russ et al., 2021). Specific neuropeptides, including prepronociceptin (pnoc; dI2, dI4), neuromedin S (nms; dI4) neuropeptide Y (npy, dI4), gastrin releasing peptide (grp; dI1, dI5), are enriched in the same populations of dIs in both the in vitro and in vivo datasets (FIG. 1OC, 10D). Thus, the ESC-derived dis appear to proceed along the same maturation process as endogenous spinal neurons.

Finally, to assess whether the in vitro-derived dI populations specifically mirror their endogenous counterparts, we compared the protocol 1 vs. protocol 3 datasets to an atlas of the neuronal classes of the spinal cord (FIG. 3F), generated using the annotation provided in (Delile et al., 2019). The in vivo dI1-dI3s, and dI4-dI6s, map to identical positions in protocol 3 (RA+BMP4) and protocol 1 (RA) respectively (FIG. 3F). In contrast, there was no overlap between the ventral spinal neurons, such as the motor neurons (MNs), with the in vitro datasets (FIG. 3F). We also computationally extracted the cells expressing specific dI subtype markers from the in vitro and in vivo datasets and projected them in the same UMAP space (FIG. 3G). This analysis further demonstrated that all six classes of in vitro-derived dIs are transcriptionally indistinguishable from their spinal cord-derived counterparts.

Taken together, these studies suggest that our in vitro protocols are accurately replicating the in vivo developmental program of the spinal cord permitting the generation of bona ide sensory interneurons.

RA Activates a Transcriptional Network that Specifies a dI4-dI6 Default State

We took advantage of the directed differentiation protocols to investigate the unresolved mechanisms by which RA and BMP direct dI fate specification. To further validate the use of a stem cell model, we first assessed the genetic basis of the fate decisions mediated by RA signaling. During the 24 hr pulse of RA at day 3, Pax3 levels gradually increase, while T expression concomitantly declines (FIG. 12A). By day 4, ~90-95% of cells are Pax3′ (FIG. 12B), suggesting that the NMPs transition to a dorsal neural progenitor state (dP state 1) during this time period (FIG. 11A). To identify the transcriptional changes occurring during this first commitment step, we performed bulk RNA-Seq at day 3 (NMP state), day 3.25 (early transcriptional response to RA) and day 4 (dP state1) (FIG. 11B).

Using differential expression analyses, we identified 138 genes significantly upregulated after 6 hrs of RA treatment while 19 genes were significantly downregulated (FIG. 11C). Upregulated genes include previously identified RA-regulated factors, such as Meis1, Meis2, Stra8, Cyp26a1, and Rarb, confirming that the cells are directly responding to RA. By 24 hours of RA exposure, 389 genes were significantly upregulated while 590 genes were significantly downregulated (FIG. 11D). The upregulated genes include genes present in intermediate dPs and the dI4/dI5s, while the downregulated genes include mesodermal specific genes (FIG. 11E-11G). These findings support the hypothesis that RA induces NMPs towards intermediate spinal identities, while also directing them away from mesodermal fates, thereby ensuring the transition from an NMP to Pax3+dP state 1.

We next assessed whether the dP state1 can be defined as a function-specific interaction network, by subjecting the upregulated genes to a Metascape protein-protein interaction (PPI) analysis (Zhou et al., 2019). At 6 hrs, only a sparsely connected, nascent PPI network exists, with one functional module for embryonic patterning containing the RA-target genes Meis1 and Meis2 (FIG. 11H, inset). However, by 24 hrs, the PPI network has expanded into 13 functional modules (FIGS. 11I, 12C). Four modules, insulin-like signaling, pattern specification, sensory organ morphogenesis, and peptide ligand receptor, contained genes expressed in the intermediate spinal cord (insets, FIGS. 11I, 12C). Notably, Meis1 and Meis2 interact with Pax6, Lmx1b, Msx1 and Msx2, in the pattern specification module.

Using both qRT-PCR (FIG. 12D) and transcriptional profiling of untreated day 4 cells (control), or after a 24-hour pulse of RA±AGN193109, a pan-RAR inhibitor (FIG. 11J), we independently validated that Meis1 and Meis2, but not Meis3, are upregulated by RA, and that many patterning genes are RA responsive. Thus, Meis1/2 are good candidates for the key transcription factors that link RA signaling to the initiation of spinal cord patterning, i.e. dP state1.

### Wnt Signaling is Induced as an Immediate Consequence of BMP4 Treatment

We hypothesized that the addition of BMP4 to NPCs in dP state1, directs them to a more dorsal dP state, i.e. dP state2 (FIG. 11A). To define the transcriptional changes occurring after BMP4 addition, we performed bulk RNA-Seq at day 4 (dP state1), day 4.25 (early transcriptional response to RA+BMP4) and at day 5 (dP state2) (FIG. 4A). We identified that 106 genes were upregulated by 6 hrs of BMP4 addition, while 305 genes were upregulated after 24 hrs of BMP4 exposure (FIG. 13A). 78 genes, out of 411, were common between both time points (FIGS. 4B, 13B). Focusing on these common genes, we identified enriched functional processes using GO analyses. The GO terms included the BMP signaling pathway (FIG. 4C, Table 3), confirming that the analysis identified BMP responsive genes.

Additionally, we also found enriched GO terms related to Wnt signaling across three functional categories (FIG. 4C). Both Wnt ligands, including Wnt1, Wnt2, Wnt3, Wnt3a and Wnt4, and Wnt receptors, frizzled (Fzd) 8, Fzd3, Fzd10 and Lrp6 (FIG. 12C) were upregulated in response to BMP4 treatment along with Wnt signaling regulators, such as Dkk, Lmx1a (Hoekstra et al., 2013) and Bambi (Zhao et al., 2020). (FIGS. 4D, 4E, 12B). Selected Wnt-signaling genes were further assessed in a qPCR analysis of day 5 NMPs to validate whether they are BMP4-responsive. We found that expression of all genes tested significantly increased after the addition of BMP4, compared to the RA alone condition (FIG. 4F). The upregulation of Wnt expression was suppressed if Noggin, a BMP inhibitor, was concomitantly added with BMP4 on day 4 (FIG. 4F).

Many of the upregulated Wnts, Wnt1, Wnt3a, and Wnt4, are expressed in the developing dorsal spinal cord in vivo (Agalliu et al., 2009; Daneman et al., 2009) and signal through the canonical Wnt signaling pathway (MacDonald et al., 2009), suggesting that canonical Wnt signaling mediates the ability of BMP4 to direct the dP state1 to dP state2 transition.

### Inhibiting Canonical Wnt Signaling Blocks the Induction of the Dorsal-Most Fates

To assess the role of canonical Wnt signaling in dI fate specification, we added endo-IWR1 (IWR1e), a small molecule inhibitor which blocks p-catenin function (Chen et al., 2009) to dPs from day 4-6 (FIG. 5A). By day 9, the RA+BMP4 condition robustly induces dlis-dI3s (FIG. 5F-51, 5N-5P). In contrast, the addition of IWR1e resulted in the loss of dI1s and dI2s (FIG. 5J, 5K, 5N, 50) while dI3 differentiation was less affected (FIG. 5L, 5P).

BMP4 directs the dorsal-most fates (dI1-dI3), while concomitantly suppressing intermediate dorsal fates (dI4-dI6, FIGS. 51, 13D-13E), which are RA-dependent (FIG. 5E). Supporting a role for the Wnts mediating the latter activity, there is no suppression of Pax2+dI4/dI6 and Lmx1b+dI5 fates when IWR1e is added to the dP cultures, along with RA+BMP4 (Figure SM, SQ). The addition of IWR1e did not erode Pax3+pan-dorsal identity or Pax7 expression, but nevertheless did suppress Olig3 (FIG. 12F), which may contribute to the loss of dI1/2 and perdurance of dI4-6 fates.

Together, these results demonstrate that the BMP4-dependent dI fates require canonical Wnt/p-catenin signaling. In its absence, BMP4-mediated suppression is lost, and dPs retain their intermediate spinal cord identity.

### Wnt Signaling Promotes BMP4-Dependent Fates by Regulating Proliferation, not Patterning

Previous studies have suggested that Wnts act ether as mitogens (Dickinson et al., 1994; Megason and McMahon, 2002) or patterning factors (Alvarez-Medina et al., 2008; Muroyama et al., 2002) in the specification of the dorsal spinal cord. We assessed these models, first by determining the effect of canonical Wnts (Wnt1, Wnt2, and Wnt3a), a non-canonical Wnt (Wnt9b) and a small molecule Wnt agonist (CHIR99021) on dI identity in the RA±BMP4 protocols. When Wnts were added in combination with RA, they were unable to induce the dorsal-most dIs by day 9 (FIG. 14A). Similarly, when the Wnts were added concomitantly with BMP4, there was no significant effect on the specification of dorsal spinal identity (FIG. 6A, 6B). Thus, Wnts are not sufficient to direct dI fate identity.

We then asked whether activating Wnt signaling in mESC-derived NMPs affected their mitotic index. To better visualize proliferation, mESCs were permitted to assemble into three dimensional EBs (Andrews et al., 2017; Duval et al., 2019; Gupta et al., 2018), and then treated with RA±BMP4 over the same timeline as protocol 1 and 3 (FIG. 1B) to induce dorsal spinal cord patterning. EBs were then cultured with either DMSO or CHIR from day 5 to day 9 (FIG. 6C), when the cultures were pulsed with 5-Ethynyl-2′-deoxyuridine (EdU) to label cells in S-phase of the cell cycle. Strikingly, there was a ~3-fold increase in EdU+cells in CHIR-treated EBs (FIG. 6E, 6G, 6H) compared to DMSO control (FIG. 6D, 6F, 6H) in both protocols. Similarly, the CHIR-treated EBs had a ~2-4-fold increase in the number of pHistone H3 (pH3)⁺ cells in M-phase, compared to control (FIG. 6D-6G,6I). Many of the dividing cells in the CHIR-treated cultures were Sox2+NPCs (FIG. 6E, 6G), while NPCs are largely depleted by day 9 in control cultures (FIG. 6D, 6F). Thus, extending the window of Wnt signaling appears to prolong NPC proliferation. These CHIR-treated NPCs retained their original identity, i.e., NPCs from protocol 1 expressed Pax3 and high levels of Pax7, while NPCs from protocol 3 expressed Pax3 and Olig3 (FIG. 6J-6P).

Together, these studies support the hypothesis that Wnt/P-catenin signaling functions as a mitogen for dorsal spinal NPCs, rather than specifying distinct dI identities.

Wnt-catenin signaling can be modulated to extend the timeline of dP patterning.

Finally, we assessed whether the mitogenic properties of Wnt signaling can be leveraged to expand the pool of mESC-derived NPCs. RA±BMP4 patterned EBs were serially passaged at 1:3 dilution every 7 days in the presence of DMSO (control) or CHIR (FIG. 7A). While the number of EBs were comparable in the DMSO and CHIR conditions on the first passage, they declined sharply by the second passage in the controls (FIGS. 7B, 7C, 7E, 7F, 7G, 7I, 14B, 14C). By the third passage, few EBs were observed in the DMSO condition, while the number of CHIR-treated EBs had increased by ~3- to 6-fold (FIG. 7E, 7I, 14B, 14C). The ability of CHIR to expand the number of EBs was observed in both RA±BMP4 conditions, but was most robust for the RA+BMP4 patterned EBs (FIG. 7E, 7I). CHIR-treated EBs remained Sox2⁺ after multiple passages, suggesting that they maintained their NPC identity (FIG. 7D, 7H, 14D, 14E).

To assess whether the expanded pool of NPCs retain the capacity to differentiate, we dissociated RA±BMP4 CHIR-treated EBs at passages 6 (FIG. 7J) or 7 onto Matrigel-coated plates, and cultured the resulting cells in the presence of CHIR, DMSO (i.e. CHIR withdrawal), or DAPT, a pro-differentiation agent (Crawford and Roelink, 2007) (FIG. 7J). By day 10, the CHIR-treated cultures continued to be solely Sox2⁺ (FIG. 7K, 7N, 7Q,) while DMSO-treated cultures had dramatically increased the expression of post-mitotic neural markers, neuronal nuclei (NeuN/Rbfox3), and Tubb3 (FIG. 7Q) and contained many Tuj1⁺ neurites (FIG. 7L, 70). Thus, the removal of CHIR triggers spontaneous neural differentiation. The addition of DAPT did not significantly augment NeuN and Tubb3 expression compared to DMSO (FIG. 7Q) although there was increased sprouting of Tuj⁺ neurites (FIG. 7M, 7P).

We next asked whether specific dI fates are preserved in the expanded NPC pool.

By day 10 (timeline in FIG. 7J), passage 6/7 cultures derived from RA-patterned EBs contained many Lmx1b⁺ dI5s, but only a few Pax2⁺ dI4/dI6s, while cultures from the RA+BMP4-patterned EBs contained both IsI1+dI3s and Lhx1b dI5s, but almost no Lhx2+dI1s, or Foxd3+dI2s (FIGS. 141, 14J, 7R, 7T, 7U). Thus, while some patterning information is preserved over many passages, prolonged CHIR treatment erodes its fidelity, with RA+BMP4 dPs now expressing high levels of Pax7 and low levels of Olig3, as if they have been returned to an intermediate spinal identity (FIG. 14G, 14H). However, dorsal patterning information can be restored, by including a pulse of RA±BMP4 between day 3 and 5 during the differentiation of the expanded EBs on Matrigel (FIG. 7J). The addition of RA resulted in a ~6-fold increase in the number of dI4/dI6s (FIG. 7S, 7X), while the addition of RA+BMP4 induced ~6-fold more dI1s, and ~2-fold dI2s (FIG. 7V, 7X), compared to control (FIG. 7R, 7T, 7X). Unexpectedly, the pulse of RA+BMP4 suppressed the dI3 fate (FIG. 7U, 7W, 7X). This strategy was successful at restoring almost all dI identities in cultures taken from EBs both at the earliest passage and at the latest passage assessed (passage 7;

FIG. 7X).

Thus, these data show that the chemical activation of the canonical Wnt signaling can be successfully used to dramatically expand the size of mESC-derived NPC population, and thereby to derive large numbers of specific dI populations.

### Discussion

The development of stem cell-based directed differentiation protocols results in both a source of in vitro-derived cell-types and a model system in which to identify novel developmental mechanisms. We have built on previous studies that derive spinal motor neurons in vitro from an NMP intermediate (Gouti et al., 2014; Sagner et al., 2018), to develop protocols that produce a complete in vitro atlas of dorsal spinal sensory neurons.

Our transcriptomic analyses suggest that these neurons are indistinguishable, both molecularly and according to their predicted functions from their endogenous counterparts.

We then used these protocols to investigate the mechanisms that regulate dI fate choices. These studies identified the hierarchy by which dI identities are first specified and resolved the role of Wnt signaling as a mitogen in this process. We then leveraged this mechanistic understanding to expand the numbers of dPs as a means of increasing dI yield.

### Hierarchy of dI Fate Specification

Previous studies have shown that NMPs contribute to spinal cord and paraxial mesodermal lineages both in vivo (Attardi et al., 2018; Tzouanacou et al., 2009) and in vitro (Gouti et aL., 2014). Our transcriptomic analyses revealed that the dI differentiation program conforms to the canonical Waddington model (Rajagopal and Stanger, 2016; Waddington, 1957) where NMPs are at the top of a “hill” of possible fates, and the time and sequence in which they receive RA and BMP4 signals controlling their differentiation into dIs vs mesodermal fates. Thus, the addition of RA at day 3 channels NMPs towards the intermediate dI fates. However, if BMP4 is added concomitantly with RA, the differentiation trajectory alters to specify endocardial lineages. BMP4 can only induce the dorsal-most dI fates after 24 hours of RA treatment, suggesting that NMPs must move through an RA-induced early competency state (dP state1), before BMP4 can relay dorsal spinal patterning information. These studies thereby inadvertently developed a directed differentiation protocol (protocol 2) that is a starting point for the specification of cardiogenic mesoderm, critical cell types needed to regenerate the heart (Duelen and Sampaolesi, 2017). BMP4 may first act to induce NMPs to form lateral plate mesoderm (Row et al., 2018), which contributes multiple cell types to the embryonic heart. This finding also suggests that NMPs may have more pluripotency for mesodermal fates than previously realized. In this case, the use of alternative growth factors in protocol 2, will direct NMPs towards additional mesodermal derivatives.

Our studies suggest that RA is first required to establish dP state 1, before specifying the dI4-dI6 fates. The Meisl12 genes, which are dynamically expressed in the early spinal cord neuroepithelium (SAnchez-Guardado et al., 2011), may be central to this competency state. Meis1/2 are among the earliest genes to be induced by RA (Gouti et al., 2017; Oulad-Abdelghani et al., 1997), and become central to a gene interaction network that contains the genes that specify intermediate spinal identity. Thus, the upregulation of Meis1/2 may drive dP state1, a multipotential progenitor state that differentiates into the dI4, dI5 and dI6s, after reiterative exposure to RA. The addition of BMP4 in turn, both suppresses dI4-dI6 fates, and induces another multipotent state, dP state 2, that differentiates into dI1, dI2 and dI3. Interestingly, dP state1 may be a default state, which was first suggested by earlier studies in the intermediate spinal cord (Diez del Corral et al., 2003: Novitch et al., 2003). When progenitors initially programmed with RA+BMP4 were taken through the expansion culture protocol, they appear to revert to the dP state1 identity. This reversion can in turn be reversed: a pulse of RA+BMP4 remains sufficient to convert dP state1 back to dP state2 and elicit formation of the dorsal most dis.

Extent to which In Vitro-Derived Dis Phenocopy Endogenous Sensory Interneurons

Our studies significantly extend previous studies seeking to generate dIs (Andrews et al., 2017; Duval et al., 2019; Gupta et al., 2018) by directly assessing the heterogeneity of the cultures, mapping their developmental trajectories, and documenting the extent to which stem cell-derived dIs resemble their endogenous counterparts. Using scRNA-Seq analyses, it was possible both to resolve which cell types are generated and make a more complete assessment of their molecular signatures. We thereby identified that our protocols generate the full complement of dorsal sensory interneurons, that there is no heterogeneity beyond dorsal spinal derivatives in the Tubb3+ cells, and that the transcriptomes of in vitro- and in vivo-derived dIs appear to be indistinguishable. In vitro-derived dIs express the correct patterning factors and neurotransmitters, and display the relevant functional GO terms, suggesting they encode the correct sensory modalities. For example, balance-related GO terms were enriched for dI1, dI2, dI3 and dI6, which regulate proprioception and gait, while pain and itch GO terms were enriched for dI4 and dI5, which relay noxious stimuli in vivo.

Moreover, we also find GO terms related to serotonergic and dopaminergic synapses, which may permit communication between the spinal cord and enteric nervous system. This signature is associated with the synapses formed between spinal neurons in the dorsal horn and the descending afferents from the brain stem (Schwaller et al., 2017) and diencephalon (Skagerberg and Lindvall, 1985), which together regulate both pain perception (Gautier et al., 2017) and sensation in gut (Travagli et al., 2006) and bladder (Hou et al., 2021). These circuits are of considerable therapeutic importance, given that damage to spinal circuitry in SCI patients can lead to both irritable bowel syndrome (Holmes and Blanke, 2019) and loss of bladder control (Hou et al., 2021; Taweel and Seyam, 2015). Additionally, we identified gene signatures related to addictive psychoactive compounds like morphine, cocaine, and amphetamine, suggesting the dIs are the cellular targets for these drugs, elevating their importance for drug screening platforms.

While this study is the first comprehensive analysis showing that directed differential protocols can generate bona fide, sensory modality-specific spinal interneurons, some hurdles remain. In each protocol, a subset of dIs arise as heterogenous populations, and the yield of dI3 and dI6, the populations that regulate mechanosensation and gait, remains modest. Further transcriptomic analyses are needed to assess the mechanisms by which dP state1 resolves to dI4-dI6, and dP state2 resolves into dI1-dI3. The identification of any dI-specific regulators might permit the generation of large numbers of a specific dI population using the CHIR-mediated expansion protocol. Finally, in vitro derived dIs need to be electrophysiologically assessed to determine their maturity; such an analysis requires an understanding of the electrophysiological signatures of endogenous spinal sensory neurons, which is not yet well defined.

### Role and Application of Wnt Signaling as a Mitogen

The BMPs have reiterative roles regulating dI fate specification and modulating the cell-cycle (Andrews et at., 2017; Ille et al., 2007). Here, we demonstrate that the control of proliferation is mediated through the activation of Wnt signaling. Like the BMPs, multiple Wnt genes, especially those that activate the canonical Wnt/beta-catenin pathway, are expressed in the developing dorsal spinal cord (Agalliu et at., 2009). Interactions between the BMP and Wnt pathways have been observed in other contexts, including during neural crest delamination (Burstyn-Cohen et al., 2004) and the assignment of hematopoietic fates (Lengerke et al., 2008). However, the role of the Whits in the spinal cord has remained unresolved. Previous studies manipulating the activity of Wnt signaling in vivo ascribed both dI fate specification (Muroyama et at., 2002; Zechner et al., 2007) and mitogenic roles (Ille et at., 2007; Megason and McMahon, 2002) to the dorsal Wnts. Our data unambiguously suggest that the Wnts tested so far are not sufficient to pattern dI fates, rather they function as mitogens in this context, perhaps controlling the size of the dorsal-most dI populations. It remains unresolved as to why there are so many Wnts present in the spinal cord in vivo.

One possibility is that they have signal-specific activities, like the BMPs (Andrews et at., 2017), sculpting the number of cells in each dI population. This mechanistic insight enabled us to expand mESC-derived spinal cultures by adding CHIR to constitutively activate Wnt/p-catenin signaling after the patterning stages. Remarkably this manipulation sustained dorsal spinal neural identity while enabling the dP to proliferate for more than 8 passages (the maximum number we attempted).

This protocol modification may represent a key step toward the long-sought goal of being able to supply specific neural populations in a limitless manner. Previous studies using neurospheres have suggested that immortalized cells lose their neurogenic potential over time (Olivos-Cisneros et al., 2021; Shen et al., 2006). We rather find, in this expansion protocol, that even the progenitors from the latest passages we tried (passage 5-7), retain the ability to differentiate as neurons. However, while neurogenesis remains remarkably stable, the patterning information does erode over time. Cell cycle duration has been shown to regulate cell fate by acting as a filter for long transcripts (Abou Chakra et al., 2021; Singh et al., 2013). Such mechanisms may affect the maintenance of cellular identity (i.e. dP state 2) when NPCs are held in an extended proliferative phase. As mentioned previously, the complement of dI fates can be again restored with a 2-day pulse of RA±BMP4.

### Implications of Stem Cell-Derived Sensory Neurons for Drug Screening and Regenerative Therapies

These studies have identified that distinct psychoactive and analgesic drug-related signaling pathways are present in different dI populations. This finding raises the possibility of using stem cell derived dis in drug screening studies to identify novel analgesics, and to study the mechanistic action of drugs such as cocaine and amphetamine at the cellular level.

Targeting a spinal cellular substrate for therapeutics, that concomitantly spare cortical activation, may result in more specific pain relief, that avoids the addictive side effects that have led to opioid abuse.

One can assess the regenerative potential of these cells by determining the extent of functional recovery after they are transplanted into an injured or diseased spinal cord. At present, it remains unresolved whether the transplantation of pure populations of neurons (Fortin et al., 2016), will be more functionally beneficial than replacing populations of multipotential progenitors or mixtures of neurons (Kawai et al., 2021). The identification of new dI-specific cell surface markers in this study (Table 4) enables the sorting of specific neural populations, which will make it possible to distinguish between these possibilities.

In summary, these studies uncover new mechanistic insights into the process of dI differentiation in vitro, which are then applied to derive bona fide spinal sensory neurons in the large numbers needed for clinical and drug discovery applications. They represent a critical step towards using ESC-derived dIs for studying the effects of various addictive compounds and in vitro disease modeling for pain disorders.

### REFERENCES

Abou Chakra, M., et al. (2021). et al. Elife 10. 10.7554/eLife.64951.
Agalliu, D., et al. (2009). Neuron 61, 708-720. 10.1016fj.neuron.2008.12.026.
Alvarez-Medina, R., et al. (2008). Development 135, 237-247.
Andersson, L. S., et al. (2012). Nature 488, 642-646. 10.1038/nature11399.
Andrews, M. G., et al. (2017). Elife 6. 10.7554/eLife.30647.
Andrews, M. G., et al. (2019). Curr Top Dev Biol 132, 417-450.
Antonic, A., et al. (2013). PLoS Biol 11, e1001738.
Attardi, A., et al. (2018). Development 145. 10.1242/dev.166728.
Becht, E., et al. (2018). Nat Biotechnol. 10.1038/nbt.4314.
Boll, S., et al. et al. (2018). Neuroscience 387, 149-161.
Bui, T. V., et al. (2013). Neuron 78, 191-204.
Burstyn-Cohen, T., et al. (2004). CDevelopment 131, 5327-5339.
Chen, B., et al. (2009). Nat Chem Biol 5, 100-107.
Chen, E. Y., et al. (2013). BMC Bioinformatics 14, 128.
Cheng, L., et al. (2004). Nat Neurosci 7, 510-517.
Consortium, T. M., et al. (2018). Nature 562, 367-372.
Crawford, T. Q., and Roelink, H. (2007). Dev Dyn 236, 886-892.
Daneman, R., et al. (2009). Proc Nati Acad Sci USA 106, 641-646.
Delile, J., et al. (2019). Development 146.
Dickinson, M. E., et al. (1994). Development 120, 1453-1471.
Diez del Corral, R., et al. (2003). Neuron 40, 65-79.
Downey, L. A., et al. (2017). J Clin Pharmacol 57, 1330-1337.
Duelen, R., and Sampaolesi, M. (2017). EBioMedicine 16, 30-40.
Durinck, S., et al. (2009). Nat Protoc 4, 1184-1191.
Duval, N., et al. (2019). Development 146.
Faravelli, I., et al. (2014). Stem Cell Res Ther 5, 87.
Farhadian, M., et al. (2021). Sci Rep 11, 2367.
Fernandes, E. C., et al. (2016). Pain 157, 475-487.
Fortin, J. M., et al. (2016). Sci Rep 6, 23579.
Furusawa, K., et al. (2011). Spinal Cord 49, 49-54.
Gautier, A., et al. (2017). Exp Neurol 288, 11-24.
Gouti, M., et al. (2017). Dev Cell 41, 243-261.e247.
Gouti, M., et al. (2014). PLoS Biol 12, e1001937.
Gupta, S., and Butler, S. J. (2021). WIREs Mech Dis. 13, e1520.
Gupta, S., et al. (2018). Stem cell reports 10, 390-405.
Gupta, S., et al. (2021). STAR Protoc 2, 100319.
Hafemeister, C., and Satija, R. (2019). Genome Biol 20, 296.
Haimson, B., et al. 2020). bioRxiv, 2020.2001.2007.898072.
Han, X., et al. (2021). Elife 10.
Hao, Y., et al. (2021). Cell 184, 3573-3587.e3529.
Hazen, V. M., et al. (2012). Dev Biol 367, 216-227.
Hoekstra, E. J., et al. (2013). PLoS One 8, e74049.
Holmes, G. M., and Blanke, E. N. (2019). Exp Neurol 320, 113009.
Hou, S., DeFinis, et al. (2021). eNeuro 8.
Huang, d. W., et al. (2009a). Nucleic Acids Res 37, 1-13.
Huang, d. W., et al. (2009b). Nat Protoc 4, 44-57.
Ilicic, T., et al. (2016). Genome Biol 17, 29.
Ille, F., et al. (2007). Dev Biol 304, 394-408.
Ionta, S., et al. (2016). Sci Rep 6, 20144.
Kattman, S. J., et al. (2011). Cell Stem Cell 8, 228-240.
Kawaguchi, J., et al. (2005). Bone 36, 758-769.
Kawai, M., et al. (2021). Cell Rep 37, 110019.
Koch, S .C., et al. (2018). Annu Rev Physiol 80, 189-217.
Kong, D., et al. (2021). Stem Cell Res Ther 12, 172.
Ladd, A. N., et al. (1998). Dev Biol 204, 407-419.
Lai, H. C., et al. (2011). The Journal of Neuroscience 31, 10859-10871.
Lai, H. C., et al. (2016). Development 143, 3434-3448.
Langfelder, P., and Horvath, S. (2008). BMC Bioinformatics 9, 559.
Le Dreau, G., et al. (2012). Development 139, 259-268.
Lengerke, C., et al. (2008). Cell Stem Cell 2, 72-82.
Lenggenhager, B., et al. (2012). PLoS One 7, e50757.
Lipman, Z. M., and Yosipovitch, G. (2021). J Am Acad Dermatol 84,148-155.
Liu, Q., et al. (2012). The Journal of Neuroscience 32, 14532-14537.
MacDonald, B.T., et al. (2009). Dev Cell 17, 9-26.
Megason, S. G., and McMahon, A.P. (2002). Development 129, 2087-2098.
Muller, T., et al. (2005). Genes Dev 19, 733-743.
Muroyama, Y., et al. (2002). Genes Dev 16, 548-553.
Novitch, B. G., et al. (2003). Neuron 40, 81-95.
Olivos-Cisneros, L., et al. (2021). IBRO Neurosci Rep 10, 75-82.
Oulad-Abdelghani, M., et al. (1997). Dev Dyn 210, 173-183.
Panahi, B., et al. (2020). PLoS One 15, e0225677.
Perry, S., et al. (2019). The Journal of Neuroscience 39, 1771-1782.
Pillai, A., et al. (2007). Development 134, 357-366. 10.1242/dev.02717.
Rajagopal, J., and Stanger, B. Z. (2016). Dev Cell 36, 133-137.
Rodrigo Albors, A., et al. (2018). Development (Cambridge, England) 145.
Row, R. H., et al. (2018). Elife 7.
Russ, D. E., et al. (2021). Nat Commun 12, 5722.
Sagner, A., et al. (2018). PLoS Biol 16, e2003127.
Sakai, N., et al. (2012). The Journal of Neuroscience 32, 15377-15387.
Sinchez-Guardado, L., et al. (2011). Dev Dyn 240, 1475-1492.
Schwaller, F., et al. (2017). Sci Rep 7, 2215.
Shen, Q., et al. (2006). Nat Neurosci 9, 743-751.
Singh, A. M., et al. (2013). Stem cell reports 1, 532-544.
Skagerberg, G., and Lindvall, O. (1985). Brain Res 342, 340-351.
Soldatov, R., et al. (2019). Science 364.
Son, K., et al. (2018). Biomed Res Int 2018, 2906292.
Taweel, W. A., and Seyam, R. (2015). Res Rep Urol 7, 85-99.
Todd, A. J. (2010). Nat Rev Neurosci 11, 823-836.
Todd, A. J. (2017). Mol Pain 13, 1744806917693003.
Travagli, R. A., et al. (2006). Annu Rev Physiol 68, 279-305.
Trawczynski, M., et al. (2019). Front Cell Neurosci 13, 369.
Tzouanacou, E., et al. (2009). Dev Cell 17, 365-376.
Vincentz, J. W., et al. (2013). PLoS Genet 9, e1003405.
Waddington, C. H. (1957). The strategy of the genes.
Watanabe-Susaki, K., et al. (2014). Stem Cells 32, 3099-3111.
Webb, S., et al. (2003). J Anat 202, 327-342.
Wercberger, R., and Basbaum, A. I. (2019). Curr Opin Physiol 11, 109-115.
Wchterle, H., L et al. (2002). Cell 110, 385-397.
Wlson, L., et al. (2004). Dev Biol 269, 433-446.
Woolnough, J. L., et al. (2016). PLoS One 11, e0157276.
Yuengert, R., et al. (2015). Cell Rep 13, 1258-1271.
Zechner, D., et al. (2007). Dev Biol 303, 181-190.
Zhao, H. J., et al. (2020). Cell Signal 67, 109489.
Zhou, Y., et al. (2019). Nat Commun 10, 1523.

TABLE 1
Top 10 genes for each dl cluster identified in the Tubb3+ cell types.
Protocol 1 Tubb3 clusters:
dl4 clusters

| Cluster3 | Cluster5 | Cluster4 | Cluster12 | Cluster13 |
|---|---|---|---|---|
| Vim | Pax2 | Cartpt | Cartpt | Sst |
| Pax3 | Robo3 | Runx1t1 | Sst | Samd5 |
| Btg2 | Sst | Lhx1 | Slco3a1 | Pnoc |
| Cdkn1c | Nhln2 | Gad2 | Spock3 | Irx2 |
| Gadd45g | Lhx1 | Ptprd | Mab21l2 | Cdh8 |
| Neurod4 | Cntn2 | Zic1 | Kcnq3 | Gm17750 |
| Hes6 | Igsf8 | Cntn1 | Lhx1 | Vcan |
| Ccnd1 | Pid1 | Lhx1os | Zdbf2 | Nova1 |
| Btbd17 | Mxra7 | Pax2 | Dlgap1 | Cacna2d3 |
| Fabp7 | Gad2 | Bcl11a | Zfhx3 | Gpm6a |
| Tmsb4x | Lhx5 | Plcb4 | Fam135b | Meg3 |
| Neurog1 | Racgap1 | Pax8 | Sema3a | Bend4 |
| Mdk | Hoxb5os | Skor1 | Phlda1 | Phlda1 |
| Msx3 | Ppp1r17 | Arid5b | Apbb2 | Pax8 |

dl5 cluster

| Cluster7 | Cluster2 | Cluster0 | Cluster1 | |
|---|---|---|---|---|
| Fabp7 | Tlx3 | Gm2694 | Pou3f1 | |
| Gadd45g | Ebf2 | Thsd7a | Reln | |
| Ptn | Crabp1 | Tshz1 | C130073E24Rik | |
| Ckb | Cdkn1c | Cacna2d1 | Tshz2 | |
| Hes6 | Tcf12 | Fstl5 | Chl1 | |
| Vim | Racgap1 | Crmp1 | Nrxn1 | |
| Msx3 | Tlx1 | Tmem163 | Pbx3 | |
| Dll1 | Prrxl1 | Rgmb | Scn3b | |
| Ccnd1 | Tcf4 | Nr2f1 | Nell2 | |
| Ascl1 | Lmx1b | Igfbpl1 | Zfhx3 | |
| Btg2 | Spire1 | Cbln1 | Chmp2b | |
| Smoc1 | Dll3 | Cntnap5a | Mapt | |
| Ezr | Myo1b | Pdzrn4 | Zfhx4 | |
| Pax3 | Trp53i11 | Cntn2 | Slc17a6 | |
| dl6 cluster | dl1 cluster | dl2 cluster | Unidentified clusters | |
| Cluster6 | Cluster8 | Cluster9 | Cluster10 | Cluster11 |
| D930028M14Rik | Crabp1 | Akap12 | Nefl | AY036118 |
| Snhg11 | Rassf4 | Nefm | Gap43 | Hmgb2 |
| Gap43 | Epha5 | Nefl | Syt4 | Rps29 |
| Nrxn3 | Barhl1 | Gm2694 | Nefm | Rps26 |
| Myt1l | Igfbp5 | Ntm | Sncg | Ppia |
| Nrp2 | Atoh1 | Enc1 | Pou2f2 | Tuba1b |
| Olfm1 | Gm28050 | Gm12688 | Pcsk1n | Top2a |
| Pou2f2 | 1810058l24Rik | Cadps | Nrxn1 | Gm42418 |
| Meg3 | Barhl2 | Rapgef6 | Uchl1 | Rpl37 |
| Onecut2 | Cnr1 | Cdk14 | Stmn4 | Txn1 |
| Snrpn | Pou3f1 | Hmx2 | Tubb2a | Tmsb10 |
| Mapt | Tmem163 | Foxd3 | Hoxc8 | H2afz |
| Nova1 | Lhx2 | Jun | Nr2f2 | Rpl39 |
| Syt4 | Pou3f2 | Cdkn1a | Rapgef6 | Fau |

Protocol 3 Tubb3 clusters:
dI1 clusters

| Cluster7 | Cluster1 | Cluster0 | Cluster8 |
|---|---|---|---|
| Ebf1 | Meis2 | Chmp2b | Zcchc12 |
| Epha5 | Ank2 | Robo2 | Gap43 |
| Lhx2 | Robo3 | Rprm | Snhg11 |
| Grem2 | Rgmb | Chl1 | Sorcs3 |
| Podxl2 | Nrcam | Gm45159 | Pou2f2 |
| Lhx9 | Barhl2 | Cntn3 | Lhx9 |
| Rassf4 | Dnm3 | Barhl1 | Arhgap20 |
| Rnd2 | Grin2a | Nav3 | Mab21l2 |
| Insm1 | Pdzrn4 | Cttnbp2 | Zdbf2 |
| Atp2b2 | Pou3f1 | Sorcs3 | Tshz2 |
| Cntn2 | Gm28050 | Reln | Ebf3 |
| Lnpk | Bcl11a | Meg3 | Gm45159 |
| 1700025G04Rik | Slc17a6 | Gpm6a | Nrxn1 |
| Necab2 | Fam171b | Crabp1 | Meis1 |

dI2 clusters

| Cluster11 | Cluster5 | Cluster6 |
|---|---|---|
| Nhlh2 | Gm2694 | Nr4a2 |
| Nkain4 | Akap12 | Foxp2 |
| Cdkn1c | Robo3 | Pik3r1 |
| Igsf8 | Lhx1 | Ntm |
| Sst | Cdk14 | Jun |
| Cacna2d1 | Nefm | Epha5 |
| Rnd2 | Foxd3 | Foxd3 |
| Tubb3 | Cbln1 | Flrt3 |
| Nhlh1 | Enc1 | Ddit3 |
| Tbc1d9 | Ntm | Stmn4 |
| Pou4f1 | Arl4d | Gm12688 |
| Cntn2 | Hmx2 | Egr1 |
| Myo1b | Nefl | Herpud1 |
| Ppp1r17 | Tpbg | Pnoc |

dI3 clusters

| Cluster12 | Cluster10 |
|---|---|
| Tlx3 | Sncg |
| Gm2694 | D930028M14Rik |
| Fstl5 | Gap43 |
| Nr2f1 | Pou2f2 |
| Pbx3 | Snhg11 |
| Ebf1 | Syt4 |
| Pou4f1 | Nefl |
| Cacna2d1 | Pcsk1n |
| Stk39 | Rprm |
| Thsd7a | Sst |
| C130073E24Rik | Stmn2 |
| Lmx1b | Mapt |
| Ube2ql1 | Cartpt |
| Tubb2a | Onecut2 |

dI4 cluster
Cluster4
Cartpt
Sst
Pax2
Ptprd
Zic1
Gad2
Lhx1
Slc32a1
Nrxn3
Pax8
Pid1
Dner
Auts2
Rnd3

dP1/dP2 clusters

| Cluster9 | Cluster2 |
|---|---|
| Igfbp5 | Cdkn1c |
| Gadd45g | Gadd45g |
| 1810058I24Rik | Vim |
| Rassf4 | Btg2 |
| Atoh1 | Neurog2 |
| Hes6 | Neurod4 |
| Nkd1 | Pax3 |
| Sox2 | Hes6 |
| Ly6h | Btbd17 |
| Vim | Tcf4 |
| Prkar2a | Fabp7 |
| Wls | Tfap2b |
| Rbp1 | Neurog1 |
| Sparc | Nhlh1 |

unidentified
Cluster3
AY036118
Fabp7
Dbi
Col14a1
Igfbp5
Gm42418
Rps26
Mdk
mt-Nd2
Rps29
Id3
Hmgb2
Fth1
Rpl41

TABLE 2
GO categories for each dl cluster.
Protocol 3 Cluster 0 Pathway

| All terms | PValue | -log10 (p value) |
|---|---|---|
| mmu04360: Axon guidance* | 2.65E−04 | 3.577333 |
| mmu04728: Dopaminergic synapse* | 0.002377 | 2.624022 |
| mmu04724: Glutamatergic synapse* | 0.008444 | 2.073465 |
| mmu04514: Cell adhesion molecules (CAMs) | 0.026541 | 1.576081 |
| mmu04723: Retrograde endocannabinoid signaling* | 0.035362 | 1.451459 |
| mmu04721: Synaptic vesicle cycle* | 0.070046 | 1.154615 |
| mmu04550: Signaling pathways regulating pluripotency of stem | 0.072348 | 1.140576 |
| cells | | |
| mmu04010: MAPK signaling pathway | 0.098787 | 1.0053 |

| Selected terms | -log10 (p value) |
|---|---|
| mmu04360: Axon guidance | 3.577332661 |
| mmu04728: Dopaminergic synapse | 2.624021748 |
| mmu04724: Glutamatergic synapse | 2.073464864 |
| mmu04723: Retrograde endocannabinoid signaling | 1.451459333 |
| mmu04721: Synaptic vesicle cycle | 1.15461505 |

Protocol 3 Cluster 5 BioProcess

| All terms | PValue | -log10 (p value) |
|---|---|---|
| GO: 0007399~nervous system development* | 1.06E−12 | 11.9733 |
| GO: 0007275~multicellular organism development | 8.82E−07 | 6.054768 |
| GO: 0006351~transcription, DNA-templated | 1.35E−06 | 5.870001 |
| GO: 0007411~axon guidance | 2.14E−06 | 5.670091 |
| GO: 0006355~regulation of transcription, DNA-templated | 3.25E−05 | 4.488345 |
| GO: 0010977~negative regulation of neuron projection | 1.28E−04 | 3.893174 |
| development | | |
| GO: 0048485~sympathetic nervous system development* | 2.73E−04 | 3.563093 |
| GO: 0000122~negative regulation of transcription from RNA | 4.57E−04 | 3.339681 |
| polymerase II promoter | | |
| GO: 0031133~regulation of axon diameter | 8.50E−04 | 3.070504 |
| GO: 0030154~cell differentiation | 9.15E−04 | 3.038639 |
| GO: 0008045~motor neuron axon guidance | 0.001239 | 2.906915 |
| GO: 0060052~neurofilament cytoskeleton organization | 0.0025 | 2.602087 |
| GO: 0021612~facial nerve structural organization | 0.0025 | 2.602087 |
| GO: 0030334~regulation of cell migration | 0.002857 | 2.544101 |
| GO: 0030182~neuron differentiation | 0.002909 | 2.53628 |
| GO: 0021785~branchiomotor neuron axon guidance | 0.00304 | 2.51711 |
| GO: 0007155~cell adhesion | 0.004087 | 2.388544 |
| GO: 0003334~keratinocyte development | 0.004269 | 2.36972 |
| GO: 0021527~spinal cord association neuron differentiation* | 0.004269 | 2.36972 |
| GO: 0031175~neuron projection development | 0.004279 | 2.368667 |
| GO: 0000226~microtubule cytoskeleton organization | 0.006276 | 2.202289 |
| GO: 0043524~negative regulation of neuron apoptotic process | 0.007695 | 2.113782 |
| GO: 0045944~positive regulation of transcription from RNA | 0.00901 | 2.045267 |
| polymerase II promoter | | |
| GO: 0046328~regulation of JNK cascade | 0.009082 | 2.041814 |
| GO: 0009952~anterior/posterior pattern specification | 0.010692 | 1.970948 |
| GO: 0001764~neuron migration | 0.015065 | 1.822041 |
| GO: 0045105~intermediate filament polymerization or | 0.015206 | 1.817986 |
| depolymerization | | |
| GO: 0021937~cerebellar Purkinje cell-granule cell precursor cell | 0.015206 | 1.817986 |
| signaling involved in regulation of granule cell precursor cell | | |
| proliferation | | |
| GO: 0016477~cell migration | 0.015652 | 1.805433 |
| GO: 0051965~positive regulation of synapse assembly* | 0.016414 | 1.784785 |
| GO: 0021766~hippocampus development | 0.018345 | 1.73647 |
| GO: 0010976~positive regulation of neuron projection | 0.021426 | 1.669058 |
| development | | |
| GO: 0006366~transcription from RNA polymerase II promoter | 0.021426 | 1.669058 |
| GO: 0033693~neurofilament bundle assembly | 0.022723 | 1.643541 |
| GO: 0030036~actin cytoskeleton organization | 0.023573 | 1.627577 |
| GO: 0007420~brain development | 0.025098 | 1.60036 |
| GO: 0021510~spinal cord development* | 0.026163 | 1.582313 |
| GO: 0007166~cell surface receptor signaling pathway | 0.026432 | 1.577872 |
| GO: 0021559~trigeminal nerve development | 0.030182 | 1.520247 |
| GO: 0007417~central nervous system development | 0.030694 | 1.512948 |
| GO: 0045893~positive regulation of transcription, DNA- | 0.032606 | 1.486697 |
| templated | | |
| GO: 0010628~positive regulation of gene expression | 0.033528 | 1.47459 |
| GO: 0045666~positive regulation of neuron differentiation | 0.0443 | 1.353599 |
| GO: 0021637~trigeminal nerve structural organization | 0.044933 | 1.347438 |
| GO: 0045110~intermediate filament bundle assembly | 0.044933 | 1.347438 |
| GO: 0007623~circadian rhythm | 0.049775 | 1.302992 |
| GO: 0001657~ureteric bud development | 0.051923 | 1.284637 |
| GO: 0051152~positive regulation of smooth muscle cell | 0.052224 | 1.28213 |
| differentiation | | |
| GO: 0061303~cornea development in camera-type eye | 0.052224 | 1.28213 |
| GO: 0021517~ventral spinal cord development | 0.052224 | 1.28213 |
| GO: 0010629~negative regulation of gene expression | 0.052661 | 1.278512 |
| GO: 0009791~post-embryonic development | 0.056759 | 1.245969 |
| GO: 0007611~learning or memory | 0.057836 | 1.237799 |
| GO: 0071493~cellular response to UV-B | 0.05946 | 1.225774 |
| GO: 0048672~positive regulation of collateral sprouting | 0.05946 | 1.225774 |
| GO: 0007157~heterophilic cell-cell adhesion via plasma | 0.059857 | 1.222884 |
| membrane cell adhesion molecules | | |
| GO: 0048705~skeletal system morphogenesis | 0.063971 | 1.19402 |
| GO: 0048704~embryonic skeletal system morphogenesis | 0.066062 | 1.180048 |
| GO: 0061564~axon development | 0.066641 | 1.176256 |
| GO: 0043068~positive regulation of programmed cell death | 0.066641 | 1.176256 |
| GO: 0021796~cerebral cortex regionalization | 0.066641 | 1.176256 |
| GO: 0043066~negative regulation of apoptotic process | 0.06857 | 1.163863 |
| GO: 0007219~Notch signaling pathway | 0.070692 | 1.15063 |
| GO: 0043588~skin development | 0.072471 | 1.139836 |
| GO: 0050885~neuromuscular process controlling balance* | 0.072471 | 1.139836 |
| GO: 0010941~regulation of cell death | 0.073768 | 1.13213 |
| GO: 0045892~negative regulation of transcription, DNA- | 0.075978 | 1.119315 |
| templated | | |
| GO: 0048667~cell morphogenesis involved in neuron | 0.080841 | 1.092368 |
| differentiation | | |
| GO: 0021952~central nervous system projection neuron | 0.08786 | 1.056207 |
| axonogenesis | | |
| GO: 0021953~central nervous system neuron differentiation | 0.08786 | 1.056207 |
| GO: 1902287~semaphorin-plexin signaling pathway involved in | 0.094826 | 1.023071 |
| axon guidance | | |
| GO: 0021675~nerve development | 0.094826 | 1.023071 |
| GO: 0045669~positive regulation of osteoblast differentiation | 0.095152 | 1.02158 |

| | -log10 (p |
|---|---|
| Selected terms | value) |
| GO: 0007399~nervous system development | 11.97330193 |
| GO: 0048485~sympathetic nervous system development | 3.563092587 |
| GO: 0021527~spinal cord association neuron differentiation | 2.369720449 |
| GO: 0051965~positive regulation of synapse assembly | 1.784785447 |
| GO: 0021510~spinal cord development | 1.582312976 |
| GO: 0050885~neuromuscular process controlling balance | 1.139835583 |

Protocol 3 Cluster 5 Pathway

| | | -Log10 (P |
|---|---|---|
| All terms | PValue | value) |
| mmu05202: Transcriptional misregulation in cancer | 0.00213 | 2.671595 |
| mmu04010: MAPK signaling pathway | 0.012446 | 1.904977 |
| mmu04514: Cell adhesion molecules (CAMs) | 0.061348 | 1.2122 |

Protocol 3 Cluster 10 BioProcess

| All terms | PValue | -log10 (p value) |
|---|---|---|
| GO: 0007399~nervous system development* | 1.87E−12 | 11.72778 |
| GO: 0007409~axonogenesis | 7.01E−11 | 10.1544 |
| GO: 0007411~axon guidance | 5.06E−08 | 7.295624 |
| GO: 0031110~regulation of microtubule polymerization or | 7.14E−08 | 7.146048 |
| depolymerization | | |
| GO: 0031175~neuron projection development | 1.04E−05 | 4.98497 |
| GO: 0007268~chemical synaptic transmission* | 1.15E−05 | 4.939335 |
| GO: 0007275~multicellular organism development | 3.85E−05 | 4.414874 |
| GO: 0035418~protein localization to synapse | 3.91E−05 | 4.407784 |
| GO: 0030154~cell differentiation | 1.33E−04 | 3.874783 |
| GO: 0048846~axon extension involved in axon guidance | 2.20E−04 | 3.658501 |
| GO: 0060291~long-term synaptic potentiation | 3.63E−04 | 3.440272 |
| GO: 0007269~neurotransmitter secretion* | 3.91E−04 | 3.407973 |
| GO: 0007018~microtubule-based movement | 4.43E−04 | 3.353467 |
| GO: 0021828~gonadotrophin-releasing hormone neuronal | 4.67E−04 | 3.330831 |
| migration to the hypothalamus | | |
| GO: 1903375~facioacoustic ganglion development | 4.67E−04 | 3.330831 |
| GO: 0021675~nerve development | 5.09E−04 | 3.293423 |
| GO: 0030705~cytoskeleton-dependent intracellular | 6.42E−04 | 3.192718 |
| transport | | |
| GO: 0007019~microtubule depolymerization | 6.42E−04 | 3.192718 |
| GO: 1902285~semaphorin-plexin signaling pathway involved | 9.26E−04 | 3.033416 |
| in neuron projection guidance | | |
| GO: 0097491~sympathetic neuron projection guidance | 9.26E−04 | 3.033416 |
| GO: 0036486~ventral trunk neural crest cell migration | 9.26E−04 | 3.033416 |
| GO: 0072086~specification of loop of Henle identity | 9.26E−04 | 3.033416 |
| GO: 0097490~sympathetic neuron projection extension | 9.26E−04 | 3.033416 |
| GO: 0001764~neuron migration | 9.73E−04 | 3.01169 |
| GO: 0007155~cell adhesion | 0.001235 | 2.908248 |
| GO: 0007413~axonal fasciculation | 0.001386 | 2.858228 |
| GO: 0007612~learning | 0.001431 | 2.844298 |
| GO: 0061551~trigeminal ganglion development | 0.00153 | 2.815178 |
| GO: 0045773~positive regulation of axon extension | 0.001551 | 2.809395 |
| GO: 0007628~adult walking behavior* | 0.001863 | 2.729839 |
| GO: 0021637~trigeminal nerve structural organization | 0.002277 | 2.642694 |
| GO: 0007264~small GTPase mediated signal transduction | 0.003041 | 2.517019 |
| GO: 1901166~neural crest cell migration involved in | 0.003161 | 2.500169 |
| autonomic nervous system development | | |
| GO: 0008285~negative regulation of cell proliferation | 0.003477 | 2.45884 |
| GO: 0017157~regulation of exocytosis | 0.003662 | 2.436267 |
| GO: 0008088~axo-dendritic transport | 0.00418 | 2.378831 |
| GO: 0008045~motor neuron axon guidance | 0.005074 | 2.294647 |
| GO: 0061549~sympathetic ganglion development | 0.00533 | 2.273282 |
| GO: 0050885~neuromuscular process controlling balance* | 0.006042 | 2.218821 |
| GO: 0007568~aging | 0.006274 | 2.202455 |
| GO: 0021612~facial nerve structural organization | 0.006607 | 2.179965 |
| GO: 0060052~neurofilament cytoskeleton organization | 0.006607 | 2.179965 |
| GO: 0000226~microtubule cytoskeleton organization | 0.007236 | 2.140502 |
| GO: 0010976~positive regulation of neuron projection | 0.00798 | 2.097985 |
| development | | |
| GO: 0031115~negative regulation of microtubule | 0.008009 | 2.096403 |
| polymerization | | |
| GO: 0090129~positive regulation of synapse maturation | 0.008009 | 2.096403 |
| GO: 0016082~synaptic vesicle priming | 0.008009 | 2.096403 |
| GO: 0021785~branchiomotor neuron axon guidance | 0.008009 | 2.096403 |
| GO: 0014049~positive regulation of glutamate secretion | 0.008009 | 2.096403 |
| GO: 0010977~negative regulation of neuron projection | 0.008537 | 2.068692 |
| development | | |
| GO: 1904322~cellular response to forskolin | 0.009532 | 2.020807 |
| GO: 0035115~embryonic forelimb morphogenesis | 0.010276 | 1.988171 |
| GO: 0002027~regulation of heart rate | 0.010276 | 1.988171 |
| GO: 0008344~adult locomotory behavior* | 0.011067 | 1.955956 |
| GO: 0050808~synapse organization | 0.011081 | 1.955422 |
| GO: 1902287~semaphorin-plexin signaling pathway involved | 0.011173 | 1.951838 |
| in axon guidance | | |
| GO: 0051965~positive regulation of synapse assembly | 0.011625 | 1.934596 |
| GO: 0001656~metanephros development | 0.011921 | 1.923677 |
| GO: 0048489~synaptic vesicle transport* | 0.012928 | 1.888468 |
| GO: 0051493~regulation of cytoskeleton organization | 0.014795 | 1.829894 |
| GO: 0014059~regulation of dopamine secretion | 0.014795 | 1.829894 |
| GO: 0061029~eyelid development in camera-type eye | 0.014795 | 1.829894 |
| GO: 0043524~negative regulation of neuron apoptotic | 0.01572 | 1.803548 |
| process | | |
| GO: 0019233~sensory perception of pain* | 0.016039 | 1.794835 |
| GO: 0046426~negative regulation of JAK-STAT cascade | 0.016662 | 1.778279 |
| GO: 0045104~intermediate filament cytoskeleton | 0.01677 | 1.775473 |
| organization | | |
| GO: 2000679~positive regulation of transcription regulatory | 0.01677 | 1.775473 |
| region DNA binding | | |
| GO: 0007156~homophilic cell adhesion via plasma | 0.017078 | 1.767567 |
| membrane adhesion molecules | | |
| GO: 0008104~protein localization | 0.017468 | 1.757763 |
| GO: 0048661~positive regulation of smooth muscle cell | 0.018211 | 1.739661 |
| proliferation | | |
| GO: 0007416~synapse assembly | 0.018812 | 1.725555 |
| GO: 0007420~brain development | 0.019544 | 1.70899 |
| GO: 0050796~regulation of insulin secretion | 0.02111 | 1.675518 |
| GO: 0060048~cardiac muscle contraction | 0.022313 | 1.651434 |
| GO: 0072272~proximal/distal pattern formation involved in | 0.024951 | 1.602913 |
| metanephric nephron development | | |
| GO: 0014048~regulation of glutamate secretion | 0.024951 | 1.602913 |
| GO: 1904835~dorsal root ganglion morphogenesis | 0.024951 | 1.602913 |
| GO: 0045105~intermediate filament polymerization or | 0.024951 | 1.602913 |
| depolymerization | | |
| GO: 0021649~vestibulocochlear nerve structural | 0.024951 | 1.602913 |
| organization | | |
| GO: 0022409~positive regulation of cell-cell adhesion | 0.025695 | 1.590143 |
| GO: 0031290~retinal ganglion cell axon guidance | 0.025695 | 1.590143 |
| GO: 0007417~central nervous system development* | 0.025789 | 1.588561 |
| GO: 0007157~heterophilic cell-cell adhesion via plasma | 0.027495 | 1.56074 |
| membrane cell adhesion molecules | | |
| GO: 0045893~positive regulation of transcription, DNA- | 0.030326 | 1.518178 |
| templated | | |
| GO: 0061178~regulation of insulin secretion involved in | 0.033387 | 1.476419 |
| cellular response to glucose stimulus | | |
| GO: 0010842~retina layer formation | 0.033387 | 1.476419 |
| GO: 0007026~negative regulation of microtubule | 0.036127 | 1.442171 |
| depolymerization | | |
| GO: 0009987~cellular process | 0.036127 | 1.442171 |
| GO: 0048666~neuron development | 0.036363 | 1.439343 |
| GO: 0045920~negative regulation of exocytosis | 0.037193 | 1.429539 |
| GO: 0033693~neurofilament bundle assembly | 0.037193 | 1.429539 |
| GO: 0032387~negative regulation of intracellular transport | 0.037193 | 1.429539 |
| GO: 0097374~sensory neuron axon guidance* | 0.037193 | 1.429539 |
| GO: 0021772~olfactory bulb development | 0.03895 | 1.409495 |
| GO: 0030001~metal ion transport | 0.03895 | 1.409495 |
| GO: 0045666~positive regulation of neuron differentiation | 0.040861 | 1.388688 |
| GO: 0032922~circadian regulation of gene expression | 0.041282 | 1.38424 |
| GO: 0048843~negative regulation of axon extension | 0.041854 | 1.378265 |
| involved in axon guidance | | |
| GO: 0050919~negative chemotaxis | 0.041854 | 1.378265 |
| GO: 0048675~axon extension | 0.041854 | 1.378265 |
| GO: 0072659~protein localization to plasma membrane | 0.042992 | 1.366608 |
| GO: 0007517~muscle organ development | 0.044738 | 1.349323 |
| GO: 0061024~membrane organization | 0.044738 | 1.349323 |
| GO: 0048812~neuron projection morphogenesis | 0.044738 | 1.349323 |
| GO: 0050806~positive regulation of synaptic transmission* | 0.044836 | 1.34837 |
| GO: 0045921~positive regulation of exocytosis | 0.047895 | 1.31971 |
| GO: 0030100~regulation of endocytosis | 0.047895 | 1.31971 |
| GO: 0048169~regulation of long-term neuronal synaptic | 0.047895 | 1.31971 |
| plasticity | | |
| GO: 0007052~mitotic spindle organization | 0.047895 | 1.31971 |
| GO: 0038084~vascular endothelial growth factor signaling | 0.049282 | 1.307312 |
| pathway | | |
| GO: 0007423~sensory organ development* | 0.049282 | 1.307312 |
| GO: 0006890~retrograde vesicle-mediated transport, Golgi | 0.051027 | 1.292197 |
| to ER | | |
| GO: 0043392~negative regulation of DNA binding | 0.051027 | 1.292197 |
| GO: 0010975~regulation of neuron projection development | 0.054231 | 1.265751 |
| GO: 0009791~post-embryonic development | 0.055564 | 1.25521 |
| GO: 0040008~regulation of growth | 0.055934 | 1.252325 |
| GO: 0045664~regulation of neuron differentiation | 0.057504 | 1.240301 |
| GO: 0045777~positive regulation of blood pressure | 0.057504 | 1.240301 |
| GO: 0060666~dichotomous subdivision of terminal units | 0.06122 | 1.213108 |
| involved in salivary gland branching | | |
| GO: 0006903~vesicle targeting | 0.06122 | 1.213108 |
| GO: 0033602~negative regulation of dopamine secretion | 0.06122 | 1.213108 |
| GO: 0019896~axonal transport of mitochondrion | 0.06122 | 1.213108 |
| GO: 0007188~adenylate cyclase-modulating G-protein | 0.067716 | 1.169308 |
| coupled receptor signaling pathway | | |
| GO: 0045665~negative regulation of neuron differentiation | 0.070503 | 1.151793 |
| GO: 0047497~mitochondrion transport along microtubule | 0.073008 | 1.136627 |
| GO: 0090036~regulation of protein kinase C signaling | 0.073008 | 1.136627 |
| GO: 0014904~myotube cell development | 0.073008 | 1.136627 |
| GO: 0045110~intermediate filament bundle assembly | 0.073008 | 1.136627 |
| GO: 0010882~regulation of cardiac muscle contraction by | 0.073008 | 1.136627 |
| calcium ion signaling | | |
| GO: 0060385~axonogenesis involved in innervation | 0.073008 | 1.136627 |
| GO: 0031133~regulation of axon diameter | 0.073008 | 1.136627 |
| GO: 0032410~negative regulation of transporter activity | 0.073008 | 1.136627 |
| GO: 0016358~dendrite development | 0.07483 | 1.125922 |
| GO: 0048511~rhythmic process | 0.077855 | 1.108713 |
| GO: 0021942~radial glia guided migration of Purkinje cell | 0.08465 | 1.072374 |
| GO: 1901018~positive regulation of potassium ion | 0.08465 | 1.072374 |
| transmembrane transporter activity | | |
| GO: 2000144~positive regulation of DNA-templated | 0.08465 | 1.072374 |
| transcription, initiation | | |
| GO: 0048841~regulation of axon extension involved in axon | 0.08465 | 1.072374 |
| guidance | | |
| GO: 0055119~relaxation of cardiac muscle | 0.08465 | 1.072374 |
| GO: 0048701~embryonic cranial skeleton morphogenesis | 0.08592 | 1.065904 |
| GO: 0007613~memory | 0.086591 | 1.062529 |
| GO: 0031623~receptor internalization | 0.089721 | 1.047106 |
| GO: 0007017~microtubule-based process | 0.09357 | 1.028862 |
| GO: 0010881~regulation of cardiac muscle contraction by | 0.096145 | 1.017071 |
| regulation of the release of sequestered calcium ion | | |
| GO: 0048672~positive regulation of collateral sprouting | 0.096145 | 1.017071 |
| GO: 0071514~genetic imprinting | 0.096145 | 1.017071 |
| GO: 0031987~locomotion involved in locomotory behavior* | 0.096145 | 1.017071 |
| GO: 2000480~negative regulation of cAMP-dependent | 0.096145 | 1.017071 |
| protein kinase activity | | |
| GO: 0007162~negative regulation of cell adhesion | 0.097467 | 1.011143 |

| | -log10 (p |
|---|---|
| Selected terms | value) |
| GO: 0007399~nervous system development | 11.72777689 |
| GO: 0007268~chemical synaptic transmission | 4.939335336 |
| GO: 0007269~neurotransmitter secretion | 3.407972537 |
| GO: 0007628~adult walking behavior | 2.729838662 |
| GO: 0050885~neuromuscular process controlling balance | 2.218821315 |
| GO: 0008344~adult locomotory behavior | 1.955956297 |
| GO: 0048489~synaptic vesicle transport | 1.888468476 |
| GO: 0007417~central nervous system development | 1.588560952 |
| GO: 0097374~sensory neuron axon guidance | 1.429539142 |
| GO: 0050806~positive regulation of synaptic transmission | 1.348370263 |
| GO: 0007423~sensory organ development | 1.307311861 |
| GO: 0031987~locomotion involved in locomotory behavior | 1.017071415 |

Protocol 3 Cluster 10 Pathway

| | | -log10 (p |
|---|---|---|
| Term | PValue | value) |
| mmu04728: Dopaminergic synapse* | 3.57E−06 | 5.44747 |
| mmu04360: Axon guidance | 1.95E−05 | 4.709738 |
| mmu04713: Circadian entrainment | 1.49E−04 | 3.828167 |
| mmu04724: Glutamatergic synapse* | 4.00E−04 | 3.39809 |
| mmu04024: cAMP signaling pathway | 5.05E−04 | 3.296714 |
| mmu05202: Transcriptional misregulation in cancer | 7.10E−04 | 3.148907 |
| mmu04015: Rap1 signaling pathway | 9.16E−04 | 3.037972 |
| mmu04911: Insulin secretion | 0.003404 | 2.467995 |
| mmu04727: GABAergic synapse | 0.003579 | 2.446297 |
| mmu04014: Ras signaling pathway | 0.005551 | 2.255594 |
| mmu04261: Adrenergic signaling in cardiomyocytes | 0.006569 | 2.182486 |
| mmu04723: Retrograde endocannabinoid signaling* | 0.007317 | 2.135694 |
| mmu04720: Long-term potentiation | 0.007562 | 2.121388 |
| mmu05031: Amphetamine addiction* | 0.00797 | 2.09853 |
| mmu04010: MAPK signaling pathway | 0.009473 | 2.023527 |
| mmu04971: Gastric acid secretion | 0.010231 | 1.990063 |
| mmu04725: Cholinergic synapse* | 0.010702 | 1.970529 |
| mmu04722: Neurotrophin signaling pathway* | 0.014554 | 1.837021 |
| mmu05014: Amyotrophic lateral sclerosis (ALS) | 0.022174 | 1.654153 |
| mmu05032: Morphine addiction | 0.0241 | 1.617983 |
| mmu04310: Wnt signaling pathway | 0.025518 | 1.593148 |
| mmu04915: Estrogen signaling pathway | 0.028528 | 1.544724 |
| mmu05034: Alcoholism | 0.032129 | 1.493103 |
| mmu04921: Oxytocin signaling pathway | 0.032177 | 1.492453 |
| mmu04730: Long-term depression | 0.035238 | 1.452995 |
| mmu05214: Glioma | 0.041361 | 1.383411 |
| mmu04550: Signaling pathways regulating pluripotency of stem | | |
| cells | 0.080478 | 1.094325 |
| mmu04012: ErbB signaling pathway | 0.083629 | 1.077642 |
| mmu04912: GnRH signaling pathway | 0.085871 | 1.066155 |
| mmu05205: Proteoglycans in cancer | 0.092204 | 1.035251 |
| mmu05200: Pathways in cancer | 0.094756 | 1.023391 |

| | -log10 (p |
|---|---|
| Selected terms | value) |
| mmu04728: Dopaminergic synapse | 5.447470182 |
| mmu04724: Glutamatergic synapse | 3.398089783 |
| mmu04723: Retrograde endocannabinoid signaling | 2.135694291 |
| mmu05031: Amphetamine addiction | 2.09852978 |
| mmu04725: Cholinergic synapse | 1.970528575 |
| mmu04722: Neurotrophin signaling pathway | 1.837021227 |

TABLE 3
GO categories for 78 common genes.
Biological Process Common 78 gene (GOTERM_BP_DIRECT)

| | | | | |
|---|---|---|---|---|
| GO: 0007275~multicellular organism | 28 | 36.36364 | 6.71E−18 | 17.17348 |
| development* | | | | |
| GO: 0045893~positive regulation of | 19 | 24.67532 | 4.41E−13 | 12.35587 |
| transcription, DNA-templated* | | | | |
| GO: 0045944~positive regulation of | 20 | 25.97403 | 4.46E−10 | 9.350493 |
| transcription from RNA polymerase II | | | | |
| promoter | | | | |
| GO: 0006351~transcription, DNA- | 26 | 33.76623 | 7.69E−10 | 9.114059 |
| templated | | | | |
| GO: 0006355~regulation of transcription, | 28 | 36.36364 | 1.44E−09 | 8.841487 |
| DNA-templated* | | | | |
| GO: 0000122~negative regulation of | 17 | 22.07792 | 2.00E−09 | 8.699345 |
| transcription from RNA polymerase II | | | | |
| promoter | | | | |
| GO: 0060045~positive regulation of | 6 | 7.792208 | 1.29E−08 | 7.88943 |
| cardiac muscle cell proliferation | | | | |
| GO: 0060021~palate development | 8 | 10.38961 | 1.57E−08 | 7.802978 |
| GO: 0030509~BMP signaling pathway* | 8 | 10.38961 | 1.85E−08 | 7.731994 |
| GO: 0003151~outflow tract | 7 | 9.090909 | 2.88E−08 | 7.540434 |
| morphogenesis | | | | |
| GO: 0001947~heart looping | 7 | 9.090909 | 6.71E−08 | 7.17305 |
| GO: 0030326~embryonic limb | 7 | 9.090909 | 7.40E−08 | 7.130738 |
| morphogenesis | | | | |
| GO: 0035115~embryonic forelimb | 6 | 7.792208 | 1.40E−07 | 6.854915 |
| morphogenesis | | | | |
| GO: 0042472~inner ear morphogenesis | 7 | 9.090909 | 1.66E−07 | 6.779498 |
| GO: 0045892~negative regulation of | 13 | 16.88312 | 5.19E−07 | 6.284631 |
| transcription, DNA-templated | | | | |
| GO: 0001701~in utero embryonic | 10 | 12.98701 | 7.71E−07 | 6.112814 |
| development | | | | |
| GO: 0035050~embryonic heart tube | 5 | 6.493506 | 1.06E−06 | 5.976123 |
| development | | | | |
| GO: 0090090~negative regulation of | 7 | 9.090909 | 1.32E−06 | 5.878684 |
| canonical Wnt signaling pathway | | | | |
| GO: 0060536~cartilage morphogenesis | 4 | 5.194805 | 1.33E−06 | 5.875983 |
| GO: 0007267~cell-cell signaling | 7 | 9.090909 | 1.40E−06 | 5.853773 |
| GO: 0003007~heart morphogenesis | 6 | 7.792208 | 2.62E−06 | 5.580989 |
| GO: 0042733~embryonic digit | 6 | 7.792208 | 3.06E−06 | 5.514294 |
| morphogenesis | | | | |
| GO: 0090263~positive regulation of | 6 | 7.792208 | 3.30E−06 | 5.481751 |
| canonical Wnt signaling pathway* | | | | |
| GO: 0061180~mammary gland epithelium | 4 | 5.194805 | 6.21E−06 | 5.206821 |
| development | | | | |
| GO: 0030901~midbrain development | 5 | 6.493506 | 1.04E−05 | 4.981355 |
| GO: 0060070~canonical Wnt signaling | 6 | 7.792208 | 1.34E−05 | 4.873579 |
| pathway* | | | | |
| GO: 0030514~negative regulation of BMP | 5 | 6.493506 | 1.84E−05 | 4.736254 |
| signaling pathway | | | | |
| GO: 0045599~negative regulation of fat | 5 | 6.493506 | 2.18E−05 | 4.662062 |
| cell differentiation | | | | |
| GO: 0048468~cell development | 5 | 6.493506 | 2.36E−05 | 4.626192 |
| GO: 0007507~heart development | 8 | 10.38961 | 3.01E−05 | 4.521453 |
| GO: 0001709~cell fate determination* | 4 | 5.194805 | 3.02E−05 | 4.520055 |
| GO: 0061184~positive regulation of | 3 | 3.896104 | 3.46E−05 | 4.460588 |
| dermatome development | | | | |
| GO: 0009952~anterior/posterior pattern | 6 | 7.792208 | 4.07E−05 | 4.39031 |
| specification | | | | |
| GO: 0010628~positive regulation of gene | 9 | 11.68831 | 6.25E−05 | 4.203927 |
| expression | | | | |
| GO: 0003148~outflow tract septum | 4 | 5.194805 | 7.38E−05 | 4.131907 |
| morphogenesis | | | | |
| GO: 0030182~neuron differentiation* | 6 | 7.792208 | 7.42E−05 | 4.129762 |
| GO: 0016055~Wnt signaling pathway* | 7 | 9.090909 | 8.83E−05 | 4.053797 |
| GO: 0033278~cell proliferation in | 3 | 3.896104 | 1.15E−04 | 3.939632 |
| midbrain | | | | |
| GO: 0060676~ureteric bud formation | 3 | 3.896104 | 1.15E−04 | 3.939632 |
| GO: 0051891~positive regulation of | 3 | 3.896104 | 1.15E−04 | 3.939632 |
| cardioblast differentiation | | | | |
| GO: 0007411~axon guidance | 6 | 7.792208 | 1.57E−04 | 3.80272 |
| GO: 0001837~epithelial to mesenchymal | 4 | 5.194805 | 1.61E−04 | 3.792816 |
| transition | | | | |
| GO: 0061312~BMP signaling pathway | 3 | 3.896104 | 1.72E−04 | 3.764502 |
| involved in heart development | | | | |
| GO: 0060349~bone morphogenesis | 4 | 5.194805 | 1.77E−04 | 3.751125 |
| GO: 0051216~cartilage development | 5 | 6.493506 | 1.79E−04 | 3.746157 |
| GO: 0071773~cellular response to BMP | 4 | 5.194805 | 2.32E−04 | 3.633878 |
| stimulus* | | | | |
| GO: 0060575~intestinal epithelial cell | 3 | 3.896104 | 2.40E−04 | 3.619335 |
| differentiation | | | | |
| GO: 0030513~positive regulation of BMP | 4 | 5.194805 | 2.53E−04 | 3.59715 |
| signaling pathway | | | | |
| GO: 0055007~cardiac muscle cell | 4 | 5.194805 | 2.74E−04 | 3.561487 |
| differentiation | | | | |
| GO: 0010629~negative regulation of gene | 7 | 9.090909 | 2.89E−04 | 3.538389 |
| expression | | | | |
| GO: 0035239~tube morphogenesis | 3 | 3.896104 | 3.20E−04 | 3.495357 |
| GO: 1904948~midbrain dopaminergic | 3 | 3.896104 | 3.20E−04 | 3.495357 |
| neuron differentiation | | | | |
| GO: 0033077~T cell differentiation in | 4 | 5.194805 | 3.21E−04 | 3.493129 |
| thymus | | | | |
| GO: 0060325~face morphogenesis | 4 | 5.194805 | 3.46E−04 | 3.46033 |
| GO: 0048565~digestive tract development | 4 | 5.194805 | 4.01E−04 | 3.397264 |
| GO: 0008284~positive regulation of cell | 9 | 11.68831 | 5.04E−04 | 3.297631 |
| proliferation* | | | | |
| GO: 0009887~organ morphogenesis | 5 | 6.493506 | 5.33E−04 | 3.27359 |
| GO: 0008584~male gonad development | 5 | 6.493506 | 5.90E−04 | 3.229095 |
| GO: 0001649~osteoblast differentiation | 5 | 6.493506 | 6.10E−04 | 3.214552 |
| GO: 0060394~negative regulation of | 3 | 3.896104 | 6.24E−04 | 3.205034 |
| pathway-restricted SMAD protein | | | | |
| phosphorylation | | | | |
| GO: 0043066~negative regulation of | 9 | 11.68831 | 6.70E−04 | 3.174014 |
| apoptotic process | | | | |
| GO: 0003198~epithelial to mesenchymal | 3 | 3.896104 | 8.81E−04 | 3.055221 |
| transition involved in endocardial cushion | | | | |
| formation | | | | |
| GO: 0003215~cardiac right ventricle | 3 | 3.896104 | 8.81E−04 | 3.055221 |
| morphogenesis | | | | |
| GO: 0045668~negative regulation of | 4 | 5.194805 | 9.35E−04 | 3.02923 |
| osteoblast differentiation | | | | |
| GO: 0060413~atrial septum | 3 | 3.896104 | 0.001025 | 2.989234 |
| morphogenesis | | | | |
| GO: 0035136~forelimb morphogenesis | 3 | 3.896104 | 0.00118 | 2.928045 |
| GO: 2000679~positive regulation of | 3 | 3.896104 | 0.001346 | 2.871012 |
| transcription regulatory region DNA | | | | |
| binding | | | | |
| GO: 0001706~endoderm formation | 3 | 3.896104 | 0.001346 | 2.871012 |
| GO: 0003203~endocardial cushion | 3 | 3.896104 | 0.001522 | 2.817613 |
| morphogenesis | | | | |
| GO: 0014032~neural crest cell | 3 | 3.896104 | 0.001522 | 2.817613 |
| development | | | | |
| GO: 0048485~sympathetic nervous | 3 | 3.896104 | 0.001522 | 2.817613 |
| system development | | | | |
| GO: 0090398~cellular senescence | 3 | 3.896104 | 0.002112 | 2.675273 |
| GO: 0043627~response to estrogen | 4 | 5.194805 | 0.002175 | 2.662625 |
| GO: 0006357~regulation of transcription | 7 | 9.090909 | 0.002352 | 2.628606 |
| from RNA polymerase II promoter | | | | |
| GO: 0023019~signal transduction involved | 3 | 3.896104 | 0.002794 | 2.553779 |
| in regulation of gene expression | | | | |
| GO: 0042474~middle ear morphogenesis | 3 | 3.896104 | 0.003041 | 2.516947 |
| GO: 0048646~anatomical structure | 3 | 3.896104 | 0.003041 | 2.516947 |
| formation involved in morphogenesis | | | | |
| GO: 0009954~proximal/distal pattern | 3 | 3.896104 | 0.003565 | 2.447886 |
| formation | | | | |
| GO: 2001234~negative regulation of | 3 | 3.896104 | 0.003842 | 2.415419 |
| apoptotic signaling pathway | | | | |
| GO: 0043392~negative regulation of DNA | 3 | 3.896104 | 0.004425 | 2.354112 |
| binding | | | | |
| GO: 0048738~cardiac muscle tissue | 3 | 3.896104 | 0.004425 | 2.354112 |
| development | | | | |
| GO: 0071542~dopaminergic neuron | 3 | 3.896104 | 0.004425 | 2.354112 |
| differentiation | | | | |
| GO: 0021983~pituitary gland | 3 | 3.896104 | 0.00473 | 2.325105 |
| development | | | | |
| GO: 0048663~neuron fate commitment* | 3 | 3.896104 | 0.00537 | 2.270023 |
| GO: 0035116~embryonic hindlimb | 3 | 3.896104 | 0.005704 | 2.243826 |
| morphogenesis | | | | |
| GO: 0030336~negative regulation of cell | 4 | 5.194805 | 0.005763 | 2.239347 |
| migration | | | | |
| GO: 0001707~mesoderm formation | 3 | 3.896104 | 0.0064 | 2.193852 |
| GO: 0003218~cardiac left ventricle | 2 | 2.597403 | 0.006846 | 2.164558 |
| formation | | | | |
| GO: 0090427~activation of meiosis | 2 | 2.597403 | 0.006846 | 2.164558 |
| GO: 0003281~ventricular septum | 3 | 3.896104 | 0.007511 | 2.124276 |
| development | | | | |
| GO: 0030879~mammary gland | 3 | 3.896104 | 0.007511 | 2.124276 |
| development | | | | |
| GO: 0045766~positive regulation of | 4 | 5.194805 | 0.008297 | 2.081104 |
| angiogenesis | | | | |
| GO: 0045787~positive regulation of cell | 3 | 3.896104 | 0.008298 | 2.081044 |
| cycle* | | | | |
| GO: 0048706~embryonic skeletal system | 3 | 3.896104 | 0.009976 | 2.001058 |
| development | | | | |
| GO: 0003166~bundle of His development | 2 | 2.597403 | 0.010252 | 1.9892 |
| GO: 0035880~embryonic nail plate | 2 | 2.597403 | 0.010252 | 1.9892 |
| morphogenesis | | | | |
| GO: 2001055~positive regulation of | 2 | 2.597403 | 0.010252 | 1.9892 |
| mesenchymal cell apoptotic process | | | | |
| GO: 0048469~cell maturation | 3 | 3.896104 | 0.010417 | 1.982267 |
| GO: 0035019~somatic stem cell | 3 | 3.896104 | 0.010866 | 1.963913 |
| population maintenance | | | | |
| GO: 0001658~branching involved in | 3 | 3.896104 | 0.011791 | 1.928448 |
| ureteric bud morphogenesis | | | | |
| GO: 0001657~ureteric bud development | 3 | 3.896104 | 0.011791 | 1.928448 |
| GO: 0006366~transcription from RNA | 4 | 5.194805 | 0.01185 | 1.926282 |
| polymerase II promoter | | | | |
| GO: 0003219~cardiac right ventricle | 2 | 2.597403 | 0.013646 | 1.864993 |
| formation | | | | |
| GO: 1904954~canonical Wnt signaling | 2 | 2.597403 | 0.013646 | 1.864993 |
| pathway involved in midbrain | | | | |
| dopaminergic neuron differentiation | | | | |
| GO: 0021533~cell differentiation in | 2 | 2.597403 | 0.013646 | 1.864993 |
| hindbrain | | | | |
| GO: 0003229~ventricular cardiac muscle | 2 | 2.597403 | 0.013646 | 1.864993 |
| tissue development | | | | |
| GO: 0060596~mammary placode | 2 | 2.597403 | 0.013646 | 1.864993 |
| formation | | | | |
| GO: 0072210~metanephric nephron | 2 | 2.597403 | 0.013646 | 1.864993 |
| development | | | | |
| GO: 0030512~negative regulation of | 3 | 3.896104 | 0.014763 | 1.830822 |
| transforming growth factor beta receptor | | | | |
| signaling pathway | | | | |
| GO: 0048538~thymus development | 3 | 3.896104 | 0.015287 | 1.815685 |
| GO: 0030154~cell differentiation | 8 | 10.38961 | 0.016975 | 1.770186 |
| GO: 0060923~cardiac muscle cell fate | 2 | 2.597403 | 0.017029 | 1.768815 |
| commitment | | | | |
| GO: 0010463~mesenchymal cell | 2 | 2.597403 | 0.017029 | 1.768815 |
| proliferation | | | | |
| GO: 0003310~pancreatic A cell | 2 | 2.597403 | 0.017029 | 1.768815 |
| differentiation | | | | |
| GO: 0042475~odontogenesis of dentin- | 3 | 3.896104 | 0.019756 | 1.704299 |
| containing tooth | | | | |
| GO: 0022008~neurogenesis | 3 | 3.896104 | 0.020949 | 1.678833 |
| GO: 0045669~positive regulation of | 3 | 3.896104 | 0.022794 | 1.642184 |
| osteoblast differentiation | | | | |
| GO: 0043433~negative regulation of | 3 | 3.896104 | 0.023423 | 1.630356 |
| sequence-specific DNA binding | | | | |
| transcription factor activity | | | | |
| GO: 0060231~mesenchymal to epithelial | 2 | 2.597403 | 0.02376 | 1.62415 |
| transition | | | | |
| GO: 0003180~aortic valve morphogenesis | 2 | 2.597403 | 0.02376 | 1.62415 |
| GO: 0071371~cellular response to | 2 | 2.597403 | 0.02376 | 1.62415 |
| gonadotropin stimulus | | | | |
| GO: 0045165~cell fate commitment | 3 | 3.896104 | 0.025354 | 1.595962 |
| GO: 0055009~atrial cardiac muscle tissue | 2 | 2.597403 | 0.027109 | 1.566889 |
| morphogenesis | | | | |
| GO: 0060485~mesenchyme development | 2 | 2.597403 | 0.027109 | 1.566889 |
| GO: 0042048~olfactory behavior | 2 | 2.597403 | 0.027109 | 1.566889 |
| GO: 0036302~atrioventricular canal | 2 | 2.597403 | 0.027109 | 1.566889 |
| development | | | | |
| GO: 0045665~negative regulation of | 3 | 3.896104 | 0.028025 | 1.552459 |
| neuron differentiation | | | | |
| GO: 0060395~SMAD protein signal | 3 | 3.896104 | 0.02871 | 1.541974 |
| transduction | | | | |
| GO: 0060290~transdifferentiation | 2 | 2.597403 | 0.030446 | 1.516467 |
| GO: 0003309~type B pancreatic cell | 2 | 2.597403 | 0.030446 | 1.516467 |
| differentiation | | | | |
| GO: 0000902~cell morphogenesis | 3 | 3.896104 | 0.031515 | 1.501479 |
| GO: 0007613~memory | 3 | 3.896104 | 0.032957 | 1.482049 |
| GO: 0061371~determination of heart | 2 | 2.597403 | 0.033772 | 1.47144 |
| left/right asymmetry | | | | |
| GO: 2000727~positive regulation of | 2 | 2.597403 | 0.033772 | 1.47144 |
| cardiac muscle cell differentiation | | | | |
| GO: 0042118~endothelial cell activation | 2 | 2.597403 | 0.033772 | 1.47144 |
| GO: 0003149~membranous septum | 2 | 2.597403 | 0.033772 | 1.47144 |
| morphogenesis | | | | |
| GO: 0030097~hemopoiesis | 3 | 3.896104 | 0.035168 | 1.453852 |
| GO: 0030900~forebrain development | 3 | 3.896104 | 0.035168 | 1.453852 |
| GO: 0035413~positive regulation of | 2 | 2.597403 | 0.037087 | 1.430777 |
| catenin import into nucleus | | | | |
| GO: 0007420~brain development | 4 | 5.194805 | 0.037997 | 1.420251 |
| GO: 0001823~mesonephros development | 2 | 2.597403 | 0.040391 | 1.393718 |
| GO: 0008285~negative regulation of cell | 5 | 6.493506 | 0.042643 | 1.370155 |
| proliferation | | | | |
| GO: 0060044~negative regulation of | 2 | 2.597403 | 0.043683 | 1.359684 |
| cardiac muscle cell proliferation | | | | |
| GO: 2000678~negative regulation of | 2 | 2.597403 | 0.043683 | 1.359684 |
| transcription regulatory region DNA | | | | |
| binding | | | | |
| GO: 0010717~regulation of epithelial to | 2 | 2.597403 | 0.043683 | 1.359684 |
| mesenchymal transition | | | | |
| GO: 0021527~spinal cord association | 2 | 2.597403 | 0.043683 | 1.359684 |
| neuron differentiation* | | | | |
| GO: 0003197~endocardial cushion | 2 | 2.597403 | 0.043683 | 1.359684 |
| development | | | | |
| GO: 0060444~branching involved in | 2 | 2.597403 | 0.046965 | 1.328228 |
| mammary gland duct morphogenesis | | | | |
| GO: 2000178~negative regulation of | 2 | 2.597403 | 0.046965 | 1.328228 |
| neural precursor cell proliferation | | | | |
| GO: 0043586~tongue development | 2 | 2.597403 | 0.046965 | 1.328228 |
| GO: 0001525~angiogenesis | 4 | 5.194805 | 0.0488 | 1.311581 |
| GO: 0003181~atrioventricular valve | 2 | 2.597403 | 0.050235 | 1.298993 |
| morphogenesis | | | | |
| GO: 0051091~positive regulation of | 3 | 3.896104 | 0.052142 | 1.282816 |
| sequence-specific DNA binding | | | | |
| transcription factor activity | | | | |
| GO: 0050872~white fat cell differentiation | 2 | 2.597403 | 0.053494 | 1.271692 |
| GO: 0001655~urogenital system | 2 | 2.597403 | 0.053494 | 1.271692 |
| development | | | | |
| GO: 0048596~embryonic camera-type eye | 2 | 2.597403 | 0.056743 | 1.246091 |
| morphogenesis | | | | |
| GO: 0045596~negative regulation of cell | 3 | 3.896104 | 0.058334 | 1.234077 |
| differentiation | | | | |
| GO: 0032331~negative regulation of | 2 | 2.597403 | 0.05998 | 1.221994 |
| chondrocyte differentiation | | | | |
| GO: 0060216~definitive hemopoiesis | 2 | 2.597403 | 0.05998 | 1.221994 |
| GO: 0014898~cardiac muscle hypertrophy | 2 | 2.597403 | 0.05998 | 1.221994 |
| in response to stress | | | | |
| GO: 0021542~dentate gyrus development | 2 | 2.597403 | 0.05998 | 1.221994 |
| GO: 0051145~smooth muscle cell | 2 | 2.597403 | 0.05998 | 1.221994 |
| differentiation | | | | |
| GO: 0003279~cardiac septum | 2 | 2.597403 | 0.063206 | 1.19924 |
| development | | | | |
| GO: 0009888~tissue development | 2 | 2.597403 | 0.063206 | 1.19924 |
| GO: 0060065~uterus development | 2 | 2.597403 | 0.063206 | 1.19924 |
| GO: 0061098~positive regulation of | 2 | 2.597403 | 0.066422 | 1.17769 |
| protein tyrosine kinase activity | | | | |
| GO: 0001764~neuron migration | 3 | 3.896104 | 0.067596 | 1.170077 |
| GO: 0048557~embryonic digestive tract | 2 | 2.597403 | 0.069626 | 1.157226 |
| morphogenesis | | | | |
| GO: 0045648~positive regulation of | 2 | 2.597403 | 0.069626 | 1.157226 |
| erythrocyte differentiation | | | | |
| GO: 0001822~kidney development | 3 | 3.896104 | 0.073373 | 1.134465 |
| GO: 0090190~positive regulation of | 2 | 2.597403 | 0.076003 | 1.119168 |
| branching involved in ureteric bud | | | | |
| morphogenesis | | | | |
| GO: 0071158~positive regulation of cell | 2 | 2.597403 | 0.079175 | 1.10141 |
| cycle arrest | | | | |
| GO: 0060425~lung morphogenesis | 2 | 2.597403 | 0.079175 | 1.10141 |
| GO: 0048843~negative regulation of axon | 2 | 2.597403 | 0.085488 | 1.068096 |
| extension involved in axon guidance | | | | |
| GO: 0010667~negative regulation of | 2 | 2.597403 | 0.085488 | 1.068096 |
| cardiac muscle cell apoptotic process | | | | |
| GO: 0042476~odontogenesis | 2 | 2.597403 | 0.088628 | 1.052429 |
| GO: 0002092~positive regulation of | 2 | 2.597403 | 0.088628 | 1.052429 |
| receptor internalization | | | | |
| GO: 0010468~regulation of gene | 4 | 5.194805 | 0.088997 | 1.050625 |
| expression | | | | |
| GO: 0045638~negative regulation of | 2 | 2.597403 | 0.091758 | 1.037358 |
| myeloid cell differentiation | | | | |
| GO: 0090102~cochlea development | 2 | 2.597403 | 0.094877 | 1.02284 |
| GO: 0001819~positive regulation of | 2 | 2.597403 | 0.094877 | 1.02284 |
| cytokine production | | | | |
| GO: 0045880~positive regulation of | 2 | 2.597403 | 0.097985 | 1.00884 |
| smoothened signaling pathway | | | | |
| GO: 0007569~cell aging | 2 | 2.597403 | 0.097985 | 1.00884 |

| | -Log10 (P |
|---|---|
| Selected terms | value) |
| GO: 0007275~multicellular organism development | 17.17348022 |
| GO: 0045893~positive regulation of transcription, DNA-templated | 12.3558749 |
| GO: 0006355~regulation of transcription, DNA-templated | 8.841487007 |
| GO: 0030509~BMP signaling pathway | 7.731993875 |
| GO: 0090263~positive regulation of canonical Wnt signaling pathway | 5.481751155 |
| GO: 0060070~canonical Wnt signaling pathway | 4.873578838 |
| GO: 0001709~cell fate determination | 4.520055165 |
| GO: 0030182~neuron differentiation | 4.129761859 |
| GO: 0016055~Wnt signaling pathway | 4.053797336 |
| GO: 0071773~cellular response to BMP stimulus | 3.633878092 |
| GO: 0008284~positive regulation of cell proliferation | 3.297630738 |
| GO: 0048663~neuron fate commitment | 2.270023078 |
| GO: 0045787~positive regulation of cell cycle | 2.081043731 |
| GO: 0021527~spinal cord association neuron differentiation | 1.35968443 |

Mol Process Common 78 gene

| | | | | |
|---|---|---|---|---|
| GO: 0043565~sequence-specific DNA binding* | 22 | 28.57143 | 2.41E−16 | 15.61727 |
| GO: 0003677~DNA binding | 26 | 33.76623 | 1.21E−10 | 9.917124 |
| GO: 0000977~RNA polymerase II regulatory region | 11 | 14.28571 | 2.23E−09 | 8.651906 |
| sequence-specific DNA binding | | | | |
| GO: 0044212~transcription regulatory region DNA binding | 10 | 12.98701 | 5.97E−08 | 7.224043 |
| GO: 0005515~protein binding* | 33 | 42.85714 | 1.07E−07 | 6.968859 |
| GO: 0003700~transcription factor activity, sequence- | 15 | 19.48052 | 6.56E−07 | 6.183087 |
| specific DNA binding | | | | |
| GO: 0001085~RNA polymerase II transcription factor | 6 | 7.792208 | 9.56E−07 | 6.019699 |
| binding | | | | |
| GO: 0001228~transcriptional activator activity, RNA | 7 | 9.090909 | 1.10E−06 | 5.958406 |
| polymerase II transcription regulatory region sequence- | | | | |
| specific binding | | | | |
| GO: 0001158~enhancer sequence-specific DNA binding | 5 | 6.493506 | 1.45E−06 | 5.838415 |
| GO: 0008134~transcription factor binding | 10 | 12.98701 | 1.51E−06 | 5.820432 |
| GO: 0005109~frizzled binding* | 5 | 6.493506 | 6.96E−06 | 5.157576 |
| GO: 0048018~receptor agonist activity | 4 | 5.194805 | 7.12E−06 | 5.147247 |
| GO: 0000978~RNA polymerase II core promoter proximal | 9 | 11.68831 | 2.04E−05 | 4.690554 |
| region sequence-specific DNA binding | | | | |
| GO: 0003713~transcription coactivator activity | 7 | 9.090909 | 2.60E−05 | 4.584549 |
| GO: 0001078~transcriptional repressor activity, RNA | 6 | 7.792208 | 6.29E−05 | 4.201201 |
| polymerase II core promoter proximal region sequence- | | | | |
| specific binding | | | | |
| GO: 0003705~transcription factor activity, RNA | 4 | 5.194805 | 0.001251 | 2.902683 |
| polymerase II distal enhancer sequence-specific binding | | | | |
| GO: 0000979~RNA polymerase II core promoter | 4 | 5.194805 | 0.001366 | 2.864589 |
| sequence-specific DNA binding | | | | |
| GO: 0001077~transcriptional activator activity, RNA | 6 | 7.792208 | 0.001858 | 2.730969 |
| polymerase II core promoter proximal region sequence- | | | | |
| specific binding | | | | |
| GO: 0000982~transcription factor activity, RNA | 3 | 3.896104 | 0.002764 | 2.558452 |
| polymerase II core promoter proximal region sequence- | | | | |
| specific binding | | | | |
| GO: 0033613~activating transcription factor binding | 3 | 3.896104 | 0.002998 | 2.52315 |
| GO: 0046983~protein dimerization activity | 5 | 6.493506 | 0.003433 | 2.464353 |
| GO: 0003682~chromatin binding | 7 | 9.090909 | 0.004034 | 2.394239 |
| GO: 0005125~cytokine activity* | 5 | 6.493506 | 0.005233 | 2.281217 |
| GO: 0039706~co-receptor binding | 2 | 2.597403 | 0.02903 | 1.53715 |
| GO: 0005102~receptor binding | 5 | 6.493506 | 0.045458 | 1.342394 |
| GO: 0008270~zinc ion binding | 8 | 10.38961 | 0.059789 | 1.223378 |
| GO: 0071837~HMG box domain binding | 2 | 2.597403 | 0.07255 | 1.13936 |
| GO: 0000987~core promoter proximal region sequence- | 2 | 2.597403 | 0.09061 | 1.042825 |
| specific DNA binding | | | | |

| Seelcted terms | -Log10 (P value) |
|---|---|
| GO: 0043565~sequence-specific DNA binding | 15.61726584 |
| GO: 0005515~protein binding | 6.968858991 |
| GO: 0005109~frizzled binding | 5.157576351 |
| GO: 0005125~cytokine activity | 2.281217306 |

Pathway common 78 gene (KEGG_PATHWAY)

| | | | | |
|---|---|---|---|---|
| mmu04550: Signaling pathways | 8 | 10.38961 | 9.92E−08 | 7.00358251 |
| regulating pluripotency of stem cells* | | | | |
| mmu04310: Wnt signaling pathway* | 6 | 7.792208 | 4.97E−05 | 4.30322052 |
| mmu04390: Hippo signaling pathway* | 6 | 7.792208 | 6.90E−05 | 4.16089635 |
| mmu05166: HTLV-I infection | 7 | 9.090909 | 1.22E−04 | 3.91399725 |
| mmu05217: Basal cell carcinoma | 4 | 5.194805 | 5.25E−04 | 3.28011416 |
| mmu04350: TGF-beta signaling pathway* | 4 | 5.194805 | 0.001967 | 2.70625262 |
| mmu04916: Melanogenesis | 4 | 5.194805 | 0.003039 | 2.5172379 |
| mmu05205: Proteoglycans in cancer | 4 | 5.194805 | 0.021748 | 1.66258964 |

| Selected terms | ~Log10 (p value) |
|---|---|
| mmu04550: Signaling pathways regulating pluripotency of stem cells | 7.003582507 |
| mmu04310: Wnt signaling pathway | 4.303220522 |
| mmu04390: Hippo signaling pathway | 4.160896347 |
| mmu04350: TGF-beta signaling pathway | 2.706252619 |

TABLE 4
Novel dl-specific markers identified through single-cell sequencing.

| dl1 | dl2 | dl3 | dl4 | dl5 | dl6 |
|---|---|---|---|---|---|
| Lhx2 | Foxd3 | Isl1 | Pax2 | Lbx1 | Lbx1 |
| Lhx9 | Lhx1 | Prrxl1 | Lbx1 | Lmx1B | Lhx1 |
| Barhl1 | Lhx5 | Tlx3 | Lhx1 | Tlx3 | Lhx5 |
| Barhl2 | Bhlhe23 | Pkd2L1 | Lhx5 | Pou4F1 | Dmrt3 |
| Atoh1 | Angpt1 | Thsd7B | Syndig1 | C130021L20Rik | Zfp385B |
| Adgre5 | Gm12688 | Mab21L1 | Unc13C | Fibcd1 | Vsnl1 |
| Slitrk6 | Lmo3 | Rxrg | Npy | Rapgef4 | Unc13C |
| S100A6 | Hmx3 | Pou4F2 | Ptchd4 | Mab21L1 | Ptchd4 |
| Dgkk | Hmx2 | | Fxyd7 | Fgd5 | Vstm2A |
| Cd44 | Ntrk1 | | Rbm20 | Stra6L | Fxyd7 |
| Oca2 | Kcnk9 | | Pax5 | Tox2 | Rbm20 |
| Arhgap6 | Arl4D | | Pax8 | Pou4F2 | Scn9A |
| Prkg2 | Nr4A2 | | Stk32A | Tpm2 | Dgkb |
| Tiam2 | Rxrg | | Klhl14 | Skor1 | Met |
| Slc30A8 | Atf3 | | Ccdc85A | Tenm1 | Lrrc7 |
| Ptpn14 | Tenm1 | | Rxrg | Chl1 | Ccdc85A |
| Rxrg | Chac1 | | Cpm | Grp | D930028M14Rik |
| Mecom | Pnoc | | Lpcat2 | Oprl1 | Igf1 |
| Lpcat2 | Gm27199 | | Kif26A | Cnr1 | Tenm1 |
| Pcdh15 | Pou4F2 | | Slc30A3 | | Asic2 |
| Fam19A1 | Mecom | | Pnoc | | Gria1 |
| Tchh | Xylt1 | | Nebl | | Nrsn2 |
| Adamts5 | Unc5D | | Gm27199 | | Pag1 |
| Flnc | Prkar1B | | Hs3St5 | | Hs3St5 |
| P2Rx3 | Flrt3 | | Tyro3 | | Sncg |
| Col14A6 | Cnr1 | | Spats2L | | Cntn5 |
| Kctd4 | | | Gad1 | | Prkar1B |
| Rab15 | | | Fam124A | | Snca |
| Unc5D | | | Gad2 | | Chl1 |
| Grin2A | | | Cacna2D3 | | Cnr1 |
| Txlnb | | | Slc32A1 | | |
| Prkar1B | | | Skor1 | | |
| Chrna5 | | | Chl1 | | |
| Scn1A | | | Oprl1 | | |
| Sema7A | | | Cnr1 | | |
| Cbln2 | | | Cartpt | | |
| Cntn5 | | | | | |
| Cntn3 | | | | | |
| Adcy2 | | | | | |
| Ralyl | | | | | |
| Arpp21 | | | | | |
| Slc24A3 | | | | | |
| Rassf4 | | | | | |
| Cnr1 | | | | | |

TABLE 5
Mouse primer sequences for the quantitative RT-PCR analysis.

| | | SEQ | | SEQ |
|---|---|---|---|---|
| Primer | Forward Sequence | ID NO: | Reverse Sequence | ID NO: |
| GAPDH | GGCCTTCCGTGTTCCTAC | 13 | TGTCATCATACTTGGCAGGTT | 14 |
| Lhx2 | CAGCTTGCGCAAAAGACC | 15 | TAAAAGGTTGCGCCTGAACT | 16 |
| Foxd3 | CCCCAACACTGACCAACAG | 17 | GTTTGCTCCGCCAGCTTA | 18 |
| Isl1 | ATCGAGTGTTTCCGCTGTGT | 19 | ATGGGTTCGGCTGCCATTT | 20 |
| Lhx5 | CGGAGCAAAGTCTTCCACCT | 21 | GGACGACACCGAGTTGAGAC | 22 |
| Lbx1 | GCCGAAGGCCGCGATG | 23 | TGCGTGATTTTOGCCGTTTC | 24 |
| Meis1- | ACCCTGATGGACAGCCAATG | 25 | GCATACTTTGCAGCCCTGG | 26 |
| spliced | | | | |
| variant | | | | |
| A | | | | |
| Meis1- | CAATCGAAAGCCAGGGGGAA | 27 | CTCGGCTGTOCCATACTCAC | 28 |
| spliced | | | | |
| variant | | | | |
| B | | | | |
| Meis2 | GCACAGGATGCGTTTCAGTG | 29 | TGTCTAACCCATOGCCTCCA | 30 |
| Meis3 | TGGGTTCAAAGGGAAGGAAGG | 31 | GAGGTGAAGTCCTGAAGCCC | 32 |
| Irx1 | CGGCCGCGGTCACCT | 33 | CAGTTCATACTGAGAGCCCA | 34 |
| | | | TCT | |
| Irx3 | ACAGGTGACAAGAAAAATATCG | 35 | CAGCGTCCAGATGGTTCTGT | 36 |
| | C | | | |
| NeuN | ATCGTAGAGGGACGGAAAATT | 37 | GTTCCCAGGCTTCTTATTGGT | 38 |
| | GA | | C | |
| Tubb3 | ATGGACAGTGTTCGGTCTGG | 39 | AGCACCACTCTGACCAAAGA | 40 |
| | | | T | |
| Sox2 | CAAAAACCGTGATGCCGACT | 41 | CGCCCTCAGGTTTTCTCTGT | 42 |
| Pax3 | AAACCCAAGCAGGTGACAAC | 43 | GCGTCCTTGAGCAATTTGTC | 44 |
| Pax7 | TGGCCAAACTGCTGTTGATT | 45 | TAGGCTTGTCCCGTTTCCAC | 46 |
| Olig3 | ACGTTAGGGCTGTTCCAAGG | 47 | GGCAAGGGGGTAGGATGAAC | 48 |
| Wnt1 | ACTACGTTGCTACTGGCACT | 49 | GTTCACGATGCCCCACCATC | 50 |
| Wnt2 | TGATGTAGACGCAAGGGGGT | 51 | CATGTACCACCATGAAGAGO | 52 |
| | | | TGA | |
| Wnt3a | CTACCCGATCTGGTGGTCCT | 53 | ACAGAGAATGGGCTGAGTGC | 54 |
| Wnt4 | CAGGAAGGCCATCTTGACACA | 55 | GTCTTTACCTCGCAGGAGCC | 56 |
| | C | | | |
| Wnt5b | CCGGCCACCTTTCTCTCTTA | 57 | CGGAGCTCCCACGCTGAT | 58 |
| Wnt10b | ACGGTTTAAGCCCCAAGAGC | 59 | GAGTCAGTCAGCGCCTCC | 60 |
| Axin2 | GCTGCGCTTTGATAAGGTCC | 61 | CGCGAACGGCTGCTTATTTT | 62 |
| Tcf7 | GGACGGGAGCACACTTCG | 63 | GCCTTCAGGCCGTCCTCT | 64 |

### Example 2: Expansion Protocol Dorsal Neural Progenitor Cells

This Example demonstrates an exemplary expansion protocol. Embryonic stem cells are first directed to a day 4 neural precursor cell (NPC) state, and then Wnt signaling is continually switched on using CHIR, which starts the process of proliferation. The cells are then passaged every 6 days. with a 1:3 split. Control NPCs die almost immediately, while “expanded” cells keep dividing for as long as they have been passaged to date (three months). These NPCs can then be differentiated. That strategy is shown in FIG. 7J. Differentiation of the expanded cultures without any further signals yields dI3s and dI5s. A further pulse for two days with RA (day 3-5), produces dI4, dI5 and dI6. Pulsing them with RA+BMP4, yields dI1, dI2 and dW3.

Thus, a method of keeping dorsal progenitors (dPs) in a proliferative state for many passages (perhaps indefinitely) is provided. The proliferative state can be maintained without changing the fate potential of the dPs. mESCs are first patterned according to either the RA or RA+BMP4 protocol, up to day 6. After patterning, the dPs are then reintroduced to medium containing CHIR (Wnt agonist, that stimulates Wnt signaling). The CHIR-treated cultures can then serially expand 3-fold every 6 days (a passage), unlike control (DMSO-treated) cells, which start dying at passage 2 (FIG. 7). To date we have expanded dPs for 7-8 passages, i.e. a couple of months.

These expanded dPs retain patterning information such that they can be used to expand the pool of in vitro-derived dis. This is the first successful use of Wnt signaling to expand patterned dorsal neural cultures, a key step toward the long-sought goal of being able to supply specific neural populations in a limitless manner.

### Example 3: Protocol to Derive Full Diversity of Spinal Sensory Interneurons from Human PSCs

This Example demonstrates exemplary protocols to derive the full diversity of bonafide and functional spinal sensory interneurons from hPSCs through NMP intermediate progenitors. This protocol describes two directed differentiation protocols to derive full diversity of sensory spinal interneurons (dI1-dI6) from human (h) embryonic stem cells (ESCs) and induced pluripotent stem cells (iPSC) under 3-dimensional (3D) culture conditions. This protocol is based on our studies with the mouse ESCs, which we derived full diversity of mouse dIs by directing mouse ESCs through neuromesodermal intermediate (NMPs). In this protocol, we first direct hESCs/hiPSCs into NMP identity by FGF and Wnt signaling activation. Human NMPs are then converted into 3D embryoid bodies and directed towards specific dI identities using two growth factors-Retinoic acid (RA) and Bone morphogenetic protein 4(BMP4). In protocol 2.1, the addition of RA direct EBs to generate dI4-6 classes (pain, itch and heat relaying) while addition of RA+BMP4 direct EBs to generate dI1-3 (proprioceptive and mechanosensory) classes of interneurons in protocol 2.2. Both protocols generate a mixed population of bonafide dIs which are highly similar at transcriptomic level to the fetal human spinal cord interneurons, and are functional, as assessed by presence of calcium currents in day 35 old differentiated dI-containing EBs. These protocols are thus valuable tools to be used as drug testing platforms and modeling disorders affecting sensation, such as sensory processing disorders, autism and chronic pain. For modeling sensory processing disorders, any patient's iPSC lines can be used to direct patient-specific EBs containing either healthy or mutant dIs. These new classes of protocols also provide an unlimited source of bonafide human dIs for cellular replacement therapies to reestablish sensory connections in injured patients.

### Preliminary Steps

Timing: 2 days-a week:

Obtain qPCR primers (see list of Reagents and Resources below) to assess quality control and conversion efficiency. Acquire reagents.

Acquire/thaw hPSCs and maintain as undifferentiated cultures in mTeSR based medium. Cells should be passaged at least twice before starting the differentiation.

Prepare stock solutions and working aliquots. Prepare media. Note: Both ESC maintenance and differentiation media are stable up to 2 weeks at 4° C. Thus, It is not advisable to prepare all media at once, but only when needed in the specific step of the protocol.

All procedures are performed in a BSL-2 certified laboratory equipped with class II type A2 biosafety cabinets. Cultures are grown and maintained at 37° C. with 5% CO2.

### Step-by-Step Method Details

Preparing hESCs/hiPSCs Cultures for Neural Differentiation

Timing: 6-10 days

This step permits the expansion of hPSC lines for cryostorage and produces healthy pluripotent colonies of hESCs/hiPSCs suitable for neural differentiation. hESCs/hiPSCs are cultured under feeder-free conditions and passaged when cells approach 70% confluency. Cells are passaged at least twice before starting the differentiation.

Grow undifferentiated hPSCs (hESCs/hiPSCs) onto Matrigel matrix coated plates using mTesR1 media. Note that mTesR1 media needs to be changed everyday to maintain cells in undifferentiated conditions.

Passage cells 1:6 every 5 days.

Prepare one 6 well plate of undifferentiated hPSCs and grow them to reach~80% confluency for starting the differentiation. One 6-well plate is sufficient to start the differentiation. Note: (a) We found that the initial confluency of hPSCs grown on 6 well plate before starting the differentiation leads to healthy EBs (FIG. 16B, 16C). (b) Cells at 80-90% confluency should immediately be used in the differentiation procedure. Leaving cells at higher confluency for long periods (>1-2 days) can lead to spontaneous differentiation.

### Induction of Neuromesodermal (NMP) Progenitor in 2D Culture

Timing: 4-6 days

The next step is to direct pluripotent hESCs/hiPSCs towards a NMP fate, which can be assessed by the generation of SOX2+/Brachyury (T)+ NMP progenitors (Gupta et al., 2022 Cell Reports).

Prepare N2/B27 media (-Vitamin A) and pre-heat in 37° C. water bath before use.

Remove 6-well plate made in the previous step from the incubator.

Aspirate mTeSR1 medium from all wells of the 6-well plate.

Add 2 mL N2/B27 media containing 10ng of FGF2 and SuM of CHIR99021 (Wnt signaling agonist) to each well of the 6-well plate and return the plate to the incubator.

Feed cells every other day with N2/B27 media for up to 6 days.

Cells should appear as a monolayer with cells packed together tightly at the end of day 6 (FIG. 16A). This confluency is optimal for embryoid body (EB) formation in the next step.

The presence of CHIR induces a higher growth rate in the monolayer and at the end of day 6, monolayer starts showing the overgrowth seen as nodular formation throughout the wells.

Optional: After 6 days, plate can be fixed for immunohistochemistry or RNA can be isolated from the cells to check for the expression of neuromesodermal markers i.e., SOX2 and Brachyury (T).

### Embryoid Body Formation

Timing: 4 days

FIG. 16. Formation of embryoid bodies.

The next step is to grow the NMP cells as embryoid bodies (EBs) in suspended 3d culture. One 6-well differentiation plate (prepared in the previous step) can be used to seed two 24 well plates. This protocol calls for ultra-low attachment 24 well plates which prevent cells from attaching to the bottom of the plate and allow 3d embryoid body formation. Plan the experiment according to the number of 24 well plates required for all necessary end points.

Prepare enough N2B27 media with 1 μM RA. Add ROCK inhibitor only in the media for 1st day of EB formation. Note: RA stock is made in DMSO at 100 mM concentration. Dilute the stock to 1 mM in 100% ethanol. This working stock can be stored at 4° C. and can be used for up to 1 week. Note: The hESCs are vulnerable to apoptosis upon dissociation which leads to poor EB formation. Adding ROCK inhibitor Y-27632 to the cell dissociation media has been shown to significantly inhibit apoptosis and enhance the EB formation (Watanabe et al., 2007, Pettinato et al., 2015).

Pre-heat N2/B27 media in 37° C. water bath.

Remove 6-well differentiation plate from the incubator.

Add 2 ml of fresh N21B27 media containing 10 uM ROCK inhibitor in each well of the 6well plate. Incubate the cells with ROCK inhibitor for 30 minutes prior to EB formation.

Use EZ passage tool to cut the NMP monolayer into small sized pieces. The cutting process itself will make the cells detach from the plate.

Collect all cut pieces of NMP monolayer and transfer them into 50 ml conical tube.

Wait 10 minutes for the NMP pieces to settle at the bottom of the tube.

Aspirate all the remaining media, leaving a small amount at the bottom so that the pellet of NMP pieces is not disturbed.

Add 50 ml of fresh N2/B27 containing 1 uM RA in the tube and resuspend the NMP pieces uniformly.

Transfer 1 mL of N2/B27 media+NMP pieces into each well of the ultra-low attachment 24 well plate to promote EB formation.

Return 24 well-plates to 37° C. After 2 days, the resuspended cells should have formed small EBs in each well (FIG. 16B, 16C).

Change the medium on EB cultures on the next day and then every 2 days.

### Induction of Dorsal Sensory Interneurons

Timing: 7 days.

This step directs 3d EBs towards different cell fates, i.e. dI4-6 (protocol 2.1) or dI1-3 (protocol 2.2), by the addition of either RA alone or both RA and BMP4 (RA+BMP4).

Prepare N2/B27 media supplemented with either 1 μM RA for protocol 1 or 1 μM RA+10 ng/mL human recombinant BMP4 for protocol 2.

Pre-heat medium in 37° C. water bath.

Protocol 2.1: Induce dI4, dI5, and dI6 by transferring EBs into N2/B27 media+1 μM RA on day 10.

Protocol 2.2: Induce dI1, dI2, and dI3s by transferring EBs into N21B27 media with 1 μM RA+10 ng/mL human recombinant BMP4 between day 8-day 10.

Resuspend EBs and transfer 1 mL EBs+medium to low attachment 24-well plate.

Transfer EBs to fresh N2/B27 media by collecting EBs in 50 mL conical tube.

Allow EBs to settle at the bottom of the tube by gravity. This step will take 5-10 mins.

Aspirate the liquid from the tube and resuspend in either N2/B27 media+1 μM RA (Protocol 2.1) or N2/B27 media with 1 μM RA+10 ng/mL BMP4 (Protocol 2.2).

Feed EBs by transferring them into fresh N2/B27 media+growth factors after every 2 days.

### Neuronal Differentiation and Maturation

Timing: 19 days.

This step permits dorsal EBs to differentiate and then mature into dI4-6 and dI1-3 neurons. EBs are progressed through both differentiation and maturation media.

Differentiation media contain a notch inhibitor DAPT which enhances neuronal differentiation along with RA (protocol 2.1) and RA+BMP4 (protocol 2.2). The maturation media contain Ascorbic acid to enhance the differentiation process and growth factors such as 10 ng/ml BDNF, ng/ml NGF, and 10 ng/ml CNTF to maintain high survival rates of dIs.

At the end of the protocol, EBs can either be fixed, sectioned, and stained directly, or dissociated and plated onto laminin coated slides to examine the neuronal morphology. To monitor the functionality of dIs in EBs, EBs can be infected with adenovirus containing calcium sensors (AAV1: synapsin: gCaMP6 f). This specific adenovirus encodes calcium sensor gCaMP6F under the neuronal promoter synapsin, so the expression of gCaMP6F is only directed in the differentiated neurons.

Prepare neural differentiation medium (NBDM) containing 10 uM DAPT supplemented with either 1 μM RA for protocol 2.1 or 1 μM RA+10 ng/mL human recombinant BMP4 for protocol 2.2.

Transfer EBs from protocol 2.1 into NBDM supplemented with 1 μM RA. Transfer EBs from protocol 2.2 into NBDM supplemented with 1 μM RA+10 ng/mL BMP4.

Transfer EBs to fresh NBDM medium supplemented with growth factors after every 2 days. On day 28, transfer EBs from protocol 2.1 and protocol 2.2 into maturation medium supplemented with Ascorbic acid, BDNF, GDNF, CNTF and NGF. Repeat, now with maturation medium, to continue feeding EBs every 2 days until day 36 or any earlier desired endpoint for the quality assessment and functional analysis.

At the end of the differentiation, EBs can either be a) fixed directly or dissociated for immunostaining, see steps 39-65 or b) lysed to obtain RNA for a qPCR analysis. EBs can be maintained in the maturation media for longer than day 36 timepoint. We have successfully grown and maintained EBs in maturation media up to day 50, but longer timepoints should also be possible. Optional: EBs can be dissociated at the end of the differentiation using 0.25% Trypsin or papain solution to obtain single-cell suspension and plated onto laminin coated coverslips or IbidiTM chamber slides for immunostaining procedures.

### Viral Tagging of EBs for Functional Assays and Calcium Imaging

Collect 30-60-day old mature EBs from either protocol 2.1 or protocol 2.2 for viral tagging. Take at least 5-6 EBs from each protocol and transfer them into a new well of 24 well plate. At this point, the low attachment plate is optional, because mature EBs do not attach to the plastic wells of 24 well plates.

Thaw a 5 μl aliquot of concentrated AAV1::Synapsin::gCaMP6F virus of 1013 titer on ice.

Add 250 μl of maturation media in the virus containing vial and mix the media thoroughly by pipetting to distribute virus thoroughly.

Remove the remaining media from the wells containing EBs.

Add virus+maturation media directly into the wells containing EBs.

Incubate EBs overnight with the virus+media solution.

Next day, add additional 750 μl maturation media in the wells of viral transduced EB.

Replenish with the fresh 1 ml of maturation media every other day for 2 weeks post virus transduction.

At the end of two weeks, the GFP should be visible in the EBs, indicating the successful virus infection in the neurons.

Once GFP is visible, EBs are ready to be used for imaging on 2 photon microscope to record calcium currents in the neurons.

For calcium imaging, EBs are mounted on the glass bottom dish in Matrigel matrix, bathed in artificial cerebrospinal fluid containing 100 uM kainic acid to induce spontaneous activity of neurons.

### Quality Control Analyses of EBs

Immunostaining analysis of fixed EBs:

Prepare fresh 4% PFA in 1× PBS to fix the EBs.

Collect EBs in a 50 mL conical tube, allow them to settle at the bottom of the tube. Remove medium and wash EBs with 1× PBS. Allow EBs to settle at the bottom of the tube by gravity and aspirate PBS from the tube.

Add 5-10 mL 4% PFA in the tube and incubate EBs on an orbital shaker at room temperature for 20 minutes.

Wash EBs twice with 1× PBS.

Embedding for cryo-sectioning: replace PBS with 30% sucrose and allow EBs to equilibrate and settle at the bottom of the tube. This step can take from 30 minutes to 1 hr.

Carefully transfer the EBs using wide bore pipette tips into a cryo-block filled with OCT (optimum cutting compound). Gently swirl them around in the OCT using 19-20G hypodermal needle to equilibrate and arrange EBs at bottom-center of the block. Transfer blocks onto dry ice to freeze. Note: Aspirating all EBs in a 100 μl pipette volume will minimize the amount of sucrose transferred into the OCT.

Prepare 12 μm sections of EBs on glass slides, using standard cryo-sectioning methods. Incubate slides with an antibody blocking solution (10% heat inactivated serum in 1× PBS+0.2% triton) for 30 minutes.

Dilute desired primary antibodies in the antibody blocking solution. Replace blocking solution, with 500 μl of diluted antibody solution/slide and incubate overnight at 4° C. in a humidified chamber. Remove primary antibody and wash slides twice with 1×PBS+0.2% triton (PBTN).

Add species appropriate secondary antibodies in PBTN and incubate slides for 1 hr in secondary antibody. Remove secondary antibody solution and wash slides twice with PBTN.

Counterstain with DAPI to detect nuclei. Coverslip slides using either Vectorshield™ or ProLong™ Gold mounting media. Examine slides using a microscope. Cell numbers can be counted using the ImageJ cell counter plugin.

qPCR Analysis:

Collect RNA lysate for RNA extraction. Note: this method of RNA isolation has been optimized for the Qiagen RNA extraction kit. Other RNA extraction methods can be used.

Those skilled in the art understand that the protocol may need modification, and can make adjustments accordingly.

Collect EBs in a 15 mL conical tube. Wash once with 1× PBS. Remove PBS and add 500 ul RLT buffer (Qiagen).

Dissociate EBs in RLT buffer by passing through QIAshredder (Qiagen). Collect the elutant and proceed for RNA isolation according to the manufacturer's instructions.

Prepare cDNA samples from the isolated RNA according to the manufacturer's instructions.

Set up 10 μl qRT-PCR reaction with SYBR green using the following cycling parameters:

qRT-PCR setup reaction (10 μl)

| Steps | Temperature | Time | Cycles | |
|---|---|---|---|---|
| Initial denaturation | 92° C. | 2 mins | 1 | |
| Denaturation | 92° C. | 15 sec | | |
| | | | {close oversize brace} | 40 cycles |
| Annealing | 60° C. | 1 min | | |

| | | |
|---|---|---|
| Extension | 72° C. | 1 min |
| Hold | 4° C. | Forever |

Analyze qPCR results using the ΔΔ CT method (Livak and Schmittgen, 2001).

### Expected Outcomes

FIG. 17: Immunostaining of D36 embryoid bodies to detect dI4-6, dI1 and dI3 neurons.

The anticipated outcome of this protocol is as follows. Protocol 1 should yield 30-40% of the population of LHX1/5+PAX2+/TUJ1+dI4-d6 classes of neurons and 30-40% LMXB+d5. Protocol 2 should yield 20-30% of LHX2+dI1s, 20% FOXD3 dI2, and 10% IsI1+TLX3+TUJ1+d3s neurons. In addition to the cell fate specific transcription factors expressed by these dI populations, they should also express pan-neural marker-beta III tubulin (TUJ1) and spinal cord specific axonal markers such Dcc and Robo3 (see (Gupta et al., 2018) for more information and images of the axonal markers).

### Reagents and Resources

| | | |
|---|---|---|
| REAGENT or RESOURCE | SOURCE | IDENTIFIER |

Antibodies

| | | |
|---|---|---|
| Goat polyclonal anti-Sox2 | Santa Cruz Biotech. | SC-17320 |
| Rabbit polyclonal anti-Nanog | Cell Signaling | D73G4 |
| Mouse monoclonal anti-Pax6 | DSHB | AB528427 |
| Goat polyclonal anti-Pax3 | R&D Systems | AF2457 |
| Goat polyclonal anti-HoxA5 | Santa Cruz Biotech. | SC-13199 |
| Mouse monoclonal anti-Tubb3 | BioLegend | 801202 |
| Goat polyclonal anti-Lhx2 | Santa Cruz Biotech. | Sc-19344 |
| Goat polyclonal anti-Isl1 | R&D Systems | AF1837 |
| Mouse monoclonal anti-Lhx1/5 | DSHB | AB531784 |
| Rabbit polyclonal anti-Pax2 | Invitrogen | PA5-81235 |
| Goat polyclonal anti-Robo3 | R&D Systems | AF3076 |
| Goat polyclonal anti-DCC | R&D Systems | AF844 |

Chemicals, Peptides, and Recombinant Proteins

| | | |
|---|---|---|
| mTeSR1 complete kit (basal media | Stem Cell | 85850 |
| plus 5x supplement) | | |
| | Technologies | |
| Dulbecco's Modified Eagle Medium/ | Hyclone | SH30023.02 |
| Nutrient Mixture F-12 (DMEM-F12) | | |
| Neurobasal Media | Thermo Fisher | 21103049 |
| Iscove's Modified Dulbecco's Medium (IMDM) | Thermo Fisher | 12440053 |
| Non-essential amino acid (NEAA) (100x) | Thermo Fisher | 11140050 |
| Penicillin/Streptomycin (Pen-Strep) | Thermo Fisher | 15140122 |
| Glutamax (100x) | Thermo Fisher | 35050 |
| N2 supplement (100x) | Thermo Fisher | 17502-048 |
| B27 supplement (50x) | Thermo Fisher | 17504-044 |
| B27 supplement without vitamin A (100x) | Thermo Fisher | 12587010 |
| Accutase (100 mL) | Stem Cell | 7920 |
| | Technologies | |
| ReLeSR | Stem Cell | 05872 |
| | Technologies | |
| Ascorbic acid | Sigma-Aldrich | A8960-5G |
| Geltrex ™ | Thermo Fisher | A1413301 |
| Human recombinant bone morphogenetic protein 4 | Thermo Fisher | PHC9534 |
| (BMP4) | | |
| Retinoic acid | Sigma-Aldrich | R2625 |
| Rho kinase (ROCK) inhibitor (Y-27632), 10 mM | BioPioneer | SM-008 |
| Primocin | Invitrogen | Ant-pm-05 |
| Dulbecco (d) PBS (without Calcium, magnesium) | Thermo Fisher | 14190250 |
| Poly-L-ornithine | Sigma-Aldrich | P4957 |
| Laminin | Sigma-Aldrich | L2020 |
| Dorsomorohin | Sigma-Aldrich | P5499 |
| SB431542 | Stemgent | 04-0010-10 |

Experimental Models: Cell Lines

| | |
|---|---|
| H9 hESCs | WA09, NIH registry |
| | #0062 |
| IPSC lines | |

Oligonucleotides

| | |
|---|---|
| HOXA5 F-AAGTCATGACAACATAGGCGG | SEQ ID NO: 1 |
| R-TTCAATCCTCCTTCTGCGGG | SEQ ID NO: 2 |
| LHX2 F-TGGACCGAGGAACAACTTGG | SEQ ID NO: 3 |
| R-TCGCTCAGTCCACAAAACTG | SEQ ID NO: 4 |
| ISL1 F-GATTTGGAATGGCATGCGGC | SEQ ID NO: 5 |
| R-GCGCATTTGATCCCGTACAA | SEQ ID NO: 6 |
| LHX1 F-CAACATGCGCGTCATTCAGG | SEQ ID NO: 7 |
| R-ACTCGCTCTGGTAATCTCOG | SEQ ID NO: 8 |
| LHX5 F-GCGTCATCCAGGTGTGGTTT | SEQ ID NO: 9 |
| R-GGTGGACCCCAACATCTCAG | SEQ ID NO: 10 |
| TUBB3 F-GGCCAAGGGTCACTACACG | SEQ ID NO: 11 |
| R-GCAGTCGCAGTTTTCACACTC | SEQ ID NO: 12 |

Other

| | | |
|---|---|---|
| 6-well plate | Coming | 490007-412 |
| Ultra-low-attachment 24-well plate | Coming | CLS3473 |
| Optimum cutting temperature (OCT) compound | Sakura-Finetek | 4583 |
| Peel-A-Way Embedding molds | Polysciences Inc. | 18986-1 |
| EZ Passage tool | Thermo Fisher | 23181010 |

Growth factor/Cytokine stock solutions

Retinoic acid (RA) stock solution (100 mM)

| Reagent | Final Concentration (mM or μM) | Amount |
|---|---|---|
| All trans RA | | 50 mg |
| DMSO | | 1.66 mL |
| Total | 100 mM in DMSO | 1.66 mL |

Retinoic acid (RA) is a light sensitive compound. Work under low-light conditions when working with RA. To make a less concentrated solution, dilute RA stock in 100% ethanol. Retinoic acid (RA) is a potent teratogen. Dispose concentrated stock solutions responsibly according to the manufacturer's recommendations.

BMP4 stock solution (10 μg/) mL

| Reagent | Final Concentration (mM or μM) | Amount |
|---|---|---|
| BMP4 | 10 μg/mL | 10 μg |
| 4 mM HCl with 0.1% BSA | | 1 mL |
| Total | | 1 mL |

Make 25 μl aliquots and avoid multiple freeze-thaw cycles. Aliquots can be stored at −30° C. for up to 1 year.

Ascorbic acid stock solution (10 mg/) mL:

| Reagent | Final Concentration (mM or μM) | Amount |
|---|---|---|
| Ascorbic acid | 10 mg/mL | 10 mg |
| dPBS | | 1 mL |
| Total | | 1 mL |

Ascorbic acid is light sensitive. Protect the stock solution from light. Make 20 μL working aliquots to prevent frequent freeze-thaw. Aliquots can be stored at −20° C. for up to 6 months.

Dibutyryl cyclic-AMP stock solution (10 mM):

| Reagent | Final Concentration (mM or μM) | Amount |
|---|---|---|
| Dibutyryl cyclic-AMP salt | 10 mM | 10 mg |
| ddH2O | n/a | 2 mL |
| Total | n/a | 2 mL |

Determine solute required to make 10 mM solution from the manufacturer's datasheet as batch-to-batch variation may affect the volume of water required to dissolve cAMP. Store aliquots at −20° C.

### Maintenance and Differentiation Media Composition

mTeSR1 medium (commercially available):

| Reagent | Final Concentration (mM or μM) | Amount |
|---|---|---|
| Basal media | — | 400 mL |
| 5× supplement | 1× | 100 mL |
| Total | | 500 mL |

Store complete media at 2-8° C. for up to 2 weeks or −20° C. for up to 6 months. Avoid warming the media for the extended period of time in the 37° C. water bath due to bFGF instability.

N2/B27 media:

| Reagent | | Amount |
|---|---|---|
| IMDM | — | 470 mL |
| N2 supplement | 1% (v/v) | 5 mL |
| B27 supplement (-Vit A) | 2% (v/v) | 10 mL |
| Primocin | 1× | 1 mL |
| Glutamax (100×) | 1× | 5 mL |
| NEAA (100×) | 1× | 5 mL |
| Total | | 500 mL |

Store complete media at 2-8° C. for up to 2 weeks.

NBD medium (NBDM):

| | Final Concentration | |
|---|---|---|
| Reagent | (mM or μM) | Amount |
| Neurobasal media | — | 470 mL |
| N2 supplement | 1% (v/v) | 5 mL |
| B27 supplement with Vit A | 2% (v/v) | 10 mL |
| Primocin | 1× | 1 mL |
| Non-essential amino acid (NEAA) | 1× | 5 mL |
| Glutamax | 1× | 5 mL |
| Total | | 500 mL |

Store complete media at 2-8° C. for up to 2 weeks. Note: The B27 supplement is commercially available with and without Vitamin A (retinyl acetate). This media includes Vitamin A containing B27 supplement because Vitamin A is known to enhance the neuronal differentiation and maturation in the stem cell cultures.

Maturation medium

| | Final Concentration | |
|---|---|---|
| Reagent | (mM or μM) | Amount |
| DMEM: F12 | — | 475 mL |
| N2 supplement | 1% | 5 mL |
| B27 supplement with Vit A | 2% | 10 mL |
| Pen-Strep | 1× | 5 mL |
| Non-essential amino acid (NEAA) | 1× | 5 mL |
| Dibutyric cyclic AMP stock solution | 1 μM | 50 uL |
| Ascorbic acid stock solution | 200 ng/ml | 10 uL |
| Total | | 500 mL |

Store complete media at 2-8° C. for up to 2 weeks.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. A method for producing dorsal spinal interneurons (dI) in vitro, the method comprising:
(a) contacting a population of neuromesodermal progenitor cells (NMPs) with retinoic acid (RA) for a sufficient time to neutralize the NMPs into dorsal neural progenitor cells (NPCs); and
(b) culturing the dorsal NPCs of (a) with retinoic acid (RA) for at least one additional day, and optionally, further contacting the dorsal NPCs with bone morphogenic protein 4 (BMP4) for the at least one additional day,
wherein the contacting of step (a) is performed in the absence of BMP4.

2. A method for producing dorsal spinal interneurons (dI) in vitro, the method comprising:
(a) contacting a population of dorsal neural progenitor cells (NPCs) with an activator of the Wnt/beta-catenin pathway; and
(b) culturing the dorsal NPCs of (a) under conditions permitting proliferation,
wherein the dorsal NPCs maintain their dI fate potential.

3. The method of claim 2, wherein the activator of the Wnt/beta-catenin pathway is CHIR (C₂₂H₁₈Cl₂N₈; CAS No. 252917-06-9).

4. The method of claim 2, wherein the dorsal NPCs are obtained by contacting a population of NMPs with retinoic acid (RA) for about two days, and optionally, with bone morphogenic protein 4 (MP4) for the second of the two days.

5. The method of claim 2, further comprising:
(c) subsequently ceasing the contacting of step (a) followed by:
(d) contacting the dorsal NPCs cultured in (b) with retinoic acid (RA) for about two days, and optionally, with bone morphogenic protein 4 (BMP4) for the second of the two days.

6. The method of claim 1, wherein the dorsal NPCs comprise a plurality of each of dI1, dI2, dI3, dI4, dI5, and dI6 phenotypes.

7. The method of claim 1, wherein the dis produced by the method comprise at least 5% dI1, dI2, dI3, dI4, dI5, or dI6.

8. The method of claim 1, wherein the dis produced by the method comprise dI2 and/or dI5.

9. The method of claim 1, wherein the dorsal NPCs contacted with RA express Pax3 and Pax7, and wherein the dorsal NPCs contacted with RA and BMP4 express Pax3 and Olig3.

10. The method of claim 1, wherein the dorsal NPCs express one or more dI markers listed in Table 4.

11. The method of claim 1, wherein the NMPs are obtained by contacting a population of embryonic stem cells (ESCs) with bFGF and CHIR for about 3 days.

12. A method of screening neuroactive agents, the method comprising:
(a) contacting a population of dorsal spinal interneurons (dIs) produced by the method of claim 1 with a candidate neuroactive agent; and
(b) measuring a change in physicochemical properties of the dIs contacted in step (a) relative to a reference population of dis,
wherein a neuroactive agent is identified when a change relative to the reference population of dIs is measured in step (b).

13. The method of claim 12, wherein the candidate neuroactive agent is an analgesic, and the population of dIs is enriched for dI4 and/or dI5.

14. The method of claim 12, wherein the population of dIs has been enriched for dI4 and dI5 by contacting the dorsal NPCs with retinoic acid (RA) for about two days, and subsequently culturing the dorsal NPCs in the absence of a growth factor.

15. The method of claim 12, wherein the candidate neuroactive agent is a psychoactive compound.

16. The method of claim 1, wherein the dI are murine or human dI.

17. A method of transplanting dorsal NPCs to the spinal cord of a subject in need thereof, the method comprising administering dorsal NPCs produced by the method of claim 1 to the subject.

18. The method of claim 17, wherein the dorsal NPCs are administered by injection to the spinal cord of the subject.

19. The method of claim 1, wherein the culturing is three-dimensional culturing.

20. An ex vivo model for disorders affecting sensation, the model comprising producing dorsal spinal interneurons (dI) according to the method of claim 19 under conditions sufficient for formation of embryoid bodies (EBs).

21. The ex vivo model of claim 20, wherein the NMPs are obtained from a population of induced pluripotent stem cells (iPSCs).
